SEQUENCE LISTING (1065 sequences)

SEQ ID NO: 1 - protein, 6 aa, Artificial Sequence
  Synthetic peptide
SVNVDL

SEQ ID NO: 2 - protein, 6 aa, Artificial Sequence
  Synthetic peptide
SLNVDV

SEQ ID NO: 3 - protein, 6 aa, Artificial Sequence
  Synthetic peptide
SVDVNL

SEQ ID NO: 4 - protein, 6 aa, Artificial Sequence
  Synthetic peptide
DLSVVL

SEQ ID NO: 5 - protein, 6 aa, Artificial Sequence
  Synthetic peptide
SVNLDV

SEQ ID NO: 6 - protein, 6 aa, Artificial Sequence
  Synthetic peptide
SVVNDV

SEQ ID NO: 7 - protein, 6 aa, Artificial Sequence
  Synthetic peptide
DVSLVN

SEQ ID NO: 8 - protein, 6 aa, Artificial Sequence
  Synthetic peptide
DVSVLN

SEQ ID NO: 9 - protein, 6 aa, Artificial Sequence
  Synthetic peptide
SDLVNV

SEQ ID NO: 10 - protein, 6 aa, Artificial Sequence
  Synthetic peptide
SLNVVS

SEQ ID NO: 11 - protein, 6 aa, Artificial Sequence
  Synthetic peptide
LNVDSV

SEQ ID NO: 12 - protein, 6 aa, Artificial Sequence
  Synthetic peptide
NDLSVV

SEQ ID NO: 13 - protein, 6 aa, Artificial Sequence
  Synthetic peptide
VDNLVS

SEQ ID NO: 14 - protein, 6 aa, Artificial Sequence
  Synthetic peptide
VNDVSL

SEQ ID NO: 15 - protein, 6 aa, Artificial Sequence
  Synthetic peptide
VSDNVL

SEQ ID NO: 16 - protein, 6 aa, Artificial Sequence
  Synthetic peptide
LNDVVS

SEQ ID NO: 17 - protein, 6 aa, Artificial Sequence
  Synthetic peptide
LSVDVN

SEQ ID NO: 18 - protein, 6 aa, Artificial Sequence
  Synthetic peptide
NSVDLV

SEQ ID NO: 19 - protein, 6 aa, Artificial Sequence
  Synthetic peptide
VDVLNS

SEQ ID NO: 20 - protein, 6 aa, Artificial Sequence
  Synthetic peptide
VNDSVL

SEQ ID NO: 21 - protein, 6 aa, Artificial Sequence
  Synthetic peptide
VNVSLD

SEQ ID NO: 22 - protein, 6 aa, Artificial Sequence
  Synthetic peptide
LDVNSV

SEQ ID NO: 23 - protein, 6 aa, Artificial Sequence
  Synthetic peptide
LNVVDS

SEQ ID NO: 24 - protein, 6 aa, Artificial Sequence
  Synthetic peptide
LSDVVN

SEQ ID NO: 25 - protein, 6 aa, Artificial Sequence
  Synthetic peptide
LVVNDS

SEQ ID NO: 26 - protein, 6 aa, Artificial Sequence
  Synthetic peptide
NSLDVV

SEQ ID NO: 27 - protein, 6 aa, Artificial Sequence
  Synthetic peptide
VLVDNS

SEQ ID NO: 28 - protein, 6 aa, Artificial Sequence
  Synthetic peptide
VNLSDV

SEQ ID NO: 29 - protein, 6 aa, Artificial Sequence
  Synthetic peptide
VNVDLS

SEQ ID NO: 30 - protein, 6 aa, Artificial Sequence
  Synthetic peptide
DNVSVD

SEQ ID NO: 31 - protein, 6 aa, Artificial Sequence
  Synthetic peptide
LSDNVV

SEQ ID NO: 32 - protein, 6 aa, Artificial Sequence
  Synthetic peptide
LSVVDN

SEQ ID NO: 33 - protein, 6 aa, Artificial Sequence
  Synthetic peptide
VLDVSN

SEQ ID NO: 34 - protein, 6 aa, Artificial Sequence
  Synthetic peptide
VNDLVS

SEQ ID NO: 35 - protein, 6 aa, Artificial Sequence
  Synthetic peptide
VNVSDL

SEQ ID NO: 36 - protein, 6 aa, Artificial Sequence
  Synthetic peptide
VVLSDN

SEQ ID NO: 37 - protein, 6 aa, Artificial Sequence
  Synthetic peptide
LVSVNL

SEQ ID NO: 38 - protein, 6 aa, Artificial Sequence
  Synthetic peptide
LVNVSV

SEQ ID NO: 39 - protein, 6 aa, Artificial Sequence
  Synthetic peptide
SLNVSV

SEQ ID NO: 40 - protein, 6 aa, Artificial Sequence
  Synthetic peptide
LVSVNS

SEQ ID NO: 41 - protein, 6 aa, Artificial Sequence
  Synthetic peptide
SVDVNV

SEQ ID NO: 42 - protein, 6 aa, Artificial Sequence
  Synthetic peptide
LVVSVL

SEQ ID NO: 43 - protein, 6 aa, Artificial Sequence
  Synthetic peptide
VNLVVS

SEQ ID NO: 44 - protein, 6 aa, Artificial Sequence
  Synthetic peptide
SNLVSV

SEQ ID NO: 45 - protein, 6 aa, Artificial Sequence
  Synthetic peptide
SVNVLS

SEQ ID NO: 46 - protein, 6 aa, Artificial Sequence
  Synthetic peptide
VLVSVL

SEQ ID NO: 47 - protein, 6 aa, Artificial Sequence
  Synthetic peptide
LVNVSL

SEQ ID NO: 48 - protein, 6 aa, Artificial Sequence
  Synthetic peptide
SVNVDS

SEQ ID NO: 49 - protein, 6 aa, Artificial Sequence
  Synthetic peptide
VLSVNV

SEQ ID NO: 50 - protein, 6 aa, Artificial Sequence
  Synthetic peptide
NLVVSV

SEQ ID NO: 51 - protein, 6 aa, Artificial Sequence
  Synthetic peptide
SVVLNV

SEQ ID NO: 52 - protein, 6 aa, Artificial Sequence
  Synthetic peptide
LSVVNL

SEQ ID NO: 53 - protein, 6 aa, Artificial Sequence
  Synthetic peptide
SNLVVS

SEQ ID NO: 54 - protein, 6 aa, Artificial Sequence
  Synthetic peptide
SVVLDV

SEQ ID NO: 55 - protein, 6 aa, Artificial Sequence
  Synthetic peptide
LVSLNV

SEQ ID NO: 56 - protein, 6 aa, Artificial Sequence
  Synthetic peptide
VSLNVV

SEQ ID NO: 57 - protein, 6 aa, Artificial Sequence
  Synthetic peptide
VKVQIY

SEQ ID NO: 58 - protein, 6 aa, Artificial Sequence
  Synthetic peptide
QVVIYK

SEQ ID NO: 59 - protein, 6 aa, Artificial Sequence
  Synthetic peptide
KVIQVY

SEQ ID NO: 60 - protein, 6 aa, Artificial Sequence
  Synthetic peptide
VYVKIY

SEQ ID NO: 61 - protein, 6 aa, Artificial Sequence
  Synthetic peptide
QIVVYK

SEQ ID NO: 62 - protein, 6 aa, Artificial Sequence
  Synthetic peptide
QVIKVY

SEQ ID NO: 63 - protein, 6 aa, Artificial Sequence
  Synthetic peptide
VQVKIY

SEQ ID NO: 64 - protein, 6 aa, Artificial Sequence
  Synthetic peptide
QVVKIY

SEQ ID NO: 65 - protein, 6 aa, Artificial Sequence
  Synthetic peptide
QVIVYK

SEQ ID NO: 66 - protein, 6 aa, Artificial Sequence
  Synthetic peptide
QIVKVY

SEQ ID NO: 67 - protein, 6 aa, Artificial Sequence
  Synthetic peptide
QKIVVY

SEQ ID NO: 68 - protein, 6 aa, Artificial Sequence
  Synthetic peptide
QKVVYI

SEQ ID NO: 69 - protein, 6 aa, Artificial Sequence
  Synthetic peptide
KVQVYI

SEQ ID NO: 70 - protein, 6 aa, Artificial Sequence
  Synthetic peptide
QVVKYI

SEQ ID NO: 71 - protein, 6 aa, Artificial Sequence
  Synthetic peptide
KVQIYV

SEQ ID NO: 72 - protein, 6 aa, Artificial Sequence
  Synthetic peptide
VKIQVY

SEQ ID NO: 73 - protein, 6 aa, Artificial Sequence
  Synthetic peptide
VIQKVY

SEQ ID NO: 74 - protein, 6 aa, Artificial Sequence
  Synthetic peptide
KVVIYK

SEQ ID NO: 75 - protein, 6 aa, Artificial Sequence
  Synthetic peptide
QIVKYV

SEQ ID NO: 76 - protein, 6 aa, Artificial Sequence
  Synthetic peptide
QKVIYV

SEQ ID NO: 77 - protein, 6 aa, Artificial Sequence
  Synthetic peptide
KQVVIY

SEQ ID NO: 78 - protein, 6 aa, Artificial Sequence
  Synthetic peptide
KIQVYV

SEQ ID NO: 79 - protein, 6 aa, Artificial Sequence
  Synthetic peptide
KVYVQI

SEQ ID NO: 80 - protein, 6 aa, Artificial Sequence
  Synthetic peptide
VVQIYK

SEQ ID NO: 81 - protein, 6 aa, Artificial Sequence
  Synthetic peptide
KQVIVY

SEQ ID NO: 82 - protein, 6 aa, Artificial Sequence
  Synthetic peptide
VQIKVY

SEQ ID NO: 83 - protein, 6 aa, Artificial Sequence
  Synthetic peptide
QKIVYV

SEQ ID NO: 84 - protein, 6 aa, Artificial Sequence
  Synthetic peptide
VIQVYK

SEQ ID NO: 85 - protein, 6 aa, Artificial Sequence
  Synthetic peptide
KVVIQY

SEQ ID NO: 86 - protein, 6 aa, Artificial Sequence
  Synthetic peptide
KVQVIY

SEQ ID NO: 87 - protein, 6 aa, Artificial Sequence
  Synthetic peptide
QYVVIK

SEQ ID NO: 88 - protein, 6 aa, Artificial Sequence
  Synthetic peptide
YVVIQK

SEQ ID NO: 89 - protein, 6 aa, Artificial Sequence
  Synthetic peptide
KIVVQY

SEQ ID NO: 90 - protein, 6 aa, Artificial Sequence
  Synthetic peptide
YQVIVK

SEQ ID NO: 91 - protein, 6 aa, Artificial Sequence
  Synthetic peptide
KIYVQV

SEQ ID NO: 92 - protein, 6 aa, Artificial Sequence
  Synthetic peptide
KVVIYQ

SEQ ID NO: 93 - protein, 6 aa, Artificial Sequence
  Synthetic peptide
KYVVQI

SEQ ID NO: 94 - protein, 6 aa, Artificial Sequence
  Synthetic peptide
YQIVVK

SEQ ID NO: 95 - protein, 6 aa, Artificial Sequence
  Synthetic peptide
YVVIQY

SEQ ID NO: 96 - protein, 6 aa, Artificial Sequence
  Synthetic peptide
KQVIYV

SEQ ID NO: 97 - protein, 6 aa, Artificial Sequence
  Synthetic peptide
KYVQVI

SEQ ID NO: 98 - protein, 6 aa, Artificial Sequence
  Synthetic peptide
KYVVIQ

SEQ ID NO: 99 - protein, 6 aa, Artificial Sequence
  Synthetic peptide
QKVVIY

SEQ ID NO: 100 - protein, 6 aa, Artificial Sequence
  Synthetic peptide
QYVIVK

SEQ ID NO: 101 - protein, 6 aa, Artificial Sequence
  Synthetic peptide
IVQKVY

SEQ ID NO: 102 - protein, 6 aa, Artificial Sequence
  Synthetic peptide
KYIVVQ

SEQ ID NO: 103 - protein, 6 aa, Artificial Sequence
  Synthetic peptide
QVIKYV

SEQ ID NO: 104 - protein, 6 aa, Artificial Sequence
  Synthetic peptide
YIVVQK

SEQ ID NO: 105 - protein, 6 aa, Artificial Sequence
  Synthetic peptide
YQVVIK

SEQ ID NO: 106 - protein, 6 aa, Artificial Sequence
  Synthetic peptide
IKVQVY

SEQ ID NO: 107 - protein, 6 aa, Artificial Sequence
  Synthetic peptide
KVVQYI

SEQ ID NO: 108 - protein, 6 aa, Artificial Sequence
  Synthetic peptide
KYVQIV

SEQ ID NO: 109 - protein, 6 aa, Artificial Sequence
  Synthetic peptide
VKQVYI

SEQ ID NO: 110 - protein, 6 aa, Artificial Sequence
  Synthetic peptide
VQVIYK

SEQ ID NO: 111 - protein, 6 aa, Artificial Sequence
  Synthetic peptide
VVIQKY

SEQ ID NO: 112 - protein, 6 aa, Artificial Sequence
  Synthetic peptide
IQVVYK

SEQ ID NO: 113 - protein, 6 aa, Artificial Sequence
  Synthetic peptide
KIVVYQ

SEQ ID NO: 114 - protein, 6 aa, Artificial Sequence
  Synthetic peptide
KYQVIV

SEQ ID NO: 115 - protein, 6 aa, Artificial Sequence
  Synthetic peptide
KYVIVQ

SEQ ID NO: 116 - protein, 6 aa, Artificial Sequence
  Synthetic peptide
VVYIQK

SEQ ID NO: 117 - protein, 6 aa, Artificial Sequence
  Synthetic peptide
IVQVYK

SEQ ID NO: 118 - protein, 6 aa, Artificial Sequence
  Synthetic peptide
KQIVYV

SEQ ID NO: 119 - protein, 6 aa, Artificial Sequence
  Synthetic peptide
KYVIQV

SEQ ID NO: 120 - protein, 6 aa, Artificial Sequence
  Synthetic peptide
QKVIVY

SEQ ID NO: 121 - protein, 6 aa, Artificial Sequence
  Synthetic peptide
QYIKVV

SEQ ID NO: 122 - protein, 6 aa, Artificial Sequence
  Synthetic peptide
QYIVKV

SEQ ID NO: 123 - protein, 6 aa, Artificial Sequence
  Synthetic peptide
VQIVYK

SEQ ID NO: 124 - protein, 6 aa, Artificial Sequence
  Synthetic peptide
VVQKYI

SEQ ID NO: 125 - protein, 6 aa, Artificial Sequence
  Synthetic peptide
VYQIVK

SEQ ID NO: 126 - protein, 6 aa, Artificial Sequence
  Synthetic peptide
YVQVIK

SEQ ID NO: 127 - protein, 6 aa, Artificial Sequence
  Synthetic peptide
QIVYVK

SEQ ID NO: 128 - protein, 6 aa, Artificial Sequence
  Synthetic peptide
IKVYQV

SEQ ID NO: 129 - protein, 6 aa, Artificial Sequence
  Synthetic peptide
KVVYIQ

SEQ ID NO: 130 - protein, 6 aa, Artificial Sequence
  Synthetic peptide
VQKYIV

SEQ ID NO: 131 - protein, 6 aa, Artificial Sequence
  Synthetic peptide
VQKYVI

SEQ ID NO: 132 - protein, 6 aa, Artificial Sequence
  Synthetic peptide
KVVQIY

SEQ ID NO: 133 - protein, 6 aa, Artificial Sequence
  Synthetic peptide
YVIKIY

SEQ ID NO: 134 - protein, 6 aa, Artificial Sequence
  Synthetic peptide
QVIVYV

SEQ ID NO: 135 - protein, 6 aa, Artificial Sequence
  Synthetic peptide
VVIKVY

SEQ ID NO: 136 - protein, 6 aa, Artificial Sequence
  Synthetic peptide
KVVIYI

SEQ ID NO: 137 - protein, 6 aa, Artificial Sequence
  Synthetic peptide
VYVVIK

SEQ ID NO: 138 - protein, 6 aa, Artificial Sequence
  Synthetic peptide
YIVQVY

SEQ ID NO: 139 - protein, 6 aa, Artificial Sequence
  Synthetic peptide
YVIQVY

SEQ ID NO: 140 - protein, 6 aa, Artificial Sequence
  Synthetic peptide
YVIVVY

SEQ ID NO: 141 - protein, 6 aa, Artificial Sequence
  Synthetic peptide
KVIVYI

SEQ ID NO: 142 - protein, 6 aa, Artificial Sequence
  Synthetic peptide
YVIKVY

SEQ ID NO: 143 - protein, 6 aa, Artificial Sequence
  Synthetic peptide
VQVIVK

SEQ ID NO: 144 - protein, 6 aa, Artificial Sequence
  Synthetic peptide
YVVKIY

SEQ ID NO: 145 - protein, 6 aa, Artificial Sequence
  Synthetic peptide
YVVQVI

SEQ ID NO: 146 - protein, 6 aa, Artificial Sequence
  Synthetic peptide
QIVVYQ

SEQ ID NO: 147 - protein, 6 aa, Artificial Sequence
  Synthetic peptide
VVQIVK

SEQ ID NO: 148 - protein, 6 aa, Artificial Sequence
  Synthetic peptide
VQIVVK

SEQ ID NO: 149 - protein, 6 aa, Artificial Sequence
  Synthetic peptide
VIVVYK

SEQ ID NO: 150 - protein, 6 aa, Artificial Sequence
  Synthetic peptide
IVQVYI

SEQ ID NO: 151 - protein, 6 aa, Artificial Sequence
  Synthetic peptide
YVVIQV

SEQ ID NO: 152 - protein, 6 aa, Artificial Sequence
  Synthetic peptide
VYQVVI

SEQ ID NO: 153 - protein, 6 aa, Artificial Sequence
  Synthetic peptide
KYIQVY

SEQ ID NO: 154 - protein, 6 aa, Artificial Sequence
  Synthetic peptide
KYVVIY

SEQ ID NO: 155 - protein, 6 aa, Artificial Sequence
  Synthetic peptide
KYVQIY

SEQ ID NO: 156 - protein, 6 aa, Artificial Sequence
  Synthetic peptide
VVQVIK

SEQ ID NO: 157 - protein, 6 aa, Artificial Sequence
  Synthetic peptide
QKIVVK

SEQ ID NO: 158 - protein, 6 aa, Artificial Sequence
  Synthetic peptide
KYVIVY

SEQ ID NO: 159 - protein, 6 aa, Artificial Sequence
  Synthetic peptide
KQVVIK

SEQ ID NO: 160 - protein, 6 aa, Artificial Sequence
  Synthetic peptide
YVQIYV

SEQ ID NO: 161 - protein, 6 aa, Artificial Sequence
  Synthetic peptide
YKVQVY

SEQ ID NO: 162 - protein, 6 aa, Artificial Sequence
  Synthetic peptide
IKVQIY

SEQ ID NO: 163 - protein, 6 aa, Artificial Sequence
  Synthetic peptide
KIVVQK

SEQ ID NO: 164 - protein, 6 aa, Artificial Sequence
  Synthetic peptide
VIKVVI

SEQ ID NO: 165 - protein, 6 aa, Artificial Sequence
  Synthetic peptide
VYIKVV

SEQ ID NO: 166 - protein, 6 aa, Artificial Sequence
  Synthetic peptide
VYIVQV

SEQ ID NO: 167 - protein, 6 aa, Artificial Sequence
  Synthetic peptide
IVYVQI

SEQ ID NO: 168 - protein, 6 aa, Artificial Sequence
  Synthetic peptide
KVQIYK

SEQ ID NO: 169 - protein, 6 aa, Artificial Sequence
  Synthetic peptide
IYVIVY

SEQ ID NO: 170 - protein, 6 aa, Artificial Sequence
  Synthetic peptide
VIYVIV

SEQ ID NO: 171 - protein, 6 aa, Artificial Sequence
  Synthetic peptide
KYQVYI

SEQ ID NO: 172 - protein, 6 aa, Artificial Sequence
  Synthetic peptide
VIQKVV

SEQ ID NO: 173 - protein, 6 aa, Artificial Sequence
  Synthetic peptide
VKIVYV

SEQ ID NO: 174 - protein, 6 aa, Artificial Sequence
  Synthetic peptide
VKQIVV

SEQ ID NO: 175 - protein, 6 aa, Artificial Sequence
  Synthetic peptide
YIVKQY

SEQ ID NO: 176 - protein, 6 aa, Artificial Sequence
  Synthetic peptide
YIVVQY

SEQ ID NO: 177 - protein, 6 aa, Artificial Sequence
  Synthetic peptide
YKIQVY

SEQ ID NO: 178 - protein, 6 aa, Artificial Sequence
  Synthetic peptide
YQVVIY

SEQ ID NO: 179 - protein, 6 aa, Artificial Sequence
  Synthetic peptide
YVVKQY

SEQ ID NO: 180 - protein, 6 aa, Artificial Sequence
  Synthetic peptide
VIVKVQ

SEQ ID NO: 181 - protein, 6 aa, Artificial Sequence
  Synthetic peptide
QKVVIK

SEQ ID NO: 182 - protein, 6 aa, Artificial Sequence
  Synthetic peptide
GMVVVG

SEQ ID NO: 183 - protein, 6 aa, Artificial Sequence
  Synthetic peptide
GVVVMG

SEQ ID NO: 184 - protein, 6 aa, Artificial Sequence
  Synthetic peptide
GVVMVG

SEQ ID NO: 185 - protein, 6 aa, Artificial Sequence
  Synthetic peptide
GGVVVM

SEQ ID NO: 186 - protein, 6 aa, Artificial Sequence
  Synthetic peptide
GVVGVM

SEQ ID NO: 187 - protein, 6 aa, Artificial Sequence
  Synthetic peptide
GVVVGM

SEQ ID NO: 188 - protein, 6 aa, Artificial Sequence
  Synthetic peptide
GVMVVG

SEQ ID NO: 189 - protein, 6 aa, Artificial Sequence
  Synthetic peptide
GGVVMV

SEQ ID NO: 190 - protein, 6 aa, Artificial Sequence
  Synthetic peptide
GGVMVV

SEQ ID NO: 191 - protein, 6 aa, Artificial Sequence
  Synthetic peptide
GMVVGV

SEQ ID NO: 192 - protein, 6 aa, Artificial Sequence
  Synthetic peptide
GVVMGV

SEQ ID NO: 193 - protein, 6 aa, Artificial Sequence
  Synthetic peptide
GMVGVV

SEQ ID NO: 194 - protein, 6 aa, Artificial Sequence
  Synthetic peptide
GVMVGV

SEQ ID NO: 195 - protein, 6 aa, Artificial Sequence
  Synthetic peptide
GVVGMV

SEQ ID NO: 196 - protein, 6 aa, Artificial Sequence
  Synthetic peptide
GGMVVV

SEQ ID NO: 197 - protein, 6 aa, Artificial Sequence
  Synthetic peptide
GVGVMV

SEQ ID NO: 198 - protein, 6 aa, Artificial Sequence
  Synthetic peptide
GVGVVM

SEQ ID NO: 199 - protein, 6 aa, Artificial Sequence
  Synthetic peptide
MVVVGM

SEQ ID NO: 200 - protein, 6 aa, Artificial Sequence
  Synthetic peptide
GMGVVV

SEQ ID NO: 201 - protein, 6 aa, Artificial Sequence
  Synthetic peptide
MVVVGG

SEQ ID NO: 202 - protein, 6 aa, Artificial Sequence
  Synthetic peptide
GVGMVV

SEQ ID NO: 203 - protein, 6 aa, Artificial Sequence
  Synthetic peptide
GVVGGV

SEQ ID NO: 204 - protein, 6 aa, Artificial Sequence
  Synthetic peptide
MVGVVG

SEQ ID NO: 205 - protein, 6 aa, Artificial Sequence
  Synthetic peptide
VVMVGG

SEQ ID NO: 206 - protein, 6 aa, Artificial Sequence
  Synthetic peptide
MGVVVG

SEQ ID NO: 207 - protein, 6 aa, Artificial Sequence
  Synthetic peptide
MVGVGV

SEQ ID NO: 208 - protein, 6 aa, Artificial Sequence
  Synthetic peptide
VGVMVG

SEQ ID NO: 209 - protein, 6 aa, Artificial Sequence
  Synthetic peptide
MVVGVG

SEQ ID NO: 210 - protein, 6 aa, Artificial Sequence
  Synthetic peptide
VVGVMG

SEQ ID NO: 211 - protein, 6 aa, Artificial Sequence
  Synthetic peptide
VMGVVG

SEQ ID NO: 212 - protein, 6 aa, Artificial Sequence
  Synthetic peptide
VGVGVM

SEQ ID NO: 213 - protein, 6 aa, Artificial Sequence
  Synthetic peptide
VMVGVG

SEQ ID NO: 214 - protein, 6 aa, Artificial Sequence
  Synthetic peptide
MGGVVV

SEQ ID NO: 215 - protein, 6 aa, Artificial Sequence
  Synthetic peptide
VGMVVG

SEQ ID NO: 216 - protein, 6 aa, Artificial Sequence
  Synthetic peptide
VVGVGM

SEQ ID NO: 217 - protein, 6 aa, Artificial Sequence
  Synthetic peptide
VGGVVM

SEQ ID NO: 218 - protein, 6 aa, Artificial Sequence
  Synthetic peptide
VVGMVG

SEQ ID NO: 219 - protein, 6 aa, Artificial Sequence
  Synthetic peptide
VGVVMG

SEQ ID NO: 220 - protein, 6 aa, Artificial Sequence
  Synthetic peptide
MVGGVV

SEQ ID NO: 221 - protein, 6 aa, Artificial Sequence
  Synthetic peptide
VGMVGV

SEQ ID NO: 222 - protein, 6 aa, Artificial Sequence
  Synthetic peptide
VMVVGG

SEQ ID NO: 223 - protein, 6 aa, Artificial Sequence
  Synthetic peptide
VVGGVM

SEQ ID NO: 224 - protein, 6 aa, Artificial Sequence
  Synthetic peptide
VGGVMV

SEQ ID NO: 225 - protein, 6 aa, Artificial Sequence
  Synthetic peptide
VVVGMG

SEQ ID NO: 226 - protein, 6 aa, Artificial Sequence
  Synthetic peptide
VVGMGV

SEQ ID NO: 227 - protein, 6 aa, Artificial Sequence
  Synthetic peptide
MGVGVV

SEQ ID NO: 228 - protein, 6 aa, Artificial Sequence
  Synthetic peptide
VGVVGM

SEQ ID NO: 229 - protein, 6 aa, Artificial Sequence
  Synthetic peptide
VGGMVV

SEQ ID NO: 230 - protein, 6 aa, Artificial Sequence
  Synthetic peptide
VVVMGG

SEQ ID NO: 231 - protein, 6 aa, Artificial Sequence
  Synthetic peptide
VVMGGV

SEQ ID NO: 232 - protein, 6 aa, Artificial Sequence
  Synthetic peptide
VVVGGM

SEQ ID NO: 233 - protein, 6 aa, Artificial Sequence
  Synthetic peptide
VVGGMV

SEQ ID NO: 234 - protein, 6 aa, Artificial Sequence
  Synthetic peptide
VGMGVV

SEQ ID NO: 235 - protein, 6 aa, Artificial Sequence
  Synthetic peptide
VMGGVV

SEQ ID NO: 236 - protein, 6 aa, Artificial Sequence
  Synthetic peptide
VGGVGV

SEQ ID NO: 237 - protein, 6 aa, Artificial Sequence
  Synthetic peptide
VGVMGV

SEQ ID NO: 238 - protein, 6 aa, Artificial Sequence
  Synthetic peptide
VGVGMV

SEQ ID NO: 239 - protein, 6 aa, Artificial Sequence
  Synthetic peptide
VMVGGV

SEQ ID NO: 240 - protein, 6 aa, Artificial Sequence
  Synthetic peptide
ANSTSV

SEQ ID NO: 241 - protein, 6 aa, Artificial Sequence
  Synthetic peptide
ANSVSG

SEQ ID NO: 242 - protein, 6 aa, Artificial Sequence
  Synthetic peptide
ANSVSS

SEQ ID NO: 243 - protein, 6 aa, Artificial Sequence
  Synthetic peptide
AQNSNV

SEQ ID NO: 244 - protein, 6 aa, Artificial Sequence
  Synthetic peptide
AQNVNS

SEQ ID NO: 245 - protein, 6 aa, Artificial Sequence
  Synthetic peptide
AQNVTS

SEQ ID NO: 246 - protein, 6 aa, Artificial Sequence
  Synthetic peptide
AQSQSV

SEQ ID NO: 247 - protein, 6 aa, Artificial Sequence
  Synthetic peptide
AQSSSV

SEQ ID NO: 248 - protein, 6 aa, Artificial Sequence
  Synthetic peptide
AQSTSV

SEQ ID NO: 249 - protein, 6 aa, Artificial Sequence
  Synthetic peptide
AQSVNS

SEQ ID NO: 250 - protein, 6 aa, Artificial Sequence
  Synthetic peptide
AQSVQS

SEQ ID NO: 251 - protein, 6 aa, Artificial Sequence
  Synthetic peptide
AQSVSQ

SEQ ID NO: 252 - protein, 6 aa, Artificial Sequence
  Synthetic peptide
AQSVSS

SEQ ID NO: 253 - protein, 6 aa, Artificial Sequence
  Synthetic peptide
AQSVST

SEQ ID NO: 254 - protein, 6 aa, Artificial Sequence
  Synthetic peptide
ASNNNV

SEQ ID NO: 255 - protein, 6 aa, Artificial Sequence
  Synthetic peptide
ASNQNQ

SEQ ID NO: 256 - protein, 6 aa, Artificial Sequence
  Synthetic peptide
ASNQNV

SEQ ID NO: 257 - protein, 6 aa, Artificial Sequence
  Synthetic peptide
ASNQTQ

SEQ ID NO: 258 - protein, 6 aa, Artificial Sequence
  Synthetic peptide
ASNSNV

SEQ ID NO: 259 - protein, 6 aa, Artificial Sequence
  Synthetic peptide
ASNSTV

SEQ ID NO: 260 - protein, 6 aa, Artificial Sequence
  Synthetic peptide
ASNTNS

SEQ ID NO: 261 - protein, 6 aa, Artificial Sequence
  Synthetic peptide
ASNTNV

SEQ ID NO: 262 - protein, 6 aa, Artificial Sequence
  Synthetic peptide
ASNTSV

SEQ ID NO: 263 - protein, 6 aa, Artificial Sequence
  Synthetic peptide
ASNVNG

SEQ ID NO: 264 - protein, 6 aa, Artificial Sequence
  Synthetic peptide
ASNVNQ

SEQ ID NO: 265 - protein, 6 aa, Artificial Sequence
  Synthetic peptide
ASNVNS

SEQ ID NO: 266 - protein, 6 aa, Artificial Sequence
  Synthetic peptide
ASNVNT

SEQ ID NO: 267 - protein, 6 aa, Artificial Sequence
  Synthetic peptide
ASNVTG

SEQ ID NO: 268 - protein, 6 aa, Artificial Sequence
  Synthetic peptide
ASNVTT

SEQ ID NO: 269 - protein, 6 aa, Artificial Sequence
  Synthetic peptide
ASSNSV

SEQ ID NO: 270 - protein, 6 aa, Artificial Sequence
  Synthetic peptide
ASSVSG

SEQ ID NO: 271 - protein, 6 aa, Artificial Sequence
  Synthetic peptide
ASSVSN

SEQ ID NO: 272 - protein, 6 aa, Artificial Sequence
  Synthetic peptide
ATNVNS

SEQ ID NO: 273 - protein, 6 aa, Artificial Sequence
  Synthetic peptide
ATNVTS

SEQ ID NO: 274 - protein, 6 aa, Artificial Sequence
  Synthetic peptide
ATSQSQ

SEQ ID NO: 275 - protein, 6 aa, Artificial Sequence
  Synthetic peptide
ATSQSV

SEQ ID NO: 276 - protein, 6 aa, Artificial Sequence
  Synthetic peptide
ATSTSG

SEQ ID NO: 277 - protein, 6 aa, Artificial Sequence
  Synthetic peptide
ATSTSV

SEQ ID NO: 278 - protein, 6 aa, Artificial Sequence
  Synthetic peptide
ATSVSG

SEQ ID NO: 279 - protein, 6 aa, Artificial Sequence
  Synthetic peptide
ATSVSS

SEQ ID NO: 280 - protein, 6 aa, Artificial Sequence
  Synthetic peptide
AVNQNS

SEQ ID NO: 281 - protein, 6 aa, Artificial Sequence
  Synthetic peptide
AVNQSQ

SEQ ID NO: 282 - protein, 6 aa, Artificial Sequence
  Synthetic peptide
AVNSNG

SEQ ID NO: 283 - protein, 6 aa, Artificial Sequence
  Synthetic peptide
AVNSNS

SEQ ID NO: 284 - protein, 6 aa, Artificial Sequence
  Synthetic peptide
AVNSNT

SEQ ID NO: 285 - protein, 6 aa, Artificial Sequence
  Synthetic peptide
AVNTNS

SEQ ID NO: 286 - protein, 6 aa, Artificial Sequence
  Synthetic peptide
AVSNSG

SEQ ID NO: 287 - protein, 6 aa, Artificial Sequence
  Synthetic peptide
AVSNSS

SEQ ID NO: 288 - protein, 6 aa, Artificial Sequence
  Synthetic peptide
AVSQNQ

SEQ ID NO: 289 - protein, 6 aa, Artificial Sequence
  Synthetic peptide
AVSQSG

SEQ ID NO: 290 - protein, 6 aa, Artificial Sequence
  Synthetic peptide
AVSQSQ

SEQ ID NO: 291 - protein, 6 aa, Artificial Sequence
  Synthetic peptide
AVSQTQ

SEQ ID NO: 292 - protein, 6 aa, Artificial Sequence
  Synthetic peptide
AVSSNQ

SEQ ID NO: 293 - protein, 6 aa, Artificial Sequence
  Synthetic peptide
AVSSNS

SEQ ID NO: 294 - protein, 6 aa, Artificial Sequence
  Synthetic peptide
AVSSSQ

SEQ ID NO: 295 - protein, 6 aa, Artificial Sequence
  Synthetic peptide
AVSTSG

SEQ ID NO: 296 - protein, 6 aa, Artificial Sequence
  Synthetic peptide
GANTVS

SEQ ID NO: 297 - protein, 6 aa, Artificial Sequence
  Synthetic peptide
GAQTSS

SEQ ID NO: 298 - protein, 6 aa, Artificial Sequence
  Synthetic peptide
GASNQS

SEQ ID NO: 299 - protein, 6 aa, Artificial Sequence
  Synthetic peptide
GASQQS

SEQ ID NO: 300 - protein, 6 aa, Artificial Sequence
  Synthetic peptide
GASSQQ

SEQ ID NO: 301 - protein, 6 aa, Artificial Sequence
  Synthetic peptide
GGQVTS

SEQ ID NO: 302 - protein, 6 aa, Artificial Sequence
  Synthetic peptide
GGSNQV

SEQ ID NO: 303 - protein, 6 aa, Artificial Sequence
  Synthetic peptide
GNNVQS

SEQ ID NO: 304 - protein, 6 aa, Artificial Sequence
  Synthetic peptide
GNQVTS

SEQ ID NO: 305 - protein, 6 aa, Artificial Sequence
  Synthetic peptide
GNSNQV

SEQ ID NO: 306 - protein, 6 aa, Artificial Sequence
  Synthetic peptide
GNSQQQ

SEQ ID NO: 307 - protein, 6 aa, Artificial Sequence
  Synthetic peptide
GNSQQS

SEQ ID NO: 308 - protein, 6 aa, Artificial Sequence
  Synthetic peptide
GNSQQV

SEQ ID NO: 309 - protein, 6 aa, Artificial Sequence
  Synthetic peptide
GNSSTV

SEQ ID NO: 310 - protein, 6 aa, Artificial Sequence
  Synthetic peptide
GNSTQS

SEQ ID NO: 311 - protein, 6 aa, Artificial Sequence
  Synthetic peptide
GNSTQV

SEQ ID NO: 312 - protein, 6 aa, Artificial Sequence
  Synthetic peptide
GNSTVS

SEQ ID NO: 313 - protein, 6 aa, Artificial Sequence
  Synthetic peptide
GNSVQS

SEQ ID NO: 314 - protein, 6 aa, Artificial Sequence
  Synthetic peptide
GNSVSS

SEQ ID NO: 315 - protein, 6 aa, Artificial Sequence
  Synthetic peptide
GNSVST

SEQ ID NO: 316 - protein, 6 aa, Artificial Sequence
  Synthetic peptide
GNSVTS

SEQ ID NO: 317 - protein, 6 aa, Artificial Sequence
  Synthetic peptide
GQNTVS

SEQ ID NO: 318 - protein, 6 aa, Artificial Sequence
  Synthetic peptide
GQNVAS

SEQ ID NO: 319 - protein, 6 aa, Artificial Sequence
  Synthetic peptide
GQNVQS

SEQ ID NO: 320 - protein, 6 aa, Artificial Sequence
  Synthetic peptide
GQNVSS

SEQ ID NO: 321 - protein, 6 aa, Artificial Sequence
  Synthetic peptide
GQNVTS

SEQ ID NO: 322 - protein, 6 aa, Artificial Sequence
  Synthetic peptide
GQQQSQ

SEQ ID NO: 323 - protein, 6 aa, Artificial Sequence
  Synthetic peptide
GQQTSS

SEQ ID NO: 324 - protein, 6 aa, Artificial Sequence
  Synthetic peptide
GQQTSV

SEQ ID NO: 325 - protein, 6 aa, Artificial Sequence
  Synthetic peptide
GQQVAS

SEQ ID NO: 326 - protein, 6 aa, Artificial Sequence
  Synthetic peptide
GQQVNS

SEQ ID NO: 327 - protein, 6 aa, Artificial Sequence
  Synthetic peptide
GQQVQS

SEQ ID NO: 328 - protein, 6 aa, Artificial Sequence
  Synthetic peptide
GQQVSG

SEQ ID NO: 329 - protein, 6 aa, Artificial Sequence
  Synthetic peptide
GQQVSQ

SEQ ID NO: 330 - protein, 6 aa, Artificial Sequence
  Synthetic peptide
GQQVSS

SEQ ID NO: 331 - protein, 6 aa, Artificial Sequence
  Synthetic peptide
GQQVST

SEQ ID NO: 332 - protein, 6 aa, Artificial Sequence
  Synthetic peptide
GQQVTS

SEQ ID NO: 333 - protein, 6 aa, Artificial Sequence
  Synthetic peptide
GQSGQV

SEQ ID NO: 334 - protein, 6 aa, Artificial Sequence
  Synthetic peptide
GQSNQA

SEQ ID NO: 335 - protein, 6 aa, Artificial Sequence
  Synthetic peptide
GQSNQS

SEQ ID NO: 336 - protein, 6 aa, Artificial Sequence
  Synthetic peptide
GQSNQV

SEQ ID NO: 337 - protein, 6 aa, Artificial Sequence
  Synthetic peptide
GQSQAQ

SEQ ID NO: 338 - protein, 6 aa, Artificial Sequence
  Synthetic peptide
GQSQQQ

SEQ ID NO: 339 - protein, 6 aa, Artificial Sequence
  Synthetic peptide
GQSQQS

SEQ ID NO: 340 - protein, 6 aa, Artificial Sequence
  Synthetic peptide
GQSQSQ

SEQ ID NO: 341 - protein, 6 aa, Artificial Sequence
  Synthetic peptide
GQSSQQ

SEQ ID NO: 342 - protein, 6 aa, Artificial Sequence
  Synthetic peptide
GQSSQS

SEQ ID NO: 343 - protein, 6 aa, Artificial Sequence
  Synthetic peptide
GQSSQV

SEQ ID NO: 344 - protein, 6 aa, Artificial Sequence
  Synthetic peptide
GQSTQS

SEQ ID NO: 345 - protein, 6 aa, Artificial Sequence
  Synthetic peptide
GQSTQV

SEQ ID NO: 346 - protein, 6 aa, Artificial Sequence
  Synthetic peptide
GQSVAG

SEQ ID NO: 347 - protein, 6 aa, Artificial Sequence
  Synthetic peptide
GQSVAQ

SEQ ID NO: 348 - protein, 6 aa, Artificial Sequence
  Synthetic peptide
GQSVAS

SEQ ID NO: 349 - protein, 6 aa, Artificial Sequence
  Synthetic peptide
GQSVQN

SEQ ID NO: 350 - protein, 6 aa, Artificial Sequence
  Synthetic peptide
GQSVQQ

SEQ ID NO: 351 - protein, 6 aa, Artificial Sequence
  Synthetic peptide
GQSVQS

SEQ ID NO: 352 - protein, 6 aa, Artificial Sequence
  Synthetic peptide
GQSVSG

SEQ ID NO: 353 - protein, 6 aa, Artificial Sequence
  Synthetic peptide
GQSVSN

SEQ ID NO: 354 - protein, 6 aa, Artificial Sequence
  Synthetic peptide
GQSVSQ

SEQ ID NO: 355 - protein, 6 aa, Artificial Sequence
  Synthetic peptide
GQSVSS

SEQ ID NO: 356 - protein, 6 aa, Artificial Sequence
  Synthetic peptide
GQSVST

SEQ ID NO: 357 - protein, 6 aa, Artificial Sequence
  Synthetic peptide
GQSVTS

SEQ ID NO: 358 - protein, 6 aa, Artificial Sequence
  Synthetic peptide
GSNQQV

SEQ ID NO: 359 - protein, 6 aa, Artificial Sequence
  Synthetic peptide
GSNQVQ

SEQ ID NO: 360 - protein, 6 aa, Artificial Sequence
  Synthetic peptide
GSNSTV

SEQ ID NO: 361 - protein, 6 aa, Artificial Sequence
  Synthetic peptide
GSNSVQ

SEQ ID NO: 362 - protein, 6 aa, Artificial Sequence
  Synthetic peptide
GSNSVT

SEQ ID NO: 363 - protein, 6 aa, Artificial Sequence
  Synthetic peptide
GSNTAV

SEQ ID NO: 364 - protein, 6 aa, Artificial Sequence
  Synthetic peptide
GSNTQV

SEQ ID NO: 365 - protein, 6 aa, Artificial Sequence
  Synthetic peptide
GSNTVA

SEQ ID NO: 366 - protein, 6 aa, Artificial Sequence
  Synthetic peptide
GSNTVS

SEQ ID NO: 367 - protein, 6 aa, Artificial Sequence
  Synthetic peptide
GSNVAS

SEQ ID NO: 368 - protein, 6 aa, Artificial Sequence
  Synthetic peptide
GSNVQQ

SEQ ID NO: 369 - protein, 6 aa, Artificial Sequence
  Synthetic peptide
GSNVQS

SEQ ID NO: 370 - protein, 6 aa, Artificial Sequence
  Synthetic peptide
GSNVQT

SEQ ID NO: 371 - protein, 6 aa, Artificial Sequence
  Synthetic peptide
GSNVTS

SEQ ID NO: 372 - protein, 6 aa, Artificial Sequence
  Synthetic peptide
GSQQSV

SEQ ID NO: 373 - protein, 6 aa, Artificial Sequence
  Synthetic peptide
GSQQTQ

SEQ ID NO: 374 - protein, 6 aa, Artificial Sequence
  Synthetic peptide
GSQQTV

SEQ ID NO: 375 - protein, 6 aa, Artificial Sequence
  Synthetic peptide
GSQTSS

SEQ ID NO: 376 - protein, 6 aa, Artificial Sequence
  Synthetic peptide
GSQTSV

SEQ ID NO: 377 - protein, 6 aa, Artificial Sequence
  Synthetic peptide
GSQVAS

SEQ ID NO: 378 - protein, 6 aa, Artificial Sequence
  Synthetic peptide
GSQVNS

SEQ ID NO: 379 - protein, 6 aa, Artificial Sequence
  Synthetic peptide
GSQVQS

SEQ ID NO: 380 - protein, 6 aa, Artificial Sequence
  Synthetic peptide
GSQVSS

SEQ ID NO: 381 - protein, 6 aa, Artificial Sequence
  Synthetic peptide
GSQVST

SEQ ID NO: 382 - protein, 6 aa, Artificial Sequence
  Synthetic peptide
GSQVTG

SEQ ID NO: 383 - protein, 6 aa, Artificial Sequence
  Synthetic peptide
GSQVTS

SEQ ID NO: 384 - protein, 6 aa, Artificial Sequence
  Synthetic peptide
GSSAQS

SEQ ID NO: 385 - protein, 6 aa, Artificial Sequence
  Synthetic peptide
GSSGQV

SEQ ID NO: 386 - protein, 6 aa, Artificial Sequence
  Synthetic peptide
GSSNQA

SEQ ID NO: 387 - protein, 6 aa, Artificial Sequence
  Synthetic peptide
GSSNQT

SEQ ID NO: 388 - protein, 6 aa, Artificial Sequence
  Synthetic peptide
GSSNQV

SEQ ID NO: 389 - protein, 6 aa, Artificial Sequence
  Synthetic peptide
GSSNSV

SEQ ID NO: 390 - protein, 6 aa, Artificial Sequence
  Synthetic peptide
GSSNTV

SEQ ID NO: 391 - protein, 6 aa, Artificial Sequence
  Synthetic peptide
GSSNVT

SEQ ID NO: 392 - protein, 6 aa, Artificial Sequence
  Synthetic peptide
GSSQAQ

SEQ ID NO: 393 - protein, 6 aa, Artificial Sequence
  Synthetic peptide
GSSQQA

SEQ ID NO: 394 - protein, 6 aa, Artificial Sequence
  Synthetic peptide
GSSQQV

SEQ ID NO: 395 - protein, 6 aa, Artificial Sequence
  Synthetic peptide
GSSQSQ

SEQ ID NO: 396 - protein, 6 aa, Artificial Sequence
  Synthetic peptide
GSSSQQ

SEQ ID NO: 397 - protein, 6 aa, Artificial Sequence
  Synthetic peptide
GSSSQS

SEQ ID NO: 398 - protein, 6 aa, Artificial Sequence
  Synthetic peptide
GSSSQV

SEQ ID NO: 399 - protein, 6 aa, Artificial Sequence
  Synthetic peptide
GSSTQA

SEQ ID NO: 400 - protein, 6 aa, Artificial Sequence
  Synthetic peptide
GSSTQG

SEQ ID NO: 401 - protein, 6 aa, Artificial Sequence
  Synthetic peptide
GSSTQT

SEQ ID NO: 402 - protein, 6 aa, Artificial Sequence
  Synthetic peptide
GSSTQV

SEQ ID NO: 403 - protein, 6 aa, Artificial Sequence
  Synthetic peptide
GSSTSV

SEQ ID NO: 404 - protein, 6 aa, Artificial Sequence
  Synthetic peptide
GSSTVN

SEQ ID NO: 405 - protein, 6 aa, Artificial Sequence
  Synthetic peptide
GSSVNS

SEQ ID NO: 406 - protein, 6 aa, Artificial Sequence
  Synthetic peptide
GSSVQN

SEQ ID NO: 407 - protein, 6 aa, Artificial Sequence
  Synthetic peptide
GSSVQT

SEQ ID NO: 408 - protein, 6 aa, Artificial Sequence
  Synthetic peptide
GSSVSG

SEQ ID NO: 409 - protein, 6 aa, Artificial Sequence
  Synthetic peptide
GSSVSS

SEQ ID NO: 410 - protein, 6 aa, Artificial Sequence
  Synthetic peptide
GSSVST

SEQ ID NO: 411 - protein, 6 aa, Artificial Sequence
  Synthetic peptide
GSSVTN

SEQ ID NO: 412 - protein, 6 aa, Artificial Sequence
  Synthetic peptide
GTNSSV

SEQ ID NO: 413 - protein, 6 aa, Artificial Sequence
  Synthetic peptide
GTNSVS

SEQ ID NO: 414 - protein, 6 aa, Artificial Sequence
  Synthetic peptide
GTNTVS

SEQ ID NO: 415 - protein, 6 aa, Artificial Sequence
  Synthetic peptide
GTNVQS

SEQ ID NO: 416 - protein, 6 aa, Artificial Sequence
  Synthetic peptide
GTNVSS

SEQ ID NO: 417 - protein, 6 aa, Artificial Sequence
  Synthetic peptide
GTNVTS

SEQ ID NO: 418 - protein, 6 aa, Artificial Sequence
  Synthetic peptide
GTQQSQ

SEQ ID NO: 419 - protein, 6 aa, Artificial Sequence
  Synthetic peptide
GTQSTS

SEQ ID NO: 420 - protein, 6 aa, Artificial Sequence
  Synthetic peptide
GTQSTV

SEQ ID NO: 421 - protein, 6 aa, Artificial Sequence
  Synthetic peptide
GTQTSA

SEQ ID NO: 422 - protein, 6 aa, Artificial Sequence
  Synthetic peptide
GTQTSV

SEQ ID NO: 423 - protein, 6 aa, Artificial Sequence
  Synthetic peptide
GTQVQS

SEQ ID NO: 424 - protein, 6 aa, Artificial Sequence
  Synthetic peptide
GTQVSG

SEQ ID NO: 425 - protein, 6 aa, Artificial Sequence
  Synthetic peptide
GTQVSN

SEQ ID NO: 426 - protein, 6 aa, Artificial Sequence
  Synthetic peptide
GTQVSS

SEQ ID NO: 427 - protein, 6 aa, Artificial Sequence
  Synthetic peptide
GTQVST

SEQ ID NO: 428 - protein, 6 aa, Artificial Sequence
  Synthetic peptide
GTSAQS

SEQ ID NO: 429 - protein, 6 aa, Artificial Sequence
  Synthetic peptide
GTSGQV

SEQ ID NO: 430 - protein, 6 aa, Artificial Sequence
  Synthetic peptide
GTSNQA

SEQ ID NO: 431 - protein, 6 aa, Artificial Sequence
  Synthetic peptide
GTSNQS

SEQ ID NO: 432 - protein, 6 aa, Artificial Sequence
  Synthetic peptide
GTSNQT

SEQ ID NO: 433 - protein, 6 aa, Artificial Sequence
  Synthetic peptide
GTSNQV

SEQ ID NO: 434 - protein, 6 aa, Artificial Sequence
  Synthetic peptide
GTSNSV

SEQ ID NO: 435 - protein, 6 aa, Artificial Sequence
  Synthetic peptide
GTSNVS

SEQ ID NO: 436 - protein, 6 aa, Artificial Sequence
  Synthetic peptide
GTSQQA

SEQ ID NO: 437 - protein, 6 aa, Artificial Sequence
  Synthetic peptide
GTSQQS

SEQ ID NO: 438 - protein, 6 aa, Artificial Sequence
  Synthetic peptide
GTSQQV

SEQ ID NO: 439 - protein, 6 aa, Artificial Sequence
  Synthetic peptide
GTSQSQ

SEQ ID NO: 440 - protein, 6 aa, Artificial Sequence
  Synthetic peptide
GTSQSV

SEQ ID NO: 441 - protein, 6 aa, Artificial Sequence
  Synthetic peptide
GTSSNV

SEQ ID NO: 442 - protein, 6 aa, Artificial Sequence
  Synthetic peptide
GTSTQA

SEQ ID NO: 443 - protein, 6 aa, Artificial Sequence
  Synthetic peptide
GTSTQS

SEQ ID NO: 444 - protein, 6 aa, Artificial Sequence
  Synthetic peptide
GTSTQT

SEQ ID NO: 445 - protein, 6 aa, Artificial Sequence
  Synthetic peptide
GTSTQV

SEQ ID NO: 446 - protein, 6 aa, Artificial Sequence
  Synthetic peptide
GTSTSV

SEQ ID NO: 447 - protein, 6 aa, Artificial Sequence
  Synthetic peptide
GTSVAG

SEQ ID NO: 448 - protein, 6 aa, Artificial Sequence
  Synthetic peptide
GTSVAS

SEQ ID NO: 449 - protein, 6 aa, Artificial Sequence
  Synthetic peptide
GTSVNS

SEQ ID NO: 450 - protein, 6 aa, Artificial Sequence
  Synthetic peptide
GTSVNT

SEQ ID NO: 451 - protein, 6 aa, Artificial Sequence
  Synthetic peptide
GTSVQG

SEQ ID NO: 452 - protein, 6 aa, Artificial Sequence
  Synthetic peptide
GTSVQN

SEQ ID NO: 453 - protein, 6 aa, Artificial Sequence
  Synthetic peptide
GTSVQQ

SEQ ID NO: 454 - protein, 6 aa, Artificial Sequence
  Synthetic peptide
GTSVQS

SEQ ID NO: 455 - protein, 6 aa, Artificial Sequence
  Synthetic peptide
GTSVQT

SEQ ID NO: 456 - protein, 6 aa, Artificial Sequence
  Synthetic peptide
GTSVSG

SEQ ID NO: 457 - protein, 6 aa, Artificial Sequence
  Synthetic peptide
GTSVSN

SEQ ID NO: 458 - protein, 6 aa, Artificial Sequence
  Synthetic peptide
GTSVSS

SEQ ID NO: 459 - protein, 6 aa, Artificial Sequence
  Synthetic peptide
GTSVST

SEQ ID NO: 460 - protein, 6 aa, Artificial Sequence
  Synthetic peptide
GTSVTS

SEQ ID NO: 461 - protein, 6 aa, Artificial Sequence
  Synthetic peptide
GVNSQS

SEQ ID NO: 462 - protein, 6 aa, Artificial Sequence
  Synthetic peptide
GVNSST

SEQ ID NO: 463 - protein, 6 aa, Artificial Sequence
  Synthetic peptide
GVNSTS

SEQ ID NO: 464 - protein, 6 aa, Artificial Sequence
  Synthetic peptide
GVNTQS

SEQ ID NO: 465 - protein, 6 aa, Artificial Sequence
  Synthetic peptide
GVNTSS

SEQ ID NO: 466 - protein, 6 aa, Artificial Sequence
  Synthetic peptide
GVQNTS

SEQ ID NO: 467 - protein, 6 aa, Artificial Sequence
  Synthetic peptide
GVQQSQ

SEQ ID NO: 468 - protein, 6 aa, Artificial Sequence
  Synthetic peptide
GVQSNQ

SEQ ID NO: 469 - protein, 6 aa, Artificial Sequence
  Synthetic peptide
GVQSNS

SEQ ID NO: 470 - protein, 6 aa, Artificial Sequence
  Synthetic peptide
GVQSSG

SEQ ID NO: 471 - protein, 6 aa, Artificial Sequence
  Synthetic peptide
GVQSSQ

SEQ ID NO: 472 - protein, 6 aa, Artificial Sequence
  Synthetic peptide
GVQSTS

SEQ ID NO: 473 - protein, 6 aa, Artificial Sequence
  Synthetic peptide
GVQSTT

SEQ ID NO: 474 - protein, 6 aa, Artificial Sequence
  Synthetic peptide
GVQTNS

SEQ ID NO: 475 - protein, 6 aa, Artificial Sequence
  Synthetic peptide
GVQTQS

SEQ ID NO: 476 - protein, 6 aa, Artificial Sequence
  Synthetic peptide
GVQTSG

SEQ ID NO: 477 - protein, 6 aa, Artificial Sequence
  Synthetic peptide
GVQTSS

SEQ ID NO: 478 - protein, 6 aa, Artificial Sequence
  Synthetic peptide
GVSGQG

SEQ ID NO: 479 - protein, 6 aa, Artificial Sequence
  Synthetic peptide
GVSGQT

SEQ ID NO: 480 - protein, 6 aa, Artificial Sequence
  Synthetic peptide
GVSNAS

SEQ ID NO: 481 - protein, 6 aa, Artificial Sequence
  Synthetic peptide
GVSNNV

SEQ ID NO: 482 - protein, 6 aa, Artificial Sequence
  Synthetic peptide
GVSNQG

SEQ ID NO: 483 - protein, 6 aa, Artificial Sequence
  Synthetic peptide
GVSNQN

SEQ ID NO: 484 - protein, 6 aa, Artificial Sequence
  Synthetic peptide
GVSNQQ

SEQ ID NO: 485 - protein, 6 aa, Artificial Sequence
  Synthetic peptide
GVSNQS

SEQ ID NO: 486 - protein, 6 aa, Artificial Sequence
  Synthetic peptide
GVSNQT

SEQ ID NO: 487 - protein, 6 aa, Artificial Sequence
  Synthetic peptide
GVSNSS

SEQ ID NO: 488 - protein, 6 aa, Artificial Sequence
  Synthetic peptide
GVSNST

SEQ ID NO: 489 - protein, 6 aa, Artificial Sequence
  Synthetic peptide
GVSNTS

SEQ ID NO: 490 - protein, 6 aa, Artificial Sequence
  Synthetic peptide
GVSQAQ

SEQ ID NO: 491 - protein, 6 aa, Artificial Sequence
  Synthetic peptide
GVSQNQ

SEQ ID NO: 492 - protein, 6 aa, Artificial Sequence
  Synthetic peptide
GVSQNS

SEQ ID NO: 493 - protein, 6 aa, Artificial Sequence
  Synthetic peptide
GVSQQG

SEQ ID NO: 494 - protein, 6 aa, Artificial Sequence
  Synthetic peptide
GVSQQS

SEQ ID NO: 495 - protein, 6 aa, Artificial Sequence
  Synthetic peptide
GVSQQT

SEQ ID NO: 496 - protein, 6 aa, Artificial Sequence
  Synthetic peptide
GVSQSG

SEQ ID NO: 497 - protein, 6 aa, Artificial Sequence
  Synthetic peptide
GVSQSQ

SEQ ID NO: 498 - protein, 6 aa, Artificial Sequence
  Synthetic peptide
GVSQSS

SEQ ID NO: 499 - protein, 6 aa, Artificial Sequence
  Synthetic peptide
GVSSAQ

SEQ ID NO: 500 - protein, 6 aa, Artificial Sequence
  Synthetic peptide
GVSSAS

SEQ ID NO: 501 - protein, 6 aa, Artificial Sequence
  Synthetic peptide
GVSSGQ

SEQ ID NO: 502 - protein, 6 aa, Artificial Sequence
  Synthetic peptide
GVSSNG

SEQ ID NO: 503 - protein, 6 aa, Artificial Sequence
  Synthetic peptide
GVSSNQ

SEQ ID NO: 504 - protein, 6 aa, Artificial Sequence
  Synthetic peptide
GVSSNS

SEQ ID NO: 505 - protein, 6 aa, Artificial Sequence
  Synthetic peptide
GVSSNT

SEQ ID NO: 506 - protein, 6 aa, Artificial Sequence
  Synthetic peptide
GVSSQG

SEQ ID NO: 507 - protein, 6 aa, Artificial Sequence
  Synthetic peptide
GVSSQN

SEQ ID NO: 508 - protein, 6 aa, Artificial Sequence
  Synthetic peptide
GVSSQQ

SEQ ID NO: 509 - protein, 6 aa, Artificial Sequence
  Synthetic peptide
GVSSSG

SEQ ID NO: 510 - protein, 6 aa, Artificial Sequence
  Synthetic peptide
GVSSSQ

SEQ ID NO: 511 - protein, 6 aa, Artificial Sequence
  Synthetic peptide
GVSSSS

SEQ ID NO: 512 - protein, 6 aa, Artificial Sequence
  Synthetic peptide
GVSSTN

SEQ ID NO: 513 - protein, 6 aa, Artificial Sequence
  Synthetic peptide
GVSTAS

SEQ ID NO: 514 - protein, 6 aa, Artificial Sequence
  Synthetic peptide
GVSTNS

SEQ ID NO: 515 - protein, 6 aa, Artificial Sequence
  Synthetic peptide
GVSTQG

SEQ ID NO: 516 - protein, 6 aa, Artificial Sequence
  Synthetic peptide
GVSTQN

SEQ ID NO: 517 - protein, 6 aa, Artificial Sequence
  Synthetic peptide
GVSTQQ

SEQ ID NO: 518 - protein, 6 aa, Artificial Sequence
  Synthetic peptide
GVSTQS

SEQ ID NO: 519 - protein, 6 aa, Artificial Sequence
  Synthetic peptide
GVSTQT

SEQ ID NO: 520 - protein, 6 aa, Artificial Sequence
  Synthetic peptide
GVSTSN

SEQ ID NO: 521 - protein, 6 aa, Artificial Sequence
  Synthetic peptide
GVSTSQ

SEQ ID NO: 522 - protein, 6 aa, Artificial Sequence
  Synthetic peptide
GVSTSS

SEQ ID NO: 523 - protein, 6 aa, Artificial Sequence
  Synthetic peptide
GVSTST

SEQ ID NO: 524 - protein, 6 aa, Artificial Sequence
  Synthetic peptide
GVTNSS

SEQ ID NO: 525 - protein, 6 aa, Artificial Sequence
  Synthetic peptide
GVTSNS

SEQ ID NO: 526 - protein, 6 aa, Artificial Sequence
  Synthetic peptide
GVTSSN

SEQ ID NO: 527 - protein, 6 aa, Artificial Sequence
  Synthetic peptide
NGSTSV

SEQ ID NO: 528 - protein, 6 aa, Artificial Sequence
  Synthetic peptide
NGSVTS

SEQ ID NO: 529 - protein, 6 aa, Artificial Sequence
  Synthetic peptide
NNSVSS

SEQ ID NO: 530 - protein, 6 aa, Artificial Sequence
  Synthetic peptide
NQSQSQ

SEQ ID NO: 531 - protein, 6 aa, Artificial Sequence
  Synthetic peptide
NQSVSN

SEQ ID NO: 532 - protein, 6 aa, Artificial Sequence
  Synthetic peptide
NQSVSQ

SEQ ID NO: 533 - protein, 6 aa, Artificial Sequence
  Synthetic peptide
NQSVSS

SEQ ID NO: 534 - protein, 6 aa, Artificial Sequence
  Synthetic peptide
NSSNSV

SEQ ID NO: 535 - protein, 6 aa, Artificial Sequence
  Synthetic peptide
NSSQSQ

SEQ ID NO: 536 - protein, 6 aa, Artificial Sequence
  Synthetic peptide
NSSTVG

SEQ ID NO: 537 - protein, 6 aa, Artificial Sequence
  Synthetic peptide
NSSVSG

SEQ ID NO: 538 - protein, 6 aa, Artificial Sequence
  Synthetic peptide
NSSVSN

SEQ ID NO: 539 - protein, 6 aa, Artificial Sequence
  Synthetic peptide
NSSVSS

SEQ ID NO: 540 - protein, 6 aa, Artificial Sequence
  Synthetic peptide
NSSVTG

SEQ ID NO: 541 - protein, 6 aa, Artificial Sequence
  Synthetic peptide
NTNVNS

SEQ ID NO: 542 - protein, 6 aa, Artificial Sequence
  Synthetic peptide
NTQVSS

SEQ ID NO: 543 - protein, 6 aa, Artificial Sequence
  Synthetic peptide
NTSGSV

SEQ ID NO: 544 - protein, 6 aa, Artificial Sequence
  Synthetic peptide
NTSQSQ

SEQ ID NO: 545 - protein, 6 aa, Artificial Sequence
  Synthetic peptide
NTSTSS

SEQ ID NO: 546 - protein, 6 aa, Artificial Sequence
  Synthetic peptide
NTSTSV

SEQ ID NO: 547 - protein, 6 aa, Artificial Sequence
  Synthetic peptide
NTSVSS

SEQ ID NO: 548 - protein, 6 aa, Artificial Sequence
  Synthetic peptide
NVSGST

SEQ ID NO: 549 - protein, 6 aa, Artificial Sequence
  Synthetic peptide
NVSNSG

SEQ ID NO: 550 - protein, 6 aa, Artificial Sequence
  Synthetic peptide
NVSQSG

SEQ ID NO: 551 - protein, 6 aa, Artificial Sequence
  Synthetic peptide
NVSQSQ

SEQ ID NO: 552 - protein, 6 aa, Artificial Sequence
  Synthetic peptide
NVSQSS

SEQ ID NO: 553 - protein, 6 aa, Artificial Sequence
  Synthetic peptide
NVSSSG

SEQ ID NO: 554 - protein, 6 aa, Artificial Sequence
  Synthetic peptide
NVSSSS

SEQ ID NO: 555 - protein, 6 aa, Artificial Sequence
  Synthetic peptide
NVSSTG

SEQ ID NO: 556 - protein, 6 aa, Artificial Sequence
  Synthetic peptide
NVSTSN

SEQ ID NO: 557 - protein, 6 aa, Artificial Sequence
  Synthetic peptide
NVSTSS

SEQ ID NO: 558 - protein, 6 aa, Artificial Sequence
  Synthetic peptide
NVSTST

SEQ ID NO: 559 - protein, 6 aa, Artificial Sequence
  Synthetic peptide
QNSTSV

SEQ ID NO: 560 - protein, 6 aa, Artificial Sequence
  Synthetic peptide
QNSVSG

SEQ ID NO: 561 - protein, 6 aa, Artificial Sequence
  Synthetic peptide
QQSQSQ

SEQ ID NO: 562 - protein, 6 aa, Artificial Sequence
  Synthetic peptide
QQSVAG

SEQ ID NO: 563 - protein, 6 aa, Artificial Sequence
  Synthetic peptide
QQSVAQ

SEQ ID NO: 564 - protein, 6 aa, Artificial Sequence
  Synthetic peptide
QQSVAS

SEQ ID NO: 565 - protein, 6 aa, Artificial Sequence
  Synthetic peptide
QQSVAT

SEQ ID NO: 566 - protein, 6 aa, Artificial Sequence
  Synthetic peptide
QQSVSG

SEQ ID NO: 567 - protein, 6 aa, Artificial Sequence
  Synthetic peptide
QQSVSN

SEQ ID NO: 568 - protein, 6 aa, Artificial Sequence
  Synthetic peptide
QQSVSS

SEQ ID NO: 569 - protein, 6 aa, Artificial Sequence
  Synthetic peptide
QSNQVQ

SEQ ID NO: 570 - protein, 6 aa, Artificial Sequence
  Synthetic peptide
QSNTAV

SEQ ID NO: 571 - protein, 6 aa, Artificial Sequence
  Synthetic peptide
QSNTNV

SEQ ID NO: 572 - protein, 6 aa, Artificial Sequence
  Synthetic peptide
QSSNSV

SEQ ID NO: 573 - protein, 6 aa, Artificial Sequence
  Synthetic peptide
QSSQAQ

SEQ ID NO: 574 - protein, 6 aa, Artificial Sequence
  Synthetic peptide
QSSQSQ

SEQ ID NO: 575 - protein, 6 aa, Artificial Sequence
  Synthetic peptide
QSSQSV

SEQ ID NO: 576 - protein, 6 aa, Artificial Sequence
  Synthetic peptide
QSSTSV

SEQ ID NO: 577 - protein, 6 aa, Artificial Sequence
  Synthetic peptide
QSSVSG

SEQ ID NO: 578 - protein, 6 aa, Artificial Sequence
  Synthetic peptide
QSSVSN

SEQ ID NO: 579 - protein, 6 aa, Artificial Sequence
  Synthetic peptide
QSSVSQ

SEQ ID NO: 580 - protein, 6 aa, Artificial Sequence
  Synthetic peptide
QSSVSS

SEQ ID NO: 581 - protein, 6 aa, Artificial Sequence
  Synthetic peptide
QSSVTG

SEQ ID NO: 582 - protein, 6 aa, Artificial Sequence
  Synthetic peptide
QSSVTS

SEQ ID NO: 583 - protein, 6 aa, Artificial Sequence
  Synthetic peptide
QTSQSQ

SEQ ID NO: 584 - protein, 6 aa, Artificial Sequence
  Synthetic peptide
QTSSSQ

SEQ ID NO: 585 - protein, 6 aa, Artificial Sequence
  Synthetic peptide
QTSSSV

SEQ ID NO: 586 - protein, 6 aa, Artificial Sequence
  Synthetic peptide
QTSVAG

SEQ ID NO: 587 - protein, 6 aa, Artificial Sequence
  Synthetic peptide
QTSVAS

SEQ ID NO: 588 - protein, 6 aa, Artificial Sequence
  Synthetic peptide
QTSVSG

SEQ ID NO: 589 - protein, 6 aa, Artificial Sequence
  Synthetic peptide
QTSVSN

SEQ ID NO: 590 - protein, 6 aa, Artificial Sequence
  Synthetic peptide
QTSVSS

SEQ ID NO: 591 - protein, 6 aa, Artificial Sequence
  Synthetic peptide
QTSVST

SEQ ID NO: 592 - protein, 6 aa, Artificial Sequence
  Synthetic peptide
QTSVTS

SEQ ID NO: 593 - protein, 6 aa, Artificial Sequence
  Synthetic peptide
QVSNSG

SEQ ID NO: 594 - protein, 6 aa, Artificial Sequence
  Synthetic peptide
QVSNSS

SEQ ID NO: 595 - protein, 6 aa, Artificial Sequence
  Synthetic peptide
QVSNST

SEQ ID NO: 596 - protein, 6 aa, Artificial Sequence
  Synthetic peptide
QVSQAQ

SEQ ID NO: 597 - protein, 6 aa, Artificial Sequence
  Synthetic peptide
QVSQTG

SEQ ID NO: 598 - protein, 6 aa, Artificial Sequence
  Synthetic peptide
QVSQTQ

SEQ ID NO: 599 - protein, 6 aa, Artificial Sequence
  Synthetic peptide
QVSSAQ

SEQ ID NO: 600 - protein, 6 aa, Artificial Sequence
  Synthetic peptide
QVSSSG

SEQ ID NO: 601 - protein, 6 aa, Artificial Sequence
  Synthetic peptide
QVSSSQ

SEQ ID NO: 602 - protein, 6 aa, Artificial Sequence
  Synthetic peptide
QVSSSS

SEQ ID NO: 603 - protein, 6 aa, Artificial Sequence
  Synthetic peptide
QVSSTQ

SEQ ID NO: 604 - protein, 6 aa, Artificial Sequence
  Synthetic peptide
QVSTSG

SEQ ID NO: 605 - protein, 6 aa, Artificial Sequence
  Synthetic peptide
QVSTSN

SEQ ID NO: 606 - protein, 6 aa, Artificial Sequence
  Synthetic peptide
QVSTSS

SEQ ID NO: 607 - protein, 6 aa, Artificial Sequence
  Synthetic peptide
QVSTST

SEQ ID NO: 608 - protein, 6 aa, Artificial Sequence
  Synthetic peptide
SAQQSQ

SEQ ID NO: 609 - protein, 6 aa, Artificial Sequence
  Synthetic peptide
SAQQTQ

SEQ ID NO: 610 - protein, 6 aa, Artificial Sequence
  Synthetic peptide
SASQQQ

SEQ ID NO: 611 - protein, 6 aa, Artificial Sequence
  Synthetic peptide
SASQSQ

SEQ ID NO: 612 - protein, 6 aa, Artificial Sequence
  Synthetic peptide
SGNSTV

SEQ ID NO: 613 - protein, 6 aa, Artificial Sequence
  Synthetic peptide
SGNTSV

SEQ ID NO: 614 - protein, 6 aa, Artificial Sequence
  Synthetic peptide
SGNVST

SEQ ID NO: 615 - protein, 6 aa, Artificial Sequence
  Synthetic peptide
SGNVTS

SEQ ID NO: 616 - protein, 6 aa, Artificial Sequence
  Synthetic peptide
SGQQTQ

SEQ ID NO: 617 - protein, 6 aa, Artificial Sequence
  Synthetic peptide
SGQQTV

SEQ ID NO: 618 - protein, 6 aa, Artificial Sequence
  Synthetic peptide
SGQTSV

SEQ ID NO: 619 - protein, 6 aa, Artificial Sequence
  Synthetic peptide
SGQVSS

SEQ ID NO: 620 - protein, 6 aa, Artificial Sequence
  Synthetic peptide
SGQVTG

SEQ ID NO: 621 - protein, 6 aa, Artificial Sequence
  Synthetic peptide
SGQVTQ

SEQ ID NO: 622 - protein, 6 aa, Artificial Sequence
  Synthetic peptide
SGQVTT

SEQ ID NO: 623 - protein, 6 aa, Artificial Sequence
  Synthetic peptide
SGSNTV

SEQ ID NO: 624 - protein, 6 aa, Artificial Sequence
  Synthetic peptide
SGSTNV

SEQ ID NO: 625 - protein, 6 aa, Artificial Sequence
  Synthetic peptide
SGSTQV

SEQ ID NO: 626 - protein, 6 aa, Artificial Sequence
  Synthetic peptide
SGSVAS

SEQ ID NO: 627 - protein, 6 aa, Artificial Sequence
  Synthetic peptide
SGSVNT

SEQ ID NO: 628 - protein, 6 aa, Artificial Sequence
  Synthetic peptide
SGSVQG

SEQ ID NO: 629 - protein, 6 aa, Artificial Sequence
  Synthetic peptide
SGSVQS

SEQ ID NO: 630 - protein, 6 aa, Artificial Sequence
  Synthetic peptide
SNNQTV

SEQ ID NO: 631 - protein, 6 aa, Artificial Sequence
  Synthetic peptide
SNNTAV

SEQ ID NO: 632 - protein, 6 aa, Artificial Sequence
  Synthetic peptide
SNNTNV

SEQ ID NO: 633 - protein, 6 aa, Artificial Sequence
  Synthetic peptide
SNNTQV

SEQ ID NO: 634 - protein, 6 aa, Artificial Sequence
  Synthetic peptide
SNQQSQ

SEQ ID NO: 635 - protein, 6 aa, Artificial Sequence
  Synthetic peptide
SNQQSV

SEQ ID NO: 636 - protein, 6 aa, Artificial Sequence
  Synthetic peptide
SNQQTQ

SEQ ID NO: 637 - protein, 6 aa, Artificial Sequence
  Synthetic peptide
SNQQTV

SEQ ID NO: 638 - protein, 6 aa, Artificial Sequence
  Synthetic peptide
SNQSTV

SEQ ID NO: 639 - protein, 6 aa, Artificial Sequence
  Synthetic peptide
SNQTSV

SEQ ID NO: 640 - protein, 6 aa, Artificial Sequence
  Synthetic peptide
SNQVSG

SEQ ID NO: 641 - protein, 6 aa, Artificial Sequence
  Synthetic peptide
SNQVSQ

SEQ ID NO: 642 - protein, 6 aa, Artificial Sequence
  Synthetic peptide
SNQVSS

SEQ ID NO: 643 - protein, 6 aa, Artificial Sequence
  Synthetic peptide
SNQVST

SEQ ID NO: 644 - protein, 6 aa, Artificial Sequence
  Synthetic peptide
SNQVTG

SEQ ID NO: 645 - protein, 6 aa, Artificial Sequence
  Synthetic peptide
SNQVTQ

SEQ ID NO: 646 - protein, 6 aa, Artificial Sequence
  Synthetic peptide
SNQVTS

SEQ ID NO: 647 - protein, 6 aa, Artificial Sequence
  Synthetic peptide
SNSGAV

SEQ ID NO: 648 - protein, 6 aa, Artificial Sequence
  Synthetic peptide
SNSNAV

SEQ ID NO: 649 - protein, 6 aa, Artificial Sequence
  Synthetic peptide
SNSNGV

SEQ ID NO: 650 - protein, 6 aa, Artificial Sequence
  Synthetic peptide
SNSNQS

SEQ ID NO: 651 - protein, 6 aa, Artificial Sequence
  Synthetic peptide
SNSQAG

SEQ ID NO: 652 - protein, 6 aa, Artificial Sequence
  Synthetic peptide
SNSQAQ

SEQ ID NO: 653 - protein, 6 aa, Artificial Sequence
  Synthetic peptide
SNSQAS

SEQ ID NO: 654 - protein, 6 aa, Artificial Sequence
  Synthetic peptide
SNSQAT

SEQ ID NO: 655 - protein, 6 aa, Artificial Sequence
  Synthetic peptide
SNSQAV

SEQ ID NO: 656 - protein, 6 aa, Artificial Sequence
  Synthetic peptide
SNSQGQ

SEQ ID NO: 657 - protein, 6 aa, Artificial Sequence
  Synthetic peptide
SNSQGV

SEQ ID NO: 658 - protein, 6 aa, Artificial Sequence
  Synthetic peptide
SNSQQA

SEQ ID NO: 659 - protein, 6 aa, Artificial Sequence
  Synthetic peptide
SNSQQG

SEQ ID NO: 660 - protein, 6 aa, Artificial Sequence
  Synthetic peptide
SNSQQQ

SEQ ID NO: 661 - protein, 6 aa, Artificial Sequence
  Synthetic peptide
SNSQQS

SEQ ID NO: 662 - protein, 6 aa, Artificial Sequence
  Synthetic peptide
SNSQQT

SEQ ID NO: 663 - protein, 6 aa, Artificial Sequence
  Synthetic peptide
SNSQQV

SEQ ID NO: 664 - protein, 6 aa, Artificial Sequence
  Synthetic peptide
SNSQSG

SEQ ID NO: 665 - protein, 6 aa, Artificial Sequence
  Synthetic peptide
SNSQST

SEQ ID NO: 666 - protein, 6 aa, Artificial Sequence
  Synthetic peptide
SNSQSV

SEQ ID NO: 667 - protein, 6 aa, Artificial Sequence
  Synthetic peptide
SNSSAV

SEQ ID NO: 668 - protein, 6 aa, Artificial Sequence
  Synthetic peptide
SNSSGV

SEQ ID NO: 669 - protein, 6 aa, Artificial Sequence
  Synthetic peptide
SNSSQG

SEQ ID NO: 670 - protein, 6 aa, Artificial Sequence
  Synthetic peptide
SNSSQQ

SEQ ID NO: 671 - protein, 6 aa, Artificial Sequence
  Synthetic peptide
SNSSQT

SEQ ID NO: 672 - protein, 6 aa, Artificial Sequence
  Synthetic peptide
SNSSQV

SEQ ID NO: 673 - protein, 6 aa, Artificial Sequence
  Synthetic peptide
SNSSSQ

SEQ ID NO: 674 - protein, 6 aa, Artificial Sequence
  Synthetic peptide
SNSSSV

SEQ ID NO: 675 - protein, 6 aa, Artificial Sequence
  Synthetic peptide
SNSTAG

SEQ ID NO: 676 - protein, 6 aa, Artificial Sequence
  Synthetic peptide
SNSTGS

SEQ ID NO: 677 - protein, 6 aa, Artificial Sequence
  Synthetic peptide
SNSTNV

SEQ ID NO: 678 - protein, 6 aa, Artificial Sequence
  Synthetic peptide
SNSTQA

SEQ ID NO: 679 - protein, 6 aa, Artificial Sequence
  Synthetic peptide
SNSTQQ

SEQ ID NO: 680 - protein, 6 aa, Artificial Sequence
  Synthetic peptide
SNSTQS

SEQ ID NO: 681 - protein, 6 aa, Artificial Sequence
  Synthetic peptide
SNSTQV

SEQ ID NO: 682 - protein, 6 aa, Artificial Sequence
  Synthetic peptide
SNSTSA

SEQ ID NO: 683 - protein, 6 aa, Artificial Sequence
  Synthetic peptide
SNSTSV

SEQ ID NO: 684 - protein, 6 aa, Artificial Sequence
  Synthetic peptide
SNSTVG

SEQ ID NO: 685 - protein, 6 aa, Artificial Sequence
  Synthetic peptide
SNSVAQ

SEQ ID NO: 686 - protein, 6 aa, Artificial Sequence
  Synthetic peptide
SNSVAS

SEQ ID NO: 687 - protein, 6 aa, Artificial Sequence
  Synthetic peptide
SNSVAT

SEQ ID NO: 688 - protein, 6 aa, Artificial Sequence
  Synthetic peptide
SNSVGQ

SEQ ID NO: 689 - protein, 6 aa, Artificial Sequence
  Synthetic peptide
SNSVGS

SEQ ID NO: 690 - protein, 6 aa, Artificial Sequence
  Synthetic peptide
SNSVGT

SEQ ID NO: 691 - protein, 6 aa, Artificial Sequence
  Synthetic peptide
SNSVNS

SEQ ID NO: 692 - protein, 6 aa, Artificial Sequence
  Synthetic peptide
SNSVNT

SEQ ID NO: 693 - protein, 6 aa, Artificial Sequence
  Synthetic peptide
SNSVQQ

SEQ ID NO: 694 - protein, 6 aa, Artificial Sequence
  Synthetic peptide
SNSVQS

SEQ ID NO: 695 - protein, 6 aa, Artificial Sequence
  Synthetic peptide
SNSVSG

SEQ ID NO: 696 - protein, 6 aa, Artificial Sequence
  Synthetic peptide
SNSVSQ

SEQ ID NO: 697 - protein, 6 aa, Artificial Sequence
  Synthetic peptide
SNSVSS

SEQ ID NO: 698 - protein, 6 aa, Artificial Sequence
  Synthetic peptide
SNSVST

SEQ ID NO: 699 - protein, 6 aa, Artificial Sequence
  Synthetic peptide
SNSVTG

SEQ ID NO: 700 - protein, 6 aa, Artificial Sequence
  Synthetic peptide
SQNVAS

SEQ ID NO: 701 - protein, 6 aa, Artificial Sequence
  Synthetic peptide
SQNVNS

SEQ ID NO: 702 - protein, 6 aa, Artificial Sequence
  Synthetic peptide
SQQQSQ

SEQ ID NO: 703 - protein, 6 aa, Artificial Sequence
  Synthetic peptide
SQQQSV

SEQ ID NO: 704 - protein, 6 aa, Artificial Sequence
  Synthetic peptide
SQQQTQ

SEQ ID NO: 705 - protein, 6 aa, Artificial Sequence
  Synthetic peptide
SQQQTV

SEQ ID NO: 706 - protein, 6 aa, Artificial Sequence
  Synthetic peptide
SQQSTV

SEQ ID NO: 707 - protein, 6 aa, Artificial Sequence
  Synthetic peptide
SQQVSG

SEQ ID NO: 708 - protein, 6 aa, Artificial Sequence
  Synthetic peptide
SQQVSN

SEQ ID NO: 709 - protein, 6 aa, Artificial Sequence
  Synthetic peptide
SQQVSS

SEQ ID NO: 710 - protein, 6 aa, Artificial Sequence
  Synthetic peptide
SQQVST

SEQ ID NO: 711 - protein, 6 aa, Artificial Sequence
  Synthetic peptide
SQQVTG

SEQ ID NO: 712 - protein, 6 aa, Artificial Sequence
  Synthetic peptide
SQQVTN

SEQ ID NO: 713 - protein, 6 aa, Artificial Sequence
  Synthetic peptide
SQQVTS

SEQ ID NO: 714 - protein, 6 aa, Artificial Sequence
  Synthetic peptide
SQQVTT

SEQ ID NO: 715 - protein, 6 aa, Artificial Sequence
  Synthetic peptide
SQSNAS

SEQ ID NO: 716 - protein, 6 aa, Artificial Sequence
  Synthetic peptide
SQSNAV

SEQ ID NO: 717 - protein, 6 aa, Artificial Sequence
  Synthetic peptide
SQSNGV

SEQ ID NO: 718 - protein, 6 aa, Artificial Sequence
  Synthetic peptide
SQSNQT

SEQ ID NO: 719 - protein, 6 aa, Artificial Sequence
  Synthetic peptide
SQSNQV

SEQ ID NO: 720 - protein, 6 aa, Artificial Sequence
  Synthetic peptide
SQSNSV

SEQ ID NO: 721 - protein, 6 aa, Artificial Sequence
  Synthetic peptide
SQSQAQ

SEQ ID NO: 722 - protein, 6 aa, Artificial Sequence
  Synthetic peptide
SQSQAS

SEQ ID NO: 723 - protein, 6 aa, Artificial Sequence
  Synthetic peptide
SQSQAV

SEQ ID NO: 724 - protein, 6 aa, Artificial Sequence
  Synthetic peptide
SQSQQQ

SEQ ID NO: 725 - protein, 6 aa, Artificial Sequence
  Synthetic peptide
SQSQQV

SEQ ID NO: 726 - protein, 6 aa, Artificial Sequence
  Synthetic peptide
SQSQSQ

SEQ ID NO: 727 - protein, 6 aa, Artificial Sequence
  Synthetic peptide
SQSSAV

SEQ ID NO: 728 - protein, 6 aa, Artificial Sequence
  Synthetic peptide
SQSSGV

SEQ ID NO: 729 - protein, 6 aa, Artificial Sequence
  Synthetic peptide
SQSSQG

SEQ ID NO: 730 - protein, 6 aa, Artificial Sequence
  Synthetic peptide
SQSSQV

SEQ ID NO: 731 - protein, 6 aa, Artificial Sequence
  Synthetic peptide
SQSSSQ

SEQ ID NO: 732 - protein, 6 aa, Artificial Sequence
  Synthetic peptide
SQSSSV

SEQ ID NO: 733 - protein, 6 aa, Artificial Sequence
  Synthetic peptide
SQSTAV

SEQ ID NO: 734 - protein, 6 aa, Artificial Sequence
  Synthetic peptide
SQSTQS

SEQ ID NO: 735 - protein, 6 aa, Artificial Sequence
  Synthetic peptide
SQSTSG

SEQ ID NO: 736 - protein, 6 aa, Artificial Sequence
  Synthetic peptide
SQSTSV

SEQ ID NO: 737 - protein, 6 aa, Artificial Sequence
  Synthetic peptide
SQSVAG

SEQ ID NO: 738 - protein, 6 aa, Artificial Sequence
  Synthetic peptide
SQSVAN

SEQ ID NO: 739 - protein, 6 aa, Artificial Sequence
  Synthetic peptide
SQSVAQ

SEQ ID NO: 740 - protein, 6 aa, Artificial Sequence
  Synthetic peptide
SQSVAS

SEQ ID NO: 741 - protein, 6 aa, Artificial Sequence
  Synthetic peptide
SQSVAT

SEQ ID NO: 742 - protein, 6 aa, Artificial Sequence
  Synthetic peptide
SQSVGG

SEQ ID NO: 743 - protein, 6 aa, Artificial Sequence
  Synthetic peptide
SQSVGN

SEQ ID NO: 744 - protein, 6 aa, Artificial Sequence
  Synthetic peptide
SQSVGQ

SEQ ID NO: 745 - protein, 6 aa, Artificial Sequence
  Synthetic peptide
SQSVGS

SEQ ID NO: 746 - protein, 6 aa, Artificial Sequence
  Synthetic peptide
SQSVGT

SEQ ID NO: 747 - protein, 6 aa, Artificial Sequence
  Synthetic peptide
SQSVNG

SEQ ID NO: 748 - protein, 6 aa, Artificial Sequence
  Synthetic peptide
SQSVNS

SEQ ID NO: 749 - protein, 6 aa, Artificial Sequence
  Synthetic peptide
SQSVQG

SEQ ID NO: 750 - protein, 6 aa, Artificial Sequence
  Synthetic peptide
SQSVQN

SEQ ID NO: 751 - protein, 6 aa, Artificial Sequence
  Synthetic peptide
SQSVQQ

SEQ ID NO: 752 - protein, 6 aa, Artificial Sequence
  Synthetic peptide
SQSVQS

SEQ ID NO: 753 - protein, 6 aa, Artificial Sequence
  Synthetic peptide
SQSVQT

SEQ ID NO: 754 - protein, 6 aa, Artificial Sequence
  Synthetic peptide
SQSVSG

SEQ ID NO: 755 - protein, 6 aa, Artificial Sequence
  Synthetic peptide
SQSVSN

SEQ ID NO: 756 - protein, 6 aa, Artificial Sequence
  Synthetic peptide
SQSVSS

SEQ ID NO: 757 - protein, 6 aa, Artificial Sequence
  Synthetic peptide
SQSVST

SEQ ID NO: 758 - protein, 6 aa, Artificial Sequence
  Synthetic peptide
SSNGTV

SEQ ID NO: 759 - protein, 6 aa, Artificial Sequence
  Synthetic peptide
SSNQNQ

SEQ ID NO: 760 - protein, 6 aa, Artificial Sequence
  Synthetic peptide
SSNQNV

SEQ ID NO: 761 - protein, 6 aa, Artificial Sequence
  Synthetic peptide
SSNQTQ

SEQ ID NO: 762 - protein, 6 aa, Artificial Sequence
  Synthetic peptide
SSNQTV

SEQ ID NO: 763 - protein, 6 aa, Artificial Sequence
  Synthetic peptide
SSNSTV

SEQ ID NO: 764 - protein, 6 aa, Artificial Sequence
  Synthetic peptide
SSNTNV

SEQ ID NO: 765 - protein, 6 aa, Artificial Sequence
  Synthetic peptide
SSNTQV

SEQ ID NO: 766 - protein, 6 aa, Artificial Sequence
  Synthetic peptide
SSNTVG

SEQ ID NO: 767 - protein, 6 aa, Artificial Sequence
  Synthetic peptide
SSNVAQ

SEQ ID NO: 768 - protein, 6 aa, Artificial Sequence
  Synthetic peptide
SSNVAS

SEQ ID NO: 769 - protein, 6 aa, Artificial Sequence
  Synthetic peptide
SSNVGT

SEQ ID NO: 770 - protein, 6 aa, Artificial Sequence
  Synthetic peptide
SSNVNG

SEQ ID NO: 771 - protein, 6 aa, Artificial Sequence
  Synthetic peptide
SSNVNQ

SEQ ID NO: 772 - protein, 6 aa, Artificial Sequence
  Synthetic peptide
SSNVNS

SEQ ID NO: 773 - protein, 6 aa, Artificial Sequence
  Synthetic peptide
SSNVNT

SEQ ID NO: 774 - protein, 6 aa, Artificial Sequence
  Synthetic peptide
SSNVQS

SEQ ID NO: 775 - protein, 6 aa, Artificial Sequence
  Synthetic peptide
SSNVQT

SEQ ID NO: 776 - protein, 6 aa, Artificial Sequence
  Synthetic peptide
SSNVTG

SEQ ID NO: 777 - protein, 6 aa, Artificial Sequence
  Synthetic peptide
SSNVTQ

SEQ ID NO: 778 - protein, 6 aa, Artificial Sequence
  Synthetic peptide
SSNVTS

SEQ ID NO: 779 - protein, 6 aa, Artificial Sequence
  Synthetic peptide
SSQNTV

SEQ ID NO: 780 - protein, 6 aa, Artificial Sequence
  Synthetic peptide
SSQQSV

SEQ ID NO: 781 - protein, 6 aa, Artificial Sequence
  Synthetic peptide
SSQQTQ

SEQ ID NO: 782 - protein, 6 aa, Artificial Sequence
  Synthetic peptide
SSQQTV

SEQ ID NO: 783 - protein, 6 aa, Artificial Sequence
  Synthetic peptide
SSQSTQ

SEQ ID NO: 784 - protein, 6 aa, Artificial Sequence
  Synthetic peptide
SSQTSV

SEQ ID NO: 785 - protein, 6 aa, Artificial Sequence
  Synthetic peptide
SSQVNS

SEQ ID NO: 786 - protein, 6 aa, Artificial Sequence
  Synthetic peptide
SSQVSN

SEQ ID NO: 787 - protein, 6 aa, Artificial Sequence
  Synthetic peptide
SSQVSQ

SEQ ID NO: 788 - protein, 6 aa, Artificial Sequence
  Synthetic peptide
SSQVSS

SEQ ID NO: 789 - protein, 6 aa, Artificial Sequence
  Synthetic peptide
SSQVST

SEQ ID NO: 790 - protein, 6 aa, Artificial Sequence
  Synthetic peptide
SSQVTG

SEQ ID NO: 791 - protein, 6 aa, Artificial Sequence
  Synthetic peptide
SSQVTQ

SEQ ID NO: 792 - protein, 6 aa, Artificial Sequence
  Synthetic peptide
SSQVTS

SEQ ID NO: 793 - protein, 6 aa, Artificial Sequence
  Synthetic peptide
SSQVTT

SEQ ID NO: 794 - protein, 6 aa, Artificial Sequence
  Synthetic peptide
SSSNAV

SEQ ID NO: 795 - protein, 6 aa, Artificial Sequence
  Synthetic peptide
SSSNQV

SEQ ID NO: 796 - protein, 6 aa, Artificial Sequence
  Synthetic peptide
SSSNSV

SEQ ID NO: 797 - protein, 6 aa, Artificial Sequence
  Synthetic peptide
SSSQAQ

SEQ ID NO: 798 - protein, 6 aa, Artificial Sequence
  Synthetic peptide
SSSQNQ

SEQ ID NO: 799 - protein, 6 aa, Artificial Sequence
  Synthetic peptide
SSSQQQ

SEQ ID NO: 800 - protein, 6 aa, Artificial Sequence
  Synthetic peptide
SSSQQT

SEQ ID NO: 801 - protein, 6 aa, Artificial Sequence
  Synthetic peptide
SSSQQV

SEQ ID NO: 802 - protein, 6 aa, Artificial Sequence
  Synthetic peptide
SSSQSQ

SEQ ID NO: 803 - protein, 6 aa, Artificial Sequence
  Synthetic peptide
SSSQSV

SEQ ID NO: 804 - protein, 6 aa, Artificial Sequence
  Synthetic peptide
SSSSGQ

SEQ ID NO: 805 - protein, 6 aa, Artificial Sequence
  Synthetic peptide
SSSSGV

SEQ ID NO: 806 - protein, 6 aa, Artificial Sequence
  Synthetic peptide
SSSSNV

SEQ ID NO: 807 - protein, 6 aa, Artificial Sequence
  Synthetic peptide
SSSSQQ

SEQ ID NO: 808 - protein, 6 aa, Artificial Sequence
  Synthetic peptide
SSSSQS

SEQ ID NO: 809 - protein, 6 aa, Artificial Sequence
  Synthetic peptide
SSSTAV

SEQ ID NO: 810 - protein, 6 aa, Artificial Sequence
  Synthetic peptide
SSSTNV

SEQ ID NO: 811 - protein, 6 aa, Artificial Sequence
  Synthetic peptide
SSSTSV

SEQ ID NO: 812 - protein, 6 aa, Artificial Sequence
  Synthetic peptide
SSSVAQ

SEQ ID NO: 813 - protein, 6 aa, Artificial Sequence
  Synthetic peptide
SSSVAS

SEQ ID NO: 814 - protein, 6 aa, Artificial Sequence
  Synthetic peptide
SSSVGG

SEQ ID NO: 815 - protein, 6 aa, Artificial Sequence
  Synthetic peptide
SSSVGQ

SEQ ID NO: 816 - protein, 6 aa, Artificial Sequence
  Synthetic peptide
SSSVGS

SEQ ID NO: 817 - protein, 6 aa, Artificial Sequence
  Synthetic peptide
SSSVNG

SEQ ID NO: 818 - protein, 6 aa, Artificial Sequence
  Synthetic peptide
SSSVNS

SEQ ID NO: 819 - protein, 6 aa, Artificial Sequence
  Synthetic peptide
SSSVNT

SEQ ID NO: 820 - protein, 6 aa, Artificial Sequence
  Synthetic peptide
SSSVQG

SEQ ID NO: 821 - protein, 6 aa, Artificial Sequence
  Synthetic peptide
SSSVQN

SEQ ID NO: 822 - protein, 6 aa, Artificial Sequence
  Synthetic peptide
SSSVQS

SEQ ID NO: 823 - protein, 6 aa, Artificial Sequence
  Synthetic peptide
SSSVQT

SEQ ID NO: 824 - protein, 6 aa, Artificial Sequence
  Synthetic peptide
SSSVSG

SEQ ID NO: 825 - protein, 6 aa, Artificial Sequence
  Synthetic peptide
SSSVSN

SEQ ID NO: 826 - protein, 6 aa, Artificial Sequence
  Synthetic peptide
SSSVSQ

SEQ ID NO: 827 - protein, 6 aa, Artificial Sequence
  Synthetic peptide
SSSVSS

SEQ ID NO: 828 - protein, 6 aa, Artificial Sequence
  Synthetic peptide
SSSVST

SEQ ID NO: 829 - protein, 6 aa, Artificial Sequence
  Synthetic peptide
STNGSV

SEQ ID NO: 830 - protein, 6 aa, Artificial Sequence
  Synthetic peptide
STNSGV

SEQ ID NO: 831 - protein, 6 aa, Artificial Sequence
  Synthetic peptide
STNSNV

SEQ ID NO: 832 - protein, 6 aa, Artificial Sequence
  Synthetic peptide
STNTNV

SEQ ID NO: 833 - protein, 6 aa, Artificial Sequence
  Synthetic peptide
STNTQV

SEQ ID NO: 834 - protein, 6 aa, Artificial Sequence
  Synthetic peptide
STNVGS

SEQ ID NO: 835 - protein, 6 aa, Artificial Sequence
  Synthetic peptide
STNVNG

SEQ ID NO: 836 - protein, 6 aa, Artificial Sequence
  Synthetic peptide
STNVNS

SEQ ID NO: 837 - protein, 6 aa, Artificial Sequence
  Synthetic peptide
STNVSG

SEQ ID NO: 838 - protein, 6 aa, Artificial Sequence
  Synthetic peptide
STNVSS

SEQ ID NO: 839 - protein, 6 aa, Artificial Sequence
  Synthetic peptide
STNVTG

SEQ ID NO: 840 - protein, 6 aa, Artificial Sequence
  Synthetic peptide
STNVTS

SEQ ID NO: 841 - protein, 6 aa, Artificial Sequence
  Synthetic peptide
STQQSG

SEQ ID NO: 842 - protein, 6 aa, Artificial Sequence
  Synthetic peptide
STQQSQ

SEQ ID NO: 843 - protein, 6 aa, Artificial Sequence
  Synthetic peptide
STQQSV

SEQ ID NO: 844 - protein, 6 aa, Artificial Sequence
  Synthetic peptide
STQQTQ

SEQ ID NO: 845 - protein, 6 aa, Artificial Sequence
  Synthetic peptide
STQQTS

SEQ ID NO: 846 - protein, 6 aa, Artificial Sequence
  Synthetic peptide
STQQTV

SEQ ID NO: 847 - protein, 6 aa, Artificial Sequence
  Synthetic peptide
STQSNV

SEQ ID NO: 848 - protein, 6 aa, Artificial Sequence
  Synthetic peptide
STQSTV

SEQ ID NO: 849 - protein, 6 aa, Artificial Sequence
  Synthetic peptide
STQTSS

SEQ ID NO: 850 - protein, 6 aa, Artificial Sequence
  Synthetic peptide
STQTSV

SEQ ID NO: 851 - protein, 6 aa, Artificial Sequence
  Synthetic peptide
STQVNS

SEQ ID NO: 852 - protein, 6 aa, Artificial Sequence
  Synthetic peptide
STQVSG

SEQ ID NO: 853 - protein, 6 aa, Artificial Sequence
  Synthetic peptide
STQVSN

SEQ ID NO: 854 - protein, 6 aa, Artificial Sequence
  Synthetic peptide
STQVST

SEQ ID NO: 855 - protein, 6 aa, Artificial Sequence
  Synthetic peptide
STQVTG

SEQ ID NO: 856 - protein, 6 aa, Artificial Sequence
  Synthetic peptide
STQVTN

SEQ ID NO: 857 - protein, 6 aa, Artificial Sequence
  Synthetic peptide
STQVTQ

SEQ ID NO: 858 - protein, 6 aa, Artificial Sequence
  Synthetic peptide
STQVTS

SEQ ID NO: 859 - protein, 6 aa, Artificial Sequence
  Synthetic peptide
STQVTT

SEQ ID NO: 860 - protein, 6 aa, Artificial Sequence
  Synthetic peptide
STSGNV

SEQ ID NO: 861 - protein, 6 aa, Artificial Sequence
  Synthetic peptide
STSNAS

SEQ ID NO: 862 - protein, 6 aa, Artificial Sequence
  Synthetic peptide
STSNGV

SEQ ID NO: 863 - protein, 6 aa, Artificial Sequence
  Synthetic peptide
STSNQV

SEQ ID NO: 864 - protein, 6 aa, Artificial Sequence
  Synthetic peptide
STSNSV

SEQ ID NO: 865 - protein, 6 aa, Artificial Sequence
  Synthetic peptide
STSQAQ

SEQ ID NO: 866 - protein, 6 aa, Artificial Sequence
  Synthetic peptide
STSQGV

SEQ ID NO: 867 - protein, 6 aa, Artificial Sequence
  Synthetic peptide
STSQNQ

SEQ ID NO: 868 - protein, 6 aa, Artificial Sequence
  Synthetic peptide
STSQQQ

SEQ ID NO: 869 - protein, 6 aa, Artificial Sequence
  Synthetic peptide
STSQQV

SEQ ID NO: 870 - protein, 6 aa, Artificial Sequence
  Synthetic peptide
STSQSQ

SEQ ID NO: 871 - protein, 6 aa, Artificial Sequence
  Synthetic peptide
STSSAV

SEQ ID NO: 872 - protein, 6 aa, Artificial Sequence
  Synthetic peptide
STSSGV

SEQ ID NO: 873 - protein, 6 aa, Artificial Sequence
  Synthetic peptide
STSSQQ

SEQ ID NO: 874 - protein, 6 aa, Artificial Sequence
  Synthetic peptide
STSSQS

SEQ ID NO: 875 - protein, 6 aa, Artificial Sequence
  Synthetic peptide
STSSQV

SEQ ID NO: 876 - protein, 6 aa, Artificial Sequence
  Synthetic peptide
STSSSQ

SEQ ID NO: 877 - protein, 6 aa, Artificial Sequence
  Synthetic peptide
STSTAV

SEQ ID NO: 878 - protein, 6 aa, Artificial Sequence
  Synthetic peptide
STSTGV

SEQ ID NO: 879 - protein, 6 aa, Artificial Sequence
  Synthetic peptide
STSTQA

SEQ ID NO: 880 - protein, 6 aa, Artificial Sequence
  Synthetic peptide
STSTQG

SEQ ID NO: 881 - protein, 6 aa, Artificial Sequence
  Synthetic peptide
STSTQT

SEQ ID NO: 882 - protein, 6 aa, Artificial Sequence
  Synthetic peptide
STSTQV

SEQ ID NO: 883 - protein, 6 aa, Artificial Sequence
  Synthetic peptide
STSTSQ

SEQ ID NO: 884 - protein, 6 aa, Artificial Sequence
  Synthetic peptide
STSTSV

SEQ ID NO: 885 - protein, 6 aa, Artificial Sequence
  Synthetic peptide
STSVAG

SEQ ID NO: 886 - protein, 6 aa, Artificial Sequence
  Synthetic peptide
STSVAN

SEQ ID NO: 887 - protein, 6 aa, Artificial Sequence
  Synthetic peptide
STSVAQ

SEQ ID NO: 888 - protein, 6 aa, Artificial Sequence
  Synthetic peptide
STSVAS

SEQ ID NO: 889 - protein, 6 aa, Artificial Sequence
  Synthetic peptide
STSVAT

SEQ ID NO: 890 - protein, 6 aa, Artificial Sequence
  Synthetic peptide
STSVGG

SEQ ID NO: 891 - protein, 6 aa, Artificial Sequence
  Synthetic peptide
STSVGN

SEQ ID NO: 892 - protein, 6 aa, Artificial Sequence
  Synthetic peptide
STSVGQ

SEQ ID NO: 893 - protein, 6 aa, Artificial Sequence
  Synthetic peptide
STSVGS

SEQ ID NO: 894 - protein, 6 aa, Artificial Sequence
  Synthetic peptide
STSVNG

SEQ ID NO: 895 - protein, 6 aa, Artificial Sequence
  Synthetic peptide
STSVNN

SEQ ID NO: 896 - protein, 6 aa, Artificial Sequence
  Synthetic peptide
STSVNS

SEQ ID NO: 897 - protein, 6 aa, Artificial Sequence
  Synthetic peptide
STSVQG

SEQ ID NO: 898 - protein, 6 aa, Artificial Sequence
  Synthetic peptide
STSVQN

SEQ ID NO: 899 - protein, 6 aa, Artificial Sequence
  Synthetic peptide
STSVQQ

SEQ ID NO: 900 - protein, 6 aa, Artificial Sequence
  Synthetic peptide
STSVQS

SEQ ID NO: 901 - protein, 6 aa, Artificial Sequence
  Synthetic peptide
STSVQT

SEQ ID NO: 902 - protein, 6 aa, Artificial Sequence
  Synthetic peptide
STSVSG

SEQ ID NO: 903 - protein, 6 aa, Artificial Sequence
  Synthetic peptide
STSVSN

SEQ ID NO: 904 - protein, 6 aa, Artificial Sequence
  Synthetic peptide
STSVSQ

SEQ ID NO: 905 - protein, 6 aa, Artificial Sequence
  Synthetic peptide
STSVSS

SEQ ID NO: 906 - protein, 6 aa, Artificial Sequence
  Synthetic peptide
STSVST

SEQ ID NO: 907 - protein, 6 aa, Artificial Sequence
  Synthetic peptide
SVNGST

SEQ ID NO: 908 - protein, 6 aa, Artificial Sequence
  Synthetic peptide
SVNGTS

SEQ ID NO: 909 - protein, 6 aa, Artificial Sequence
  Synthetic peptide
SVNQAQ

SEQ ID NO: 910 - protein, 6 aa, Artificial Sequence
  Synthetic peptide
SVNQQQ

SEQ ID NO: 911 - protein, 6 aa, Artificial Sequence
  Synthetic peptide
SVNSGT

SEQ ID NO: 912 - protein, 6 aa, Artificial Sequence
  Synthetic peptide
SVNSNQ

SEQ ID NO: 913 - protein, 6 aa, Artificial Sequence
  Synthetic peptide
SVNSNS

SEQ ID NO: 914 - protein, 6 aa, Artificial Sequence
  Synthetic peptide
SVNSTG

SEQ ID NO: 915 - protein, 6 aa, Artificial Sequence
  Synthetic peptide
SVNSTS

SEQ ID NO: 916 - protein, 6 aa, Artificial Sequence
  Synthetic peptide
SVNTGS

SEQ ID NO: 917 - protein, 6 aa, Artificial Sequence
  Synthetic peptide
SVNTSG

SEQ ID NO: 918 - protein, 6 aa, Artificial Sequence
  Synthetic peptide
SVQQSQ

SEQ ID NO: 919 - protein, 6 aa, Artificial Sequence
  Synthetic peptide
SVQQST

SEQ ID NO: 920 - protein, 6 aa, Artificial Sequence
  Synthetic peptide
SVQQTQ

SEQ ID NO: 921 - protein, 6 aa, Artificial Sequence
  Synthetic peptide
SVQQTS

SEQ ID NO: 922 - protein, 6 aa, Artificial Sequence
  Synthetic peptide
SVQQTT

SEQ ID NO: 923 - protein, 6 aa, Artificial Sequence
  Synthetic peptide
SVQSNQ

SEQ ID NO: 924 - protein, 6 aa, Artificial Sequence
  Synthetic peptide
SVQSNS

SEQ ID NO: 925 - protein, 6 aa, Artificial Sequence
  Synthetic peptide
SVQSSG

SEQ ID NO: 926 - protein, 6 aa, Artificial Sequence
  Synthetic peptide
SVQSSQ

SEQ ID NO: 927 - protein, 6 aa, Artificial Sequence
  Synthetic peptide
SVQSSS

SEQ ID NO: 928 - protein, 6 aa, Artificial Sequence
  Synthetic peptide
SVQSTG

SEQ ID NO: 929 - protein, 6 aa, Artificial Sequence
  Synthetic peptide
SVQSTQ

SEQ ID NO: 930 - protein, 6 aa, Artificial Sequence
  Synthetic peptide
SVQSTS

SEQ ID NO: 931 - protein, 6 aa, Artificial Sequence
  Synthetic peptide
SVQTSG

SEQ ID NO: 932 - protein, 6 aa, Artificial Sequence
  Synthetic peptide
SVQTSN

SEQ ID NO: 933 - protein, 6 aa, Artificial Sequence
  Synthetic peptide
SVQTSS

SEQ ID NO: 934 - protein, 6 aa, Artificial Sequence
  Synthetic peptide
SVQVSN

SEQ ID NO: 935 - protein, 6 aa, Artificial Sequence
  Synthetic peptide
SVSGNT

SEQ ID NO: 936 - protein, 6 aa, Artificial Sequence
  Synthetic peptide
SVSGQS

SEQ ID NO: 937 - protein, 6 aa, Artificial Sequence
  Synthetic peptide
SVSNAQ

SEQ ID NO: 938 - protein, 6 aa, Artificial Sequence
  Synthetic peptide
SVSNAS

SEQ ID NO: 939 - protein, 6 aa, Artificial Sequence
  Synthetic peptide
SVSNGS

SEQ ID NO: 940 - protein, 6 aa, Artificial Sequence
  Synthetic peptide
SVSNGT

SEQ ID NO: 941 - protein, 6 aa, Artificial Sequence
  Synthetic peptide
SVSNQG

SEQ ID NO: 942 - protein, 6 aa, Artificial Sequence
  Synthetic peptide
SVSNQS

SEQ ID NO: 943 - protein, 6 aa, Artificial Sequence
  Synthetic peptide
SVSNQT

SEQ ID NO: 944 - protein, 6 aa, Artificial Sequence
  Synthetic peptide
SVSNST

SEQ ID NO: 945 - protein, 6 aa, Artificial Sequence
  Synthetic peptide
SVSNTG

SEQ ID NO: 946 - protein, 6 aa, Artificial Sequence
  Synthetic peptide
SVSQAQ

SEQ ID NO: 947 - protein, 6 aa, Artificial Sequence
  Synthetic peptide
SVSQAS

SEQ ID NO: 948 - protein, 6 aa, Artificial Sequence
  Synthetic peptide
SVSQGQ

SEQ ID NO: 949 - protein, 6 aa, Artificial Sequence
  Synthetic peptide
SVSQNQ

SEQ ID NO: 950 - protein, 6 aa, Artificial Sequence
  Synthetic peptide
SVSQQG

SEQ ID NO: 951 - protein, 6 aa, Artificial Sequence
  Synthetic peptide
SVSQQQ

SEQ ID NO: 952 - protein, 6 aa, Artificial Sequence
  Synthetic peptide
SVSQQS

SEQ ID NO: 953 - protein, 6 aa, Artificial Sequence
  Synthetic peptide
SVSQSG

SEQ ID NO: 954 - protein, 6 aa, Artificial Sequence
  Synthetic peptide
SVSQSS

SEQ ID NO: 955 - protein, 6 aa, Artificial Sequence
  Synthetic peptide
SVSQTQ

SEQ ID NO: 956 - protein, 6 aa, Artificial Sequence
  Synthetic peptide
SVSSAQ

SEQ ID NO: 957 - protein, 6 aa, Artificial Sequence
  Synthetic peptide
SVSSAS

SEQ ID NO: 958 - protein, 6 aa, Artificial Sequence
  Synthetic peptide
SVSSGG

SEQ ID NO: 959 - protein, 6 aa, Artificial Sequence
  Synthetic peptide
SVSSGS

SEQ ID NO: 960 - protein, 6 aa, Artificial Sequence
  Synthetic peptide
SVSSGT

SEQ ID NO: 961 - protein, 6 aa, Artificial Sequence
  Synthetic peptide
SVSSNG

SEQ ID NO: 962 - protein, 6 aa, Artificial Sequence
  Synthetic peptide
SVSSNQ

SEQ ID NO: 963 - protein, 6 aa, Artificial Sequence
  Synthetic peptide
SVSSNS

SEQ ID NO: 964 - protein, 6 aa, Artificial Sequence
  Synthetic peptide
SVSSNT

SEQ ID NO: 965 - protein, 6 aa, Artificial Sequence
  Synthetic peptide
SVSSQG

SEQ ID NO: 966 - protein, 6 aa, Artificial Sequence
  Synthetic peptide
SVSSQQ

SEQ ID NO: 967 - protein, 6 aa, Artificial Sequence
  Synthetic peptide
SVSSQS

SEQ ID NO: 968 - protein, 6 aa, Artificial Sequence
  Synthetic peptide
SVSSQT

SEQ ID NO: 969 - protein, 6 aa, Artificial Sequence
  Synthetic peptide
SVSSQV

SEQ ID NO: 970 - protein, 6 aa, Artificial Sequence
  Synthetic peptide
SVSSSG

SEQ ID NO: 971 - protein, 6 aa, Artificial Sequence
  Synthetic peptide
SVSSSQ

SEQ ID NO: 972 - protein, 6 aa, Artificial Sequence
  Synthetic peptide
SVSSSS

SEQ ID NO: 973 - protein, 6 aa, Artificial Sequence
  Synthetic peptide
SVSTAG

SEQ ID NO: 974 - protein, 6 aa, Artificial Sequence
  Synthetic peptide
SVSTAS

SEQ ID NO: 975 - protein, 6 aa, Artificial Sequence
  Synthetic peptide
SVSTAT

SEQ ID NO: 976 - protein, 6 aa, Artificial Sequence
  Synthetic peptide
SVSTGS

SEQ ID NO: 977 - protein, 6 aa, Artificial Sequence
  Synthetic peptide
SVSTNS

SEQ ID NO: 978 - protein, 6 aa, Artificial Sequence
  Synthetic peptide
SVSTNT

SEQ ID NO: 979 - protein, 6 aa, Artificial Sequence
  Synthetic peptide
SVSTQQ

SEQ ID NO: 980 - protein, 6 aa, Artificial Sequence
  Synthetic peptide
SVSTQS

SEQ ID NO: 981 - protein, 6 aa, Artificial Sequence
  Synthetic peptide
SVSTQT

SEQ ID NO: 982 - protein, 6 aa, Artificial Sequence
  Synthetic peptide
SVSTSG

SEQ ID NO: 983 - protein, 6 aa, Artificial Sequence
  Synthetic peptide
SVSTSN

SEQ ID NO: 984 - protein, 6 aa, Artificial Sequence
  Synthetic peptide
SVSTSS

SEQ ID NO: 985 - protein, 6 aa, Artificial Sequence
  Synthetic peptide
SVSTST

SEQ ID NO: 986 - protein, 6 aa, Artificial Sequence
  Synthetic peptide
TASQSQ

SEQ ID NO: 987 - protein, 6 aa, Artificial Sequence
  Synthetic peptide
TASQVQ

SEQ ID NO: 988 - protein, 6 aa, Artificial Sequence
  Synthetic peptide
TGSNSV

SEQ ID NO: 989 - protein, 6 aa, Artificial Sequence
  Synthetic peptide
TGSNVS

SEQ ID NO: 990 - protein, 6 aa, Artificial Sequence
  Synthetic peptide
TGSSNV

SEQ ID NO: 991 - protein, 6 aa, Artificial Sequence
  Synthetic peptide
TGSVNS

SEQ ID NO: 992 - protein, 6 aa, Artificial Sequence
  Synthetic peptide
TGSVSN

SEQ ID NO: 993 - protein, 6 aa, Artificial Sequence
  Synthetic peptide
TNSGSV

SEQ ID NO: 994 - protein, 6 aa, Artificial Sequence
  Synthetic peptide
TNSGVS

SEQ ID NO: 995 - protein, 6 aa, Artificial Sequence
  Synthetic peptide
TNSQVQ

SEQ ID NO: 996 - protein, 6 aa, Artificial Sequence
  Synthetic peptide
TNSSGV

SEQ ID NO: 997 - protein, 6 aa, Artificial Sequence
  Synthetic peptide
TNSVGS

SEQ ID NO: 998 - protein, 6 aa, Artificial Sequence
  Synthetic peptide
TNSVSG

SEQ ID NO: 999 - protein, 6 aa, Artificial Sequence
  Synthetic peptide
TQSQSQ

SEQ ID NO: 1000 - protein, 6 aa, Artificial Sequence
  Synthetic peptide
TQSQVQ

SEQ ID NO: 1001 - protein, 6 aa, Artificial Sequence
  Synthetic peptide
TQSTVS

SEQ ID NO: 1002 - protein, 6 aa, Artificial Sequence
  Synthetic peptide
TQSVSN

SEQ ID NO: 1003 - protein, 6 aa, Artificial Sequence
  Synthetic peptide
TQSVSS

SEQ ID NO: 1004 - protein, 6 aa, Artificial Sequence
  Synthetic peptide
TSNGVS

SEQ ID NO: 1005 - protein, 6 aa, Artificial Sequence
  Synthetic peptide
TSNVGS

SEQ ID NO: 1006 - protein, 6 aa, Artificial Sequence
  Synthetic peptide
TSNVSG

SEQ ID NO: 1007 - protein, 6 aa, Artificial Sequence
  Synthetic peptide
TSSGNV

SEQ ID NO: 1008 - protein, 6 aa, Artificial Sequence
  Synthetic peptide
TSSGVN

SEQ ID NO: 1009 - protein, 6 aa, Artificial Sequence
  Synthetic peptide
TSSNGV

SEQ ID NO: 1010 - protein, 6 aa, Artificial Sequence
  Synthetic peptide
TSSNSV

SEQ ID NO: 1011 - protein, 6 aa, Artificial Sequence
  Synthetic peptide
TSSNVG

SEQ ID NO: 1012 - protein, 6 aa, Artificial Sequence
  Synthetic peptide
TSSQSQ

SEQ ID NO: 1013 - protein, 6 aa, Artificial Sequence
  Synthetic peptide
TSSSVQ

SEQ ID NO: 1014 - protein, 6 aa, Artificial Sequence
  Synthetic peptide
TSSVGN

SEQ ID NO: 1015 - protein, 6 aa, Artificial Sequence
  Synthetic peptide
TSSVNG

SEQ ID NO: 1016 - protein, 6 aa, Artificial Sequence
  Synthetic peptide
TSSVSG

SEQ ID NO: 1017 - protein, 6 aa, Artificial Sequence
  Synthetic peptide
TSSVSS

SEQ ID NO: 1018 - protein, 6 aa, Artificial Sequence
  Synthetic peptide
TSSVTS

SEQ ID NO: 1019 - protein, 6 aa, Artificial Sequence
  Synthetic peptide
TVNSGS

SEQ ID NO: 1020 - protein, 6 aa, Artificial Sequence
  Synthetic peptide
TVNSSG

SEQ ID NO: 1021 - protein, 6 aa, Artificial Sequence
  Synthetic peptide
TVSGNS

SEQ ID NO: 1022 - protein, 6 aa, Artificial Sequence
  Synthetic peptide
TVSGSN

SEQ ID NO: 1023 - protein, 6 aa, Artificial Sequence
  Synthetic peptide
TVSNGS

SEQ ID NO: 1024 - protein, 6 aa, Artificial Sequence
  Synthetic peptide
TVSNSG

SEQ ID NO: 1025 - protein, 6 aa, Artificial Sequence
  Synthetic peptide
TVSNST

SEQ ID NO: 1026 - protein, 6 aa, Artificial Sequence
  Synthetic peptide
TVSNVG

SEQ ID NO: 1027 - protein, 6 aa, Artificial Sequence
  Synthetic peptide
TVSQNQ

SEQ ID NO: 1028 - protein, 6 aa, Artificial Sequence
  Synthetic peptide
TVSQQV

SEQ ID NO: 1029 - protein, 6 aa, Artificial Sequence
  Synthetic peptide
TVSQSG

SEQ ID NO: 1030 - protein, 6 aa, Artificial Sequence
  Synthetic peptide
TVSQSQ

SEQ ID NO: 1031 - protein, 6 aa, Artificial Sequence
  Synthetic peptide
TVSSGN

SEQ ID NO: 1032 - protein, 6 aa, Artificial Sequence
  Synthetic peptide
TVSSNG

SEQ ID NO: 1033 - protein, 6 aa, Artificial Sequence
  Synthetic peptide
TVSSNQ

SEQ ID NO: 1034 - protein, 6 aa, Artificial Sequence
  Synthetic peptide
TVSSNS

SEQ ID NO: 1035 - protein, 6 aa, Artificial Sequence
  Synthetic peptide
TVSSSQ

SEQ ID NO: 1036 - protein, 6 aa, Artificial Sequence
  Synthetic peptide
TVSTSG

SEQ ID NO: 1037 - protein, 6 aa, Artificial Sequence
  Synthetic peptide
TVSTSN

SEQ ID NO: 1038 - protein, 6 aa, Artificial Sequence
  Synthetic peptide
TVSTSS

SEQ ID NO: 1039 - protein, 6 aa, Artificial Sequence
  Synthetic peptide
TVSTST

SEQ ID NO: 1040 - protein, 6 aa, Artificial Sequence
  Synthetic peptide
VGSTNS

SEQ ID NO: 1041 - protein, 6 aa, Artificial Sequence
  Synthetic peptide
VNSTSG

SEQ ID NO: 1042 - protein, 6 aa, Artificial Sequence
  Synthetic peptide
VNSTSN

SEQ ID NO: 1043 - protein, 6 aa, Artificial Sequence
  Synthetic peptide
VSSQSQ

SEQ ID NO: 1044 - protein, 6 aa, Artificial Sequence
  Synthetic peptide
VSSQVQ

SEQ ID NO: 1045 - protein, 6 aa, Artificial Sequence
  Synthetic peptide
VSSTNG

SEQ ID NO: 1046 - protein, 6 aa, Artificial Sequence
  Synthetic peptide
VTSNSG

SEQ ID NO: 1047 - protein, 6 aa, Artificial Sequence
  Synthetic peptide
VTSQSQ

SEQ ID NO: 1048 - protein, 6 aa, Artificial Sequence
  Synthetic peptide
VEALYL

SEQ ID NO: 1049 - protein, 6 aa, Artificial Sequence
  Synthetic peptide
LYQLEN

SEQ ID NO: 1050 - protein, 6 aa, Artificial Sequence
  Synthetic peptide
GYVIIK

SEQ ID NO: 1051 - protein, 6 aa, Artificial Sequence
  Synthetic peptide
SNQNNF

SEQ ID NO: 1052 - protein, 6 aa, Artificial Sequence
  Synthetic peptide
SVLTSL

SEQ ID NO: 1053 - protein, 6 aa, Artificial Sequence
  Synthetic peptide
SSTNVG

SEQ ID NO: 1054 - protein, 6 aa, Artificial Sequence
  Synthetic peptide
SVSSSY

SEQ ID NO: 1055 - protein, 6 aa, Artificial Sequence
  Synthetic peptide
GAILSS

SEQ ID NO: 1056 - protein, 6 aa, Artificial Sequence
  Synthetic peptide
GAIIGL

SEQ ID NO: 1057 - protein, 6 aa, Artificial Sequence
  Synthetic peptide
AIIGLM

SEQ ID NO: 1058 - protein, 6 aa, Artificial Sequence
  Synthetic peptide
GGVVIA

SEQ ID NO: 1059 - protein, 6 aa, Artificial Sequence
  Synthetic peptide
SVNDLV

SEQ ID NO: 1060 - protein, 6 aa, Artificial Sequence
  Synthetic peptide
LKVKVL

SEQ ID NO: 1061 - protein, 6 aa, Artificial Sequence
  Synthetic peptide
NKGAII

SEQ ID NO: 1062 - protein, 6 aa, Artificial Sequence
  Synthetic peptide
NVSTSG

SEQ ID NO: 1063 - protein, 41 aa, Homo sapiens
AEFRHDSGYEVHHQKLVFFAEDVGSNKGAIIGLMVGGVVIA

SEQ ID NO: 1064 - protein, 40 aa, Homo sapiens
DAEFRHDSGYEVHHQKLVFFAEDVGSNKGAIIGLMVGGVV

SEQ ID NO: 1065 - protein, 21 aa, Homo sapiens
MEVGWYRSPFSRVVHLYRNGK

(19) United States
(12) Patent Application Publication
(10) Pub. No.: US 2020/0000871 A1
(43) Pub. Date: Jan. 2, 2020

(54) B-1A LYMPHOCYTE AND/OR MACROPHAGE TARGETING AND ACTIVATION TO TREAT MEDICAL CONDITIONS WITH INFLAMMATORY OR AUTOIMMUNE COMPONENTS

(71) Applicant: THE BOARD OF TRUSTEES OF THE LELAND STANFORD JUNIOR UNIVERSITY, Palo Alto, CA (US)

(72) Inventors: Lawrence Steinman, Palo Alto, CA (US); Michael Kurnellas, Palo Alto, CA (US); Jonathan Rothbard, Palo Alto, CA (US)

(73) Assignee: THE BOARD OF TRUSTEES OF THE LELAND STANFORD JUNIOR UNIVERSITY, Palo Alto, CA (US)

(21) Appl. No.: 16/572,301

(22) Filed: Sep. 16, 2019

Related U.S. Application Data
(63) Continuation of application No. 15/777,540, filed on May 18, 2018.
(60) Provisional application No. 62/256,814, filed on Nov. 18, 2015.

(51) Int. Cl. A61K 38/08; A61K 9/00

(52) U.S. Cl. CPC A61K 38/08; A61K 9/0073

(57) ABSTRACT

Methods and formulations to target and activate B-1a lymphocytes and/or macrophages are described. The methods and formulations can be delivered to the respiratory tract to target B-1a lymphocytes and/or macrophages in the pleural cavity. Conditions with inflammatory or autoimmune components can be treated. B-1a lymphocytes and/or macrophages can be targeted and activated using fibrils or fibril-forming peptides.

### CROSS REFERENCE TO RELATED APPLICATION

This application is a continuation of co-pending U.S. Utility application Ser. No. 15/777,540, filed May 18, 2018; which is the U.S. National Phase of PCT/US2016/062878, filed on Nov. 18, 2016; which claims priority to U.S. Provisional Patent Application No. 62/256,814 filed Nov. 18, 2015. The entire content of each of these prior applications is incorporated by reference herein.

### REFERENCE TO SEQUENCE LISTING

A computer readable text file, entitled “25Y7660.txt (Sequence Listing.txt)” created on or about Sep. 16, 2019, with a file size of 184 KB, contains the sequence listing for this application and is hereby incorporated by reference in its entirety.

### FIELD OF THE DISCLOSURE

Methods and formulations to target and activate B-1a lymphocytes and/or macrophages are described. The methods and formulations can be delivered to the respiratory tract to target B-1a lymphocytes and/or macrophages in the pleural cavity. Conditions with inflammatory or autoimmune components can be treated. B-1a lymphocytes and/or macrophages can be targeted and activated using fibrils or fibril-forming peptides.

### BACKGROUND OF THE DISCLOSURE

B cells of the immune system produce antibodies. Historically, all B cells were thought to produce antibodies following exposure to a pathogen. It is now known, however, that B2 cells produce antibodies following exposure to a pathogen while B1 cells produce antibodies in the absence of exposure to pathogen. B1 cells also differ functionally from B2 cells in efficiently presenting antigen to T cells, and in displaying evidence of tonic signaling in the absence of specific stimulation.

The antibodies produced by B1 cells (referred to as “natural” or “resting” immunoglobulins (e.g., resting IgM or IgA)) differ from other antibodies in that they are more broadly reactive and repertoire-selected. It is now known that resting immunoglobulins play an important role in early defense against bacterial and viral infections, and also play a role in a wide variety of diseases, through recognition of self-antigens and binding of cellular debris.

B1 cells can be further subdivided into B-1a lymphocytes and B-1b lymphocytes. Both types have the marker profile CD20⁺CD27⁺CD43⁺CD70⁻. B-1a lymphocytes are CD5⁺ while B-1b lymphocytes are CD5⁻. B-1a and B-1b precursor cells also differ in CD138 expression levels.

Macrophages are white blood cells produced by the division of monocytes. Monocytes and macrophages are phagocytes, and play a role in innate immunity (non-specific immune defenses) as well as helping to initiate adaptive immunity (specific defense mechanisms). These cells phagocytose (engulf and then digest) cellular debris and pathogens either as stationary or as mobile cells. When activated by pathogens or by other mechanisms, macrophages stimulate and recruit lymphocytes and other immune cells to respond to the insult.

Although macrophages play a vital role in host immune defenses, activated macrophages are also involved in the progression of a number of diseases and disorders. Activated macrophages elicit massive leukocyte infiltration and flood the surrounding tissue with inflammatory mediators, pro-apoptotic factors, and matrix degrading proteases. These actions can result in inflammation that can dismantle tissues to the point of inflicting serious injury. Tissue destruction perpetrated by macrophage-induced inflammation has been associated with the development of tumors, autoimmune disorders, and other conditions.

### SUMMARY OF THE DISCLOSURE

The current disclosure provides that targeting and activating B-1a lymphocytes and/or macrophages along the respiratory tract can treat medical conditions having inflammatory or autoimmune components. B-1a lymphocytes and/or macrophages can be effectively targeted and activated using fibrils or fibril-forming peptides.

### BRIEF DESCRIPTION OF THE FIGURES

FIGS. 1A-1C Amyloid fibrils composed of Tau 623-628 bind and are endocytosed by B-1a lymphocytes (CD19 CD5) and LPMs (CD11bhi F4/80hi peritoneal MΦs). (FIG. 1A) A composite of a confocal image (40× magnification) and (FIG. 1B) a single Z-cut (63×) from a movie constructed from the set of confocal images of peritoneal cavity cells from wild type mice injected with 10 μg FITC-Tau 623-628 and stained with rat anti-mouse CD19 (PE), F4/80 (Alexa Fluor 647), and DAPI. Cells were visualized using a Lei ca TCS SP8 white light laser confocal microscope. (FIG. 1C) Wild type mice were injected with 10 μg FITC-Tau 623-628 and peritoneal cells were isolated after 10 minutes and compared with peritoneal cells from an uninjected animal. The cells were washed and stained with rat anti-mouse CD11b (PE), CD19 (Pacific Blue), CD5 (APC), CD3 (PerCP-Cy5.5), and propidium iodide for viability. The gates demarcating the different cell types are shown in the two upper panels (10 minutes post-injection), and the amount of staining with FITC-Tau for each cell type is shown in the five lower panels.

FIGS. 2A-2D. Gating strategy used to identify the various murine peritoneal cell populations. Total cells from peritoneal lavage were stained with rat anti-mouse CD11b (PE), CD19 (Pacific Blue), CD5 (APC), CD3 (PerCP-Cy5.5), and propidium iodide (viability marker). (FIG. 2A) Peritoneal cells from unimmunized mice were sequentially gated to identify macrophages (CD11b), eosinophils (CD11b^{lo}SSC^{hi}), mast cells (CD11b⁻SSC^{hi}), T cells (CD11b⁻ CD5^{hi}CD19⁻), B-1a (CD19^{hi}CD5⁺) and B-2 (CD19⁺CD5⁻) cells. (FIG. 2B) The various peritoneal cell subsets from unimmunized mice were analyzed for their levels of auto-fluorescence emitted into the FITC channel to establish the negative threshold (i.e. FITC-Tau^{negative}), which was used to determine the binding of FITC-Tau shown in FIG. 1. (FIG. 2C) Total live peritoneal cells 10 min and 5 h after intraperitoneal injection of 10 μg FITC-Tau were processed and stained as described above and gated to identify macrophages, eosinophils, and mast cells. CD11b^{hi} macrophages are dramatically decreased 5 h post-injection, i.e. from >40% (left panel) to <3% (right panel). (FIG. 2D) Panels show peritoneal B cells (B-1 and B-2) 10 min and 5 h after FITC-Tau injection. Ten minutes after injection majority (>70%) of peritoneal B-1a and B-2 cells endocytose FITC-Tau (middle panel). In contrast, 5 h after injection the percentage of peritoneal B-1a and B-2 cells are dramatically decreased, and the remaining resident B-1a and B-2 cells are negative for FITC-Tau (right panel).

FIG. 3. Incubation of Amylin 28-33 with peritoneal cells in vitro does not affect cell viability. Peritoneal cells were isolated by lavage of C57BL/6 mice, aliquoted 500,000 cells per well of a 96 well round bottomed plate and treated with 1.0, 0.5, 0.25, 0.125 μg/well of Amylin 28-33 for 30 minutes or 120 minutes in DMEM with 10% FCS. At the end of the incubation the cells were spun, supernatant aspirated, and the cells were stained with rat anti-mouse CD11b (Pacific Blue), CD5 (PE), CD19 (APC), and incubated for 30 minutes on ice. The cells were washed once, aspirate transferred to FACS tubes, PI added, and analyzed by flow cytometry.

FIGS. 4A-4F. B-1a lymphocytes and IL-10 are necessary for therapeutic efficacy of amyloidogenic peptides. μMT mice were treated daily with intraperitoneal (i.p.) injections of 10 μg (FIG. 4A) Amylin 28-33 (n=10) or (FIG. 4B) Tau 623-628 (n=10) at onset of symptoms. (FIG. 4C) Wild type EAE mice were treated daily i.p. with 10 μg Amylin 28-33 (n=10). (FIG. 4D) IL-10 deficient (n=7) and (FIG. 4E) wild type (n=10) mice were treated daily with 10 μg Amylin 28-33. Values in graph represent mean+/−S.E.M. *p<0.05 and **p<0.005 by Mann-Whitney U test. All experiments were repeated at least twice. (FIG. 4F) Adoptive transfer of 3.5×10⁵ B-1a cells into μMT mice prior to the signs of EAE were treated daily i.p. with 10 μg Amylin 28-33 or control buffer (n=6). Mice without transfer of cells were treated with 10 μg Amylin 28-33. Values in graph represent mean+/−S.E.M. *p<0.05 by Mann-Whitney U test.

FIG. 5. Real time measurement of trafficking of adoptively transferred B-1a lymphocytes and LPMs using bioluminescence induced by amyloidogenic peptides. (FIG. 5, top left) 1×10⁵ and 2×10⁵ B-1a sorted cells were injected i.p. into C57BL/6 albino mice and the substrate luciferin, 5 minutes later bioluminescence images were obtained using CCD camera serially every 5 minutes. Bioluminescent signal was detectable from the peritoneum area diminishing with time and relocalizing in the inguinal lymph nodes area. (FIG. 5, top right) Quantification of B-1a Luc⁺ cell distribution by measuring light emission from the C57BL/6 albino mice over time after injection of Tau 623-628. (FIG. 5, bottom left) 1×10⁶ luc⁺ LPMs were injected i.p. into C57BL/6 albino mice and luciferase was re-injected every 30 minutes before an image was obtained. The MΦs egressed from the peritoneum (larger circle) and migrated to different tissues including the inguinal lymph nodes (smaller circle). (FIG. 5, bottom right) Quantification of MΦs cells migrating to the lymph nodes from the peritoneum by measuring the light emission. BLI measured from the lymph nodes increased by 10 fold at 60 min compared to the initial measurement at 5 min.

FIGS. 6A-6D. Amyloid fibrils, composed of either Tau 623-628 or Amylin 28-33, induce a different pattern of gene expression than LPS in B-1a lymphocytes and peritoneal MΦs, SPM and LPM. (FIG. 6A) Differential gene expression (720 annotated genes) expressed as a heatmap induced by LPS and the two types of amyloid fibrils. RNA isolated from purified B-1a lymphocytes and CD11b^{high} MΦs isolated from groups of three C57BL/6 mice injected with either 10 μg LPS, Amylin 28-33, Tau 623-628, or buffer. Each of the RNA samples was hybridized to a microarray plate (SurePrint G3 Mouse; Agilent Technologies) and quantified and analyzed using GeneSpring and Ingenuity software. Measurement by qPCR of gene induction compared to cells from uninjected animals of sets of genes representing (FIG. 6B) inflammatory cytokines, (FIG. 6C) immune suppressive genes, or (FIG. 6D) activation genes. Graphs represent the results of three separate measurements. The full set of data has been deposited in the Geo databank.

FIGS. 7A and 7B. Intranasal delivery of Amylin 28-33 reduces the clinical signs of EAE. (FIG. 7A) Mice with EAE were treated daily intranasally with 10 μg Amylin 28-33 (n=16) for 10 days at onset of symptoms. Values in graph represent mean+/−S.E.M. *p<0.05 and **p<0.005 by Mann-Whitney U test Experiments were repeated twice. (FIG. 7B) Splenocytes from EAE mice treated with 10 μg Amylin 28-33 were stimulated with 0, 5, 10 and 20 μg/ml MOG₃₅₋₅₅ and the levels of cytokines IL-6, IFNγ, IL-2, and IL-17 were measured (n=3). Values in graph represent mean+/−S.E.M. *p<0.01, **p<0.001, and **p<0.0001 by student's t test.

FIG. 8. Exemplary sequences of Aβ42 and Aβ40 (SEQ ID NOs: 1063 and 1064).

### DETAILED DESCRIPTION

B cells of the immune system produce antibodies. Historically, all B cells were thought to produce antibodies following exposure to a pathogen. It is now known, however, that B2 cells produce antibodies following exposure to a pathogen while B1 cells produce antibodies in the absence of exposure to pathogen. B1 cells also differ functionally from B2 cells in efficiently presenting antigen to T cells, and in displaying evidence of tonic signaling in the absence of specific stimulation.

The antibodies produced by B1 cells (referred to as “natural” or “resting” immunoglobulins (e.g., resting IgM or IgA)) differ from other antibodies in that they are more broadly reactive and repertoire-selected. It is now known that resting immunoglobulins play an important role in early defense against bacterial and viral infections, and also play a role in a wide variety of diseases, through recognition of self-antigens and binding of cellular debris.

B1 cells can be further subdivided into B-1a lymphocytes and B-1b lymphocytes. Both types have the marker profile CD20⁺CD27⁺CD43⁺CD70⁻. B-1a lymphocytes are CD5⁺ while B-1b lymphocytes are CD5-. B-1a and B-1b precursor cells also differ in CD138 expression levels.

Macrophages are white blood cells produced by the division of monocytes. Monocytes and macrophages are phagocytes, and play a role in innate immunity (non-specific immune defenses) as well as helping to initiate adaptive immunity (specific defense mechanisms). These cells phagocytose (engulf and then digest) cellular debris and pathogens either as stationary or as mobile cells. When activated by pathogens or by other mechanisms, macrophages stimulate and recruit lymphocytes and other immune cells to respond to the insult.

Although macrophages play a vital role in host immune defenses, activated macrophages are also involved in the progression of a number of diseases and disorders. Activated macrophages elicit massive leukocyte infiltration and flood the surrounding tissue with inflammatory mediators, pro-apoptotic factors, and matrix degrading proteases. These actions can result in inflammation that can dismantle tissues to the point of inflicting serious injury. Tissue destruction perpetrated by macrophage-induced inflammation has been associated with the development of tumors, autoimmune disorders, and other conditions.

The current disclosure provides that targeting and activating B-1a lymphocytes and/or macrophages along the respiratory tract can treat medical conditions having inflammatory or autoimmune components. B-1a lymphocytes and/or macrophages can be effectively targeted and activated using fibrils or fibril-forming peptides.

The respiratory tract is the structure involved in the exchange of gases between the atmosphere and the blood stream. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which lead to the ultimate respiratory zone, the alveoli or deep lung where the exchange of gases occurs. The alveolar surface area is the largest in the respiratory system where active agent (e.g., drug) absorption occurs.

The pleura is a membrane which surrounds the lungs and has a two-layer structure including an outer or parietal pleura that is normally attached to the chest wall and an inner or visceral pleura that covers the lungs and adjoining structures. The space between the inner and outer pleurae is referred to as the pleural cavity, pleural space, or intrapleural space.

Particular embodiments disclosed herein include administration of formulations including active agents to various portions of the respiratory tract. Particular embodiments include administration to the respiratory tract to target and activate B-1a lymphocytes and/or macrophages in the pleural cavity.

Pulmonary administration refers to administration of formulations so that they reach the lungs and in particular embodiments the alveolar regions of the lung. In particular embodiments, pulmonary administration occurs by inhalation or administration through the nose or mouth.

The geometry of the airways is a major barrier for active agent dispersal within the lungs. There are three basic mechanisms of deposition: impaction, sedimentation, and Brownian motion (Padfield. 1987. In: D. Ganderton & T. Jones eds. Drug Delivery to the Respiratory Tract, Ellis Harwood, Chicherster, U.K.). Impaction occurs when particles are unable to stay within the air stream, particularly at airway branches. They are adsorbed onto the mucus layer covering bronchial walls and cleaned out by mucocilliary action. Impaction most often occurs with particles over 5 μm in diameter. Smaller particles (<5 μm) stay within the air stream more readily and can be transported deep into the lungs. Sedimentation often occurs in the lower respiratory system where airflow is slower. Very small particles (<0.6 μm) can deposit by Brownian motion. In particular embodiments, this deposition is undesirable because it is not targeted to the alveoli (Worakul & Robinson. 2002. In: Polymeric Biomaterials, 2^{nd} ed. S. Dumitriu ed. Marcel Dekker. New York).

Formulations can be delivered to various portions of the respiratory tract in, for example, droplets, dry powder forms, foams, gels, mists, particles, solutions, sprays, suspensions, and/or vapors. In particular embodiments, formulations can be aerosolized.

An aerosol refers to any preparation of a fine mist of particles, typically less than 10 microns in diameter. In particular embodiments, the mean diameter for aqueous formulations of aerosol particles is, for example between 0.1 and 30 microns, between 0.5 and 20 microns, between 0.5 and 10 microns or 5 microns.

Aerosols for the delivery of active agents to the respiratory tract are described in, for example, Adjei and Garren, J. Pharm. Res., 7: 565-569 (1990); Zanen and Lamm, J. Int. J. Pharm., 114: 111-115 (1995); Gonda, Critical Reviews in Therapeutic Drug Carrier Systems, 6:273-313 (1990); and Moren, “Aerosol dosage forms and formulations,” in: Aerosols in Medicine, Principles, Diagnosis and Therapy, Moren, et al., Eds. Esevier, Amsterdam, 1985.

Aerosols are typically formed by atomizing a solution or suspension under pressure through a nebulizer, a pressurized can, a continuous sprayer, or through the use of a metered dose inhaler (“MDI”) or pressurized metered dose inhaler (pMDI).

Nebulizers create a fine mist from a solution or suspension, which is inhaled by a subject. Nebulized saline solutions have long been delivered chronically to the lungs with small amounts of active agents, such as beta agonists, corticosteroids, or antibiotics. Often these saline solutions are hypertonic (sodium chloride concentrations greater than 0.9% by weight, often as high as 5% by weight) and generally they are delivered for up to 20 minutes. VENTOLIN® Inhalation Solution (GSK) is an albuterol sulfate solution that can be nebulized for administration to the lungs. A VENTOLIN® solution for nebulization can be prepared by mixing, for example, 1.25-2.5 mg of albuterol sulfate in 0.25-0.5 mL of aqueous solution into sterile normal saline to achieve a total volume of 3 mL. No adverse effects have been found to be associated with the delivery to the lungs by VENTOLIN® nebulization. Nebulizing devices are described in, for example, U.S. Pat. No. 5,709,202.

pMDIs typically include a pressurized canister having a meter valve, wherein the canister is filled with a solution or suspension and a propellant. In particular embodiments, the solution or suspension acts as the propellant. In other embodiments, propellants can include hydrofluoroalkane (HFA) propellants (e.g., Proventil® HFA (Schering-Plough Corporation) or FREON® (E. I. Du Pont De Nemours and Co. Corp.). When released from the canister, the formulation is a fine mist, and the propellant and solvent may wholly or partially evaporate due to the decrease in pressure.

Dry powder formulations can also be used. Dry powder formulations (DPFs) with large particle size have improved flowability characteristics, such as less aggregation, easier aerosolization, and potentially less phagocytosis. Dry powder aerosols for inhalation are generally produced using particles with mean diameters primarily in the range of less than 5 microns, although in particular embodiments, the range is between one and ten microns in aerodynamic diameter. Large “carrier” particles (containing no active agent) have been co-delivered with dry powder aerosols to aid in achieving efficient aerosolization among other possible benefits. Particles with degradation and release times ranging from seconds to months can be designed and fabricated by methods known in the art.

In particular embodiments, “aerodynamically light particles” can be used in dry powder formulations. “Aerodynamically light particles” are particles having a mean or tap density less than 0.4 g/cm³. Tap density is a standard measure of the envelope mass density. The envelope mass density of an isotropic particle is defined as the mass of the particle divided by the minimum sphere envelope volume in which it can be enclosed. Features contributing to low tap density include irregular surface texture and porous structure. The tap density of particles of a dry powder may be obtained by the standard USP tap density measurement.

The currently preferred mean diameter for aerodynamically light particles for inhalation is between 3 and 30 microns in diameter, or between 5 and 7 microns. The aerodynamically light particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agent may be administered to target different regions of the lung in one administration.

In particular embodiments, conductive formulations can be used for administration to the respiratory tract. Conductive formulations are particularly useful to suppress particle exhalation. Solution conductivity is a product of the ionic strength, concentration, and mobility (the latter two contribute to the conductivity of the formulation as a whole). Any form of ionic components (anionic, cationic, or zwitterionic) can be used. These conductive materials may alter the properties of the mucosal lining of the respiratory tract by acting, for example, as a cross-linking agent within the mucus. The ionic components in conductive formulations can interact with the strongly linked anionic glycoproteins within normal tracheobronchial mucus. These interactions influence the state of the air/liquid surface of the airway lining fluid and transiently the nature of the physical entanglements due to covalent and noncovalent interactions, including hydrogen bonding, hydrophobic, and electrostatic interactions.

Substances useful to form conductive formulations are those that are easily ionized in an aqueous or organic solvent environment (also referred to herein as “conductive agents”) Examples of such substances include salts, ionic surfactants, charged amino acids, charged proteins, or other charged materials.

Suitable salts include any salt form of sodium, potassium, magnesium, calcium, aluminum, silicon, scandium, titanium, vanadium, chromium, cobalt, nickel, copper, manganese, zinc, tin, and similar elements. Examples include sodium chloride, sodium acetate, sodium bicarbonate, sodium carbonate, sodium sulfate, sodium stearate, sodium ascorbate, sodium benzoate, sodium biphosphate, sodium phosphate, sodium bisulfite, sodium citrate, sodium borate, sodium gluconate, calcium chloride, calcium carbonate, calcium acetate, calcium phosphate, calcium alginite, calcium stearate, calcium sorbate, calcium sulfate, calcium gluconate, magnesium carbonate, magnesium sulfate, magnesium stearate, magnesium trisilicate, potassium bicarbonate, potassium chloride, potassium citrate, potassium borate, potassium bisulfite, potassium biphosphate, potassium alginate, potassium benzoate, magnesium chloride, cupric sulfate, chromium chloride, stannous chloride, and sodium metasilicate.

Suitable ionic surfactants include sodium dodecyl sulfate (SDS) (also known as sodium lauryl sulfate (SLS)), magnesium lauryl sulfate, Polysorbate 20, Polysorbate 80, and similar surfactants.

Suitable charged amino acids include L-lysine. L-arginine, histidine, aspartate, glutamate, glycine, cysteine, and tyrosine.

Suitable charged proteins include calmodulin (CaM), troponin C, and charged phospholipids. Negatively charged phospholipids include phosphatidylinositol, phosphatidylserine, phosphatidylglycerol, phosphatidic acid, cardiolipins, dialkanoyl phosphatidyl glycerols (dipalmitoyl phosphatidyl glycerol and dimyristoyl phosphatidyl glycerol), phosphatidylinositol 4-phosphate (PIP), phosphatidylinositol 4,5-bisphosphate (PIP2), and phosphatidylethanolamines. Positively charged phospholipids include dioleoyl trimethylammonium propane, esters of phosphatidic acids, dipalmitoylphosphatidic acid, and distearoyl-phosphatidic acid with aminoalcohols such as hydroxyethylenediamine.

In particular embodiments, conductive formulations can have conductivity values of greater than 5,000 μS/cm, greater than 10,000 μS/cm, or greater than 20,000 μS/cm. In particular embodiments, conductive formulations can have conductivity values within ranges of 4,000-50,000 μS/cm; 10,000-40,000 μS/cm; 30,000-60,000 μS/cm. In particular embodiments, conductive formulations have a specific conductivity that is greater than the specific conductivity of isotonic saline.

Particular embodiments of conductive formulations include salts, such as saline (0.15 M NaCl or 0.9%) solution, CaCl₂ solution, CaCl₂ in saline solution, or saline solution containing ionic surfactants, such as SDS or SLS. In particular embodiments, the formulation includes saline solution and CaCl₂. Suitable concentration ranges of the salt or other conductive/charged compounds can vary from 0.01% to 20%, 0.1% to 10% or 0.1 to 7% (weight of conductive or charged compound/total weight of formulation). In particular embodiments, the formulation contains a hypertonic saline solution (i.e. sodium chloride concentration greater than 0.9% by weight).

In particular embodiments, the formulations include a mucoactive or mucolytic agent, which is a substance that can modify mucus production, secretion, composition and/or interactions with the epithelium. Examples of mucoactive or mucolytic agents include MUCSAC and MUC5B mucins, DNA, N-acetylcysteine (NAC), cysteine, nacystelyn, domase alfa, gelsolin, heparin, heparin sulfate. P2Y2 agonists (e.g. UTP, INS365), and nedocromil sodium.

Targeting and activating B-1a lymphocytes and/or macrophages (e.g., pleural cavity B-1a lymphtocytes and/or macrophages) through administration of formulations to the respiratory tract can treat numerous medical conditions with inflammatory or autoimmune components.

Conditions with inflammatory or autoimmune components include allergic airway disease, Alpers' disease, Alzheimer's disease (AD), amyloid nephropathy, amyloid neuropathy, amyotrophic lateral sclerosis (ALS), asthma, Batten disease, cancer, cardiac ischemia-reperfusion injury, celiac disease, cerebro-oculo-facio-skeletal syndrome, chemotherapy-associated cognitive impairment and dementia, chronic hepatitis, chronic inflammatory demyelinating polyneuropathy, chronic obstructive pulmonary disease (COPD), collagen induced arthritis, corticobasal degeneration, Creutzfeldt-Jakob disease, Crohn's disease, depression-induced dementia, Friedreich's ataxia, frontotemporal dementia, Gerstmann-Straussler-Scheinker disease, glaucoma, Graves' disease, HIV-Related cognitive impairment, Huntington's disease (HD), immune thrombocytopenia purpura, inflammatory bowel disease, insulin resistance, ischemia/reperfusion injury, juvenile arthritis, Lacunar syndromes, Lewy body disease, lupus, macular degeneration, mild cognitive impairment, monomelic amyotrophy, motor neuron diseases (MND), multiple sclerosis, multiple system atrophy, multiple system atrophy with orthostatic hypotension (Shy-Drager syndrome), myasthenia gravis, neurodegeneration with brain iron accumulation, opsoclonus myoclonus, Parkinson's disease (PD), post-encephalitic dementia, post-traumatic stress disorder, syndromeposterior cortical atrophy, prion diseases, primary antiphospholipid syndrome, primary progressive aphasia, primary Sjogren's syndrome, progressive multifocal leukoencephalopathy, progressive supranuclear palsy, pseudodementia, retinal ischemia-reperfusion injury, retinitis pigmentosa, rheumatoid arthritis, spinal cord injury, spinal muscular atrophy (SMA), spinocerebellar ataxia (SCA), stroke, systemic lupus erythematosus, traumatic brain injury, type 1 diabetes, type 2 diabetes, vascular dementia, and Wernicke-Korsakoffs syndrome.

Ischemia, for example, is a restriction in blood supply that causes tissue damage in affected areas due to insufficient supply of oxygen and glucose to maintain cellular metabolism. Inadequate blood flow can be caused by vasoconstriction, artery blockage, low blood pressure, septic shock, anemia, heart failure or organ transplant. Depending on the type of tissue affected, irreversible damage may take occur within 3-5 minutes (e.g., in the brain or heart) or within 10-20 minutes in less aerobically intense organs (e.g., skin). Ischemia leads to the buildup of metabolic waste and cell leakage, and symptoms may include angina, inflammation, mottling or discoloration of skin.

After ischemia, reperfusion injury can occur when blood flow is reintroduced, where the return of oxygen can lead to overproduction of free radicals and reactive oxygen species (ROS), and causing oxidative damage to tissues, for example caused by Nox2 activity. Reperfusion can lead to cardiac arrhythmia, accelerated cell self-destruction, and exaggerated inflammation of tissue already inflamed due to ischemia as white blood cells overreact to tissue damage.

Asthma is a respiratory disease in which bronchial inflammation triggered by an allergic reaction or infection with a bacterium or virus becomes chronic to thereby cause increased airway hyperresponsiveness and reversible airway narrowing, leading to symptoms such as attacks of wheezing, cough, apnea, and chest tightness. These symptoms are more common at night or in the early morning. As the responsiveness of the airway increases, the symptoms become more severe and continuous, and daily variation in pulmonary function increases.

Chronic obstructive pulmonary disease (COPD) is a chronic lung disease that leads to breathing difficulties. It can be chronic bronchitis (a long term, productive cough) or emphysema (lung destruction), or a combination of both. It is most commonly caused by smoking, with other risk factors including inhalation of gases or fumes, exposure to secondhand smoke, or high levels of pollution. Symptoms include cough, fatigue, respiratory infections, shortness of breath, and wheezing.

Cancer (neoplasia) is characterized by deregulated cell growth and cell division. Examples of cancer with inflammatory components include acoustic neuroma, adenocarcinoma, astrocytoma, basal cell cancer, bile duct cancer, bladder cancer, brain cancer, breast cancer, bronchogenic cancer, central nervous system cancer, cervical cancer, chondrosarcoma, choriocarcinoma, chronic lymphocytic leukemia, colon cancer, craniopharyngioma, ependymoma, Ewing's tumor, fibrosarcoma, glandular cancer, glioma, hairy cell leukemia, hemangioblastoma, hepatocellular carcinoma, hepatoma, kidney cancer, leiomyosarcoma, liver cancer, liposarcoma, lung cancer, melanoma, medulloblastoma, medullary cancer, medullary thyroid cancer, menangioma, mesothelioma, myxosarcoma, neuroblastoma, non-Hodgkin's lymphoma, oligodendroglioma, osteogenic sarcoma, ovarian cancer, papillary adenocarcinomas, papillary thyroid cancer, pancreatic cancer, pheochromocytomas papillary cancer, pineal cancer, prolymphocytic leukemia, prostate cancer, renal cell cancer, retinoblastoma, rhabdomyosarcoma, sarcoma, sebaceous gland cancer, seminoma, skin cancer, squamous cell cancer, sweat gland cancer, synovioma, testicular cancer, and/or Wilms' tumor.

Effective treatments against cancer can decrease the number of cancer cells, decrease the number of metastases, decrease tumor volume, induce apoptosis of cancer cells, induce cancer cell death, induce chemo- or radio-sensitivity in cancer cells, inhibit angiogenesis near cancer cells, inhibit cancer cell proliferation, and/or inhibit tumor growth. Effective treatments against cancer can also increase life expectancy, prolong a subject's life, reduce cancer-associated pain, and/or reduce relapse or re-occurrence of the cancer following treatment. In particular embodiments effective treatments against cancer prevent, reduce, or delay the number or severity of metastatic tumors.

Rheumatoid arthritis is a chronic syndrome characterized by usually symmetric inflammation of the peripheral joints, potentially resulting in progressive destruction of articular and periarticular structures, with or without generalized manifestations. Onset of rheumatoid arthritis is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

Multiple sclerosis (MS) is an inflammatory neurological disease characterized by various symptoms and signs of CNS dysfunction, with remissions and recurring exacerbations. The most common presenting symptoms are paresthesia in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

Relapsing remitting MS (RR MS) is characterized clinically by relapses and remissions that occur over months to years, with partial or full recovery of neurological deficits between attacks. Such patients manifest 1 attack, or relapse, per year. Over 10 to 20 years, 50% of RR MS patients develop secondary progressive MS (SP MS) which is characterized by incomplete recovery between attacks and accumulation of neurologic deficits resulting in increasing disability.

Diagnosis of MS is indirect, by deduction from clinical, radiographic (brain plaques on magnetic resonance [MR] scan), and to a lesser extent laboratory (oligoclonal bands on CSF analysis) features.

Huntington's disease (HD) is a neurodegenerative and inflammatory genetic disorder that affects muscle coordination and leads to cognitive decline and psychiatric problems. Clinical symptoms of HD include abnormal and/or unusual movements, anxiety, behavioral disturbances, chorea, cognitive impairment, confusion, difficulty swallowing, disorientation, fidgeting, hallucinations, head turning to shift eye position, involuntary grimaces, jerking movements of the arms, legs, face, and other body parts, irritability, lack of coordination, personality changes, memory loss, moodiness, paranoia, psychosis, restlessness, rigidity, small unintentionally initiated or uncompleted motions, slow movements, speech changes, speech impairment, suicidal thoughts, and suicide attempts, tremor, and weight loss.

Parkinson's disease (PD) is a degenerative and inflammatory disorder of the central nervous system. Four motor symptoms are considered hallmarks of PD: tremor, rigidity, slowness of movement, and postural instability. Later in disease progression, thinking and behavioral problems may arise and can range from mild to severe, with dementia commonly occurring in the advanced stages of the disease. Depression is the most common psychiatric symptom. Other common symptoms include disorders of speech, cognition, mood, behavior, and thought. Cognitive disturbances further include executive dysfunction, which can include problems with planning, cognitive flexibility, abstract thinking, rule acquisition, initiating appropriate actions and inhibiting inappropriate actions, selecting relevant sensory information, fluctuations in attention, slowed cognitive speed, and memory loss. Other symptoms include sleep disturbances.

Amyotrophic lateral sclerosis (ALS) is a fatal motor neuron disease, affecting both the first and second order motor neurons. Early clinical symptoms of ALS are typically weakness and/or muscle atrophy. Other early symptoms include trouble swallowing (dysphagia), cramping, or stiffness of affected muscles; muscle weakness affecting an arm or a leg; and/or slurred and nasal speech or trouble forming words (dysarthria), and in some cases dementia. Twitches of muscles that can be seen under the skin (fasciculations) are observed, and an abnormal reflex commonly called Babinski's sign indicates upper motor neuron damage.

The rate of progression of ALS can be measured using an outcome measure called the “ALS Functional Rating Scale Revised (ALSFRS-R)”, a 12-item instrument administered as a clinical interview or patient-reported questionnaire that produces a score between 48 (normal function) and 0 (severe disability). A survey-based study amongst clinicians showed that they rated a 20% change in the slope of the ALSFRS-R clinically meaningful (Castrillo-Viguera, et al., Amyotroph Lateral Scler, 11(1-2):178-80 (2010)).

Traumatic brain injury (TBI) occurs when an external mechanical force, typically head trauma, causes brain dysfunction. TBI can have wide-ranging physical and psychological effects. Some symptoms appear immediately while others may not appear until days or weeks after the traumatic event. Symptoms of TBI include loss of consciousness; a state of being dazed, confused or disoriented; memory or concentration problems; headache, dizziness or loss of balance; nausea or vomiting; sensory problems such as blurred vision, ringing in the ears or a bad taste in the mouth; sensitivity to light or sound; mood changes or mood swings; feeling depressed or anxious; fatigue or drowsiness; difficulty sleeping; sleeping more than usual, agitation, combativeness or other unusual behavior; slurred speech; inability to awaken from sleep; weakness or numbness in fingers and toes; loss of coordination; convulsions or seizures, dilation of one or both pupils of the eyes; and/or clear fluids draining from the nose or ears. In children, additional symptoms include change in eating or nursing habits; persistent crying and inability to be consoled; unusual or easy irritability; change in ability to pay attention; change in sleep habits; sad or depressed mood; and/or loss of interest in favorite toys or activities.

Effective treatments against conditions with an inflammatory or autoimmune component described herein can be identified by observing a statistically-significant improvement in a symptom associated with the condition in a clinical and/or research setting. For conditions where particular symptoms are not described herein, any clinically-relevant model of the condition known and accepted by clinicians and researchers in the relevant field can be used to assess the effectiveness of a treatment.

In particular embodiments, inflammatory or autoimmune conditions with a central nervous system component can be evaluated using tests for cognitive impairment, and/or neuropsychiatric morbidities, such as disorders of cognitive function, memory, mood, behavior, thought, REM Sleep Behavior Disorder, apathy, fatigue, indifference and lack of social engagement, and dullness. Methods of measuring and monitoring these aspects are known in the art and include, for example, serial position testing which focuses on human memory processes (Surprenant, Perception and Psychophysics, 63(4): 737-745 (2001)), word superiority testing which focuses on human speech and language (Krueger, Memory & Cognition, 20(6):685-694 (1992)), the Brown-Peterson test which focuses on human short-term memory (Naime, et al., Quarterly Journal of Experimental Psychology A: Human Experimental Psychology, 52:241-251 (1999)), memory span testing (May, et al., Memory & Cognition, 27(5):759-767 (1999)), visual search testing (Wolfe, et al., Journal of Experimental Psychology: Human Perception and Performance, 15(3):419-433 (1989)), and knowledge representation (e.g., semantic network) testing. Additional tests examine processing speed, reaction time, i.e. clock speed; flexibility and ability to adapt to changes in task rules; attention, focus and concentration; problem solving; memory; and verbal fluency. Representative tests and instruments include traditional IQ tests like the WAIS and Progressive Ravens Matrices, and the battery of tests available through Luminosity (Lumos Labs, Inc.).

In particular embodiments, medical conditions with inflammatory or autoimmune components can be treated by targeting and activating B-1a lymphocytes and/or macrophages. In particular embodiments, targeting and activation of B-1a lymphocytes and/or macrophages is evidenced by an increase in IL-10 expression by the B-1a lymphocytes and/or macrophages. IL-10 is an anti-inflammatory cytokine which can inhibit the production of proinflammatory cytokines.

In particular embodiments targeting and activating B-1a lymphocytes and/or macrophages leads to suppression of proinflammatory cytokines. Examples of proinflammatory cytokines include IL-6, TNF-α, IL-2, and IFN-γ. Suppression of proinflammatory cytokines can mean a statistically significant reduction in the expression of one or more proinflammatory cytokines.

In particular embodiments targeting and activation of B-1a lymphocytes and/or macrophages is evidenced by migration of the B-1a lymphocytes and/or macrophages to lymph nodes. In particular embodiments, targeting and activation of B-1a lymphocytes and/or macrophages is evidenced by an increase in IL-10 expression and migration of the B-1a lymphocytes and/or macrophages to lymph nodes. In particular embodiments, targeting and activation of B-1a lymphocytes and/or macrophages can be evidence by decreased production of proinflammatory cytokines. In particular embodiments, targeting and activation of B-1a lymphocytes and/or macrophages is evidenced by a statistically-significant reduction in a symptom associated with a medical condition.

In particular embodiments B-1a lymphocytes and/or macrophages are targeted and activated using fibrils or fibril forming peptides. In particular embodiments, a fibril refers to an aggregation of proteins or peptides into a fibrous formation, whereby many copies of the proteins or peptides attach to one another to form insoluble fibers. In particular embodiments a fibril forming peptide can be referred to as amyloidogenic. Examples of proteins and/or peptides that naturally form fibrils include amyloid beta, tau, and amylin.

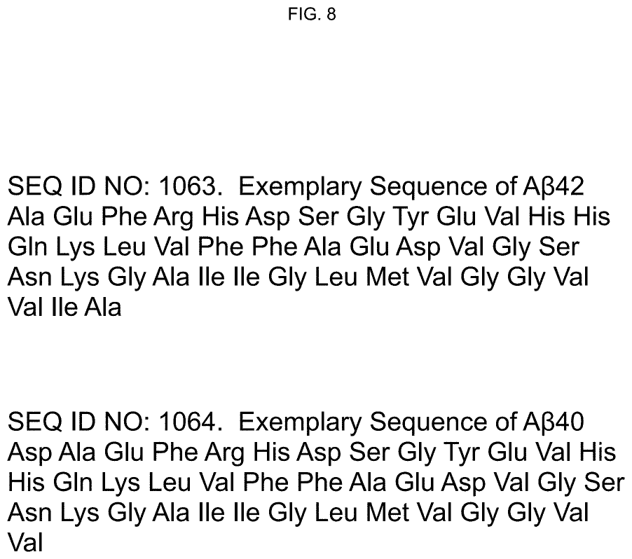

In particular embodiments, fibrils or fibril-forming peptides can include amyloid beta (An) peptides. Aβ peptides include the sequences set forth in SEQ ID NO: 1063 and SEQ ID NO: 1064 (FIG. 8) and fragments and derivatives thereof. Aβ is the main component of amyloid plaques. Aβ is formed after sequential cleavage of the amyloid precursor protein (APP), a transmembrane glycoprotein of undetermined function. APP can be processed by α-, β- and γ-secretases; Aβ protein is generated by successive action of the β- and γ secretases. The γ secretase, which produces the C-terminal end of the Aβ peptide, cleaves within the transmembrane region of APP and can generate a number of isoforms of 36-43 amino acid residues in length. The most common isoforms are Aβ 1-40 and Aβ 1-42 (“Aβ40” and “Aβ42”); the shorter form is typically produced by cleavage that occurs in the endoplasmic reticulum, while the longer form is produced by cleavage in the trans-Golgi network. Aβ40 is more common of the two, but Aβ42 is more fibrillogenic.

In particular embodiments, fibrils or fibril-forming peptides can be active fragments of Aβ. Active fragments of Aβ peptide share a functional or binding property with full length Aβ peptide. In particular embodiments an active fragment of Aβ can be any fragment of the full-length Aβ that can self-polymerize to form fibrils or aggregates. In particular embodiments an active fragment of Aβ can be any length from 5 to 43 amino acids in length. Epitopic fragments of Aβ peptides are fragments that retain the ability to bind to one or more anti-An monoclonal antibody. Aβ is intrinsically unstructured, meaning that in solution it does not acquire a compact tertiary fold but rather populates a set of structures. By NMR-guided simulations, Aβ40 and Aβ42 also seem to feature highly different conformational states, with the C-terminus of Aβ42 being more structured than Aβ40.

In particular embodiments, fibrils or fibril-forming peptides can be active fragments of amylin. Amylin is a peptide hormone that is secreted with insulin from the pancreas. In particular embodiments, the fibrils or fibril forming peptides can be residues 28-33 of the amylin protein, known as amylin 28-33 (SEQ ID NO: 1053). In particular embodiments, the fibrils or fibril-forming peptides can be amyloidogenic analogs of amylin 28-33. Amyloidogenic analogs of amylin 28-33 can include SEQ ID NO: 1062 and SEQ ID NO: 449 (Kumellas, et al. (2014) JEM 211(9): 1847).

In particular embodiments, fibril formation or aggregation of a peptide or protein can be measured using techniques including the thioflavin T assay, dynamic light scattering, and size exclusion chromatography. The thioflavin T assay can be used to measure fibril formation by treating a protein/peptide sample with thioflavin T, and then measuring fluorescence by microscopy or spectroscopy.

In particular embodiments, fibril refers to a fibrous aggregate of four or more peptide molecules linked through non-covalent bonds. In particular embodiments, the aggregates include one hundred or more, one thousand or more, five thousand or more, or ten thousand or more peptide molecules. In particular embodiments a fibril-forming or amyloidogenic peptide is a peptide that, either spontaneously or upon exposure to certain conditions such as temperature or salinity, oligomerizes through non-covalent interactions to form fibrils.

In particular embodiments, fibril-forming peptides include hexapeptides with amino acids of L-configuration, D-configuration or a mixture of configurations. In particular embodiments, fibril-forming peptides are peptides that generate a Rosetta energy of binding of −23 kcal/mol or less (i.e., more negative). Rosetta is a software suite that is used for computational modeling of proteins and peptides. The cut-off value of −23 kcalmol for fibril-forming potential is based on an experimentally validated model (see Goldschmidt et al (2010) PNAS 107(5): 3487-3492, for methods of Rosetta energy calculation for the hexapeptides).

In particular embodiments the peptides include 1, 2, or 3 amino acids having polar basic side chains. The polar basic side chains may have a terminal amino or a terminal imidazole group. In other cases, the peptides include 1 polar acidic side chain. In other cases, the peptides include 0, 1, 2, 3, 4, or 5 amino acids having hydrophobic side chains. In still other cases, the peptides include 0, 1, 2, 3, 4, or 5 amino acids having polar uncharged side chains, wherein the peptide has a positive charge. In particular embodiments, the fibrils or fibril-forming peptides do not contain proline.

The carboxy terminus of the peptides is typically either a carboxylic acid (i.e., —COOH) or an amide (i.e., —C(O)NR₂, where R is a substituent such as alkyl or hydrogen). The amino terminus is typically either an amine (i.e., N(R)₂, where R′ is a substituent such as alkyl or hydrogen) or an acetate group (i.e., —C(O)R″, where R″ is a substituent such as methyl, ethyl, or longer alkyl).

In particular embodiments, fibril-forming peptides may include one or more of the following hexapeptides, where each indicated amino acid is either an L-amino acid or a D-amino acid (symbol for hexapeptide indicated as its SEQ ID NO: identifier in parenthesis after the hexamer): SVNVDL (SEQ ID NO: 1); SLNVDV (SEQ ID NO: 2); SVDVNL (SEQ ID NO: 3); DLSWL (SEQ ID NO: 4); SVNLDV (SEQ ID NO: 5); SWNDV (SEQ ID NO: 6); DVSLVN (SEQ ID NO: 7); DVSVLN (SEQ ID NO: 8); SDLVNV (SEQ ID NO: 9); SLNWS (SEQ ID NO: 10); LNVDSV (SEQ ID NO: 11); NDLSW (SEQ ID NO: 12); VDNLVS (SEQ ID NO: 13); VNDVSL (SEQ ID NO: 14); VSDNVL (SEQ ID NO: 15); LNDWS (SEQ ID NO: 16); LSVDVN (SEQ ID NO: 17); NSVDLV (SEQ ID NO: 18); VDVLNS (SEQ ID NO: 19); VNDSVL (SEQ ID NO: 20); VNVSLD (SEQ ID NO: 21); LDVNSV (SEQ ID NO: 22); LNWDS (SEQ ID NO: 23); LSDWN (SEQ ID NO: 24); LWNDS (SEQ ID NO: 25); NSLDW (SEQ ID NO: 26); VLVDNS (SEQ ID NO: 27); VNLSDV (SEQ ID NO: 28); VNVDLS (SEQ ID NO: 29); DNVSVD (SEQ ID NO: 30); LSDNW (SEQ ID NO: 31); LSWDN (SEQ ID NO: 32); VLDVSN (SEQ ID NO: 33); VNDLVS (SEQ ID NO: 34); VNVSDL (SEQ ID NO: 35); WLSDN (SEQ ID NO: 36); LVSVNL (SEQ ID NO: 37); LVNVSV (SEQ ID NO: 38); SLNVSV (SEQ ID NO: 39); LVSVNS (SEQ ID NO: 40); SVDVNV (SEQ ID NO: 41); LWSVL (SEQ ID NO: 42); VNLWS (SEQ ID NO: 43); SNLVSV (SEQ ID NO: 44); SVNVLS (SEQ ID NO: 45); VLVSVL (SEQ ID NO: 46); LVNVSL (SEQ ID NO: 47); SVNVDS (SEQ ID NO: 48); VLSVNV (SEQ ID NO: 49); NLWSV (SEQ ID NO: 50); SWLNV (SEQ ID NO: 51); LSWNL (SEQ ID NO: 52); SNLWS (SEQ ID NO: 53); SWLDV (SEQ ID NO: 54); LVSLNV (SEQ ID NO: 55); VSLNW (SEQ ID NO: 56); VKVQIY (SEQ ID NO: 57); QWIYK (SEQ ID NO: 58); KVIQVY (SEQ ID NO: 59); VYVKIY (SEQ ID NO: 60); QIWYK (SEQ ID NO: 61); QVIKVY (SEQ ID NO: 62); VQVKIY (SEQ ID NO: 63); QWKIY (SEQ ID NO: 64); QVIVYK (SEQ ID NO: 65); QIVKVY (SEQ ID NO: 66); QKIVVY (SEQ ID NO: 67); QKVVYI (SEQ ID NO: 68); KVQVYI (SEQ ID NO: 69); QWKYI (SEQ ID NO: 70); KVQIYV (SEQ ID NO: 71); VKIQVY (SEQ ID NO: 72); VIQKVY (SEQ ID NO: 73); KWIYK (SEQ ID NO: 74); QIVKYV (SEQ ID NO: 75); QKVIYV (SEQ ID NO: 76); KQWIY (SEQ ID NO: 77); KIQVYV (SEQ ID NO: 78); KVYVQI (SEQ ID NO: 79); WQIYK (SEQ ID NO: 80); KQVIVY (SEQ ID NO: 81); VQIKVY (SEQ ID NO: 82); QKIVYV (SEQ ID NO: 83); VIQVYK (SEQ ID NO: 84); KWIQY (SEQ ID NO: 85); KVQVIY (SEQ ID NO: 86); QYWIK (SEQ ID NO: 87); YWIQK (SEQ ID NO: 88); KIWQY (SEQ ID NO: 89); YQVIVK (SEQ ID NO: 90); KIYVQV (SEQ ID NO: 91); KWIYQ (SEQ ID NO: 92); KYWQI (SEQ ID NO: 93); YQIWK (SEQ ID NO: 94); YWIQY (SEQ ID NO: 95); KQVIYV (SEQ ID NO: 96); KYVQVI (SEQ ID NO: 97); KYWIQ (SEQ ID NO: 98); QKWIY (SEQ ID NO: 99); QYVIVK (SEQ ID NO: 100); IVQKVY (SEQ ID NO: 101); KYIWQ (SEQ ID NO: 102); QVIKYV (SEQ ID NO: 103); YIWQK (SEQ ID NO: 104); YQWIK (SEQ ID NO: 105); IKVQVY (SEQ ID NO: 106); KWQYI (SEQ ID NO: 107); KYVQIV (SEQ ID NO: 108); VKQVYI (SEQ ID NO: 109); VQVIYK (SEQ ID NO: 110); WIQKY (SEQ ID NO: 111); IQVVYK (SEQ ID NO: 112); KIVVYQ (SEQ ID NO: 113); KYQVIV (SEQ ID NO: 114); KYVIVQ (SEQ ID NO: 115); VVYIQK (SEQ ID NO: 116); IVQVYK (SEQ ID NO: 117); KQIVYV (SEQ ID NO: 118); KYVIQV (SEQ ID NO: 119); QKVIVY (SEQ ID NO: 120); QYIKW (SEQ ID NO: 121); QYIVKV (SEQ ID NO: 122); VQIVYK (SEQ ID NO: 123); WQKYI (SEQ ID NO: 124); VYQIVK (SEQ ID NO: 125); YVQVIK (SEQ ID NO: 126); QIVYVK (SEQ ID NO: 127); IKVYQV (SEQ ID NO: 128); KVVYIQ (SEQ ID NO: 129); VQKYIV (SEQ ID NO: 130); VQKYVI (SEQ ID NO: 131); KWQIY (SEQ ID NO: 132); YVIKIY (SEQ ID NO: 133); QVIVYV (SEQ ID NO: 134); WIKVY (SEQ ID NO: 135); KWIYI (SEQ ID NO: 136); VYWVVIK (SEQ ID NO: 137); YIVQVY (SEQ ID NO: 138); YVIQVY (SEQ ID NO: 139); YVIWY (SEQ ID NO: 140); KVIVYI (SEQ ID NO: 141); YVIKVY (SEQ ID NO: 142); VQVIVK (SEQ ID NO: 143); YVVKIY (SEQ ID NO: 144); YWQVI (SEQ ID NO: 145); QIVVYQ (SEQ ID NO: 146); WQIVK (SEQ ID NO: 147); VQIWK (SEQ ID NO: 148); VIVVYK (SEQ ID NO: 149); IVQVYI (SEQ ID NO: 150); YVVIQV (SEQ ID NO: 151); VYQWI (SEQ ID NO: 152); KYIQVY (SEQ ID NO: 153); KYWIY (SEQ ID NO: 154); KYVQIY (SEQ ID NO: 155); WQVIK (SEQ ID NO: 156); QKIWK (SEQ ID NO: 157); KYVIVY (SEQ ID NO: 158); KQWIK (SEQ ID NO: 159); YVQIYV (SEQ ID NO: 160); YKVQVY (SEQ ID NO: 161); IKVQIY (SEQ ID NO: 162); KIWQK (SEQ ID NO: 163); VIKVVI (SEQ ID NO: 164); VYIKVV (SEQ ID NO: 165); VYIVQV (SEQ ID NO: 166); IVYVQI (SEQ ID NO: 167); KVQIYK (SEQ ID NO: 168); IYVIVY (SEQ ID NO: 169); VIYVIV (SEQ ID NO: 170); KYQVYI (SEQ ID NO: 171); VIQKW (SEQ ID NO: 172); VKIVYV (SEQ ID NO: 173); VKQIW (SEQ ID NO: 174); YIVKQY (SEQ ID NO: 175); YIWQY (SEQ ID NO: 176); YKIQVY (SEQ ID NO: 177); YQWIY (SEQ ID NO: 178); YVVKQY (SEQ ID NO: 179); VIVKVQ (SEQ ID NO: 180); QKWIK (SEQ ID NO: 181); GMVVVG (SEQ ID NO: 182); GVWMG (SEQ ID NO: 183); GVVMVG (SEQ ID NO: 184); GGVWM (SEQ ID NO: 185); GWGVM (SEQ ID NO: 186); GVVVGM (SEQ ID NO: 187); GVMWG (SEQ ID NO: 188); GGVVMV (SEQ ID NO: 189); GGVMW (SEQ ID NO: 190); GMWGV (SEQ ID NO: 191); GVVMGV (SEQ ID NO: 192); GMVGW (SEQ ID NO: 193); GVMVGV (SEQ ID NO: 194); GWGMV (SEQ ID NO: 195); GGMVW (SEQ ID NO: 196); GVGVMV (SEQ ID NO: 197); GVGWM (SEQ ID NO: 198); MVVVGM (SEQ ID NO: 199); GMGVVV (SEQ ID NO: 200); MVGG (SEQ ID NO: 201); GVGMW (SEQ ID NO: 202); GWGGV (SEQ ID NO: 203); MVGWG (SEQ ID NO: 204); VVMVGG (SEQ ID NO: 205); MGVVVWG (SEQ ID NO: 206); MVGVGV (SEQ ID NO: 207); VGVMVG (SEQ ID NO: 208); MWGVG (SEQ ID NO: 209); WGVMG (SEQ ID NO: 210); VMGWG (SEQ ID NO: 211); VGVGVM (SEQ ID NO: 212); VMVGVG (SEQ ID NO: 213); MGGVVV (SEQ ID NO: 214); VGMWG (SEQ ID NO: 215); WGVGM (SEQ ID NO: 216); VGGWM (SEQ ID NO: 217); WGMVG (SEQ ID NO: 218); VGWMG (SEQ ID NO: 219); MVGGW (SEQ ID NO: 220); VGMVGV (SEQ ID NO: 221); VMWGG (SEQ ID NO: 222); WGGVM (SEQ ID NO: 223); VGGVMV (SEQ ID NO: 224); VWVGMG (SEQ ID NO: 225); WGMGV (SEQ ID NO: 226); MGVGW (SEQ ID NO: 227); VGWGM (SEQ ID NO: 228); VGGMW (SEQ ID NO: 229); VWMGG (SEQ ID NO: 230); WMGGV (SEQ ID NO: 231); VWGGM (SEQ ID NO: 232); WGGMV (SEQ ID NO: 233); VGMGW (SEQ ID NO: 234); VMGGW (SEQ ID NO: 235); VGGVGV (SEQ ID NO: 236); VGVMGV (SEQ ID NO: 237); VGVGMV (SEQ ID NO: 238); and VMVGGV (SEQ ID NO: 239); ANSTSV (SEQ ID NO: 240); ANSVSG (SEQ ID NO: 241); ANSVSS (SEQ ID NO: 242); AQNSNV (SEQ ID NO: 243); AQNVNS (SEQ ID NO: 244); AQNVTS (SEQ ID NO: 245); AQSQSV (SEQ ID NO: 246); AQSSSV (SEQ ID NO: 247); AQSTSV (SEQ ID NO: 248); AQSVNS (SEQ ID NO: 249); AQSVQS (SEQ ID NO: 250); AQSVSQ (SEQ ID NO: 251); AQSVSS (SEQ ID NO: 252); AQSVST (SEQ ID NO: 253); ASNNNV (SEQ ID NO: 254); ASNQNQ (SEQ ID NO: 255); ASNQNV (SEQ ID NO: 256); ASNQTQ (SEQ ID NO: 257); ASNSNV (SEQ ID NO: 258); ASNSTV (SEQ ID NO: 259); ASNTNS (SEQ ID NO: 260); ASNTNV (SEQ ID NO: 261); ASNTSV (SEQ ID NO: 262); ASNVNG (SEQ ID NO: 263); ASNVNQ (SEQ ID NO: 264); ASNVNS (SEQ ID NO: 265); ASNVNT (SEQ ID NO: 266); ASNVTG (SEQ ID NO: 267); ASNVTT (SEQ ID NO: 268); ASSNSV (SEQ ID NO: 269); ASSVSG (SEQ ID NO: 270); ASSVSN (SEQ ID NO: 271); ATNVNS (SEQ ID NO: 272); ATNVTS (SEQ ID NO: 273); ATSQSQ (SEQ ID NO: 274); ATSQSV (SEQ ID NO: 275); ATSTSG (SEQ ID NO: 276); ATSTSV (SEQ ID NO: 277); ATSVSG (SEQ ID NO: 278); ATSVSS (SEQ ID NO: 279); AVNQNS (SEQ ID NO: 280); AVNQSQ (SEQ ID NO: 281); AVNSNG (SEQ ID NO: 282); AVNSNS (SEQ ID NO: 283); AVNSNT (SEQ ID NO: 284); AVNTNS (SEQ ID NO: 285); AVSNSG (SEQ ID NO: 286); AVSNSS (SEQ ID NO: 287); AVSQNQ (SEQ ID NO: 288); AVSQSG (SEQ ID NO: 289); AVSQSQ (SEQ ID NO: 290); AVSQTQ (SEQ ID NO: 291); AVSSNQ (SEQ ID NO: 292); AVSSNS (SEQ ID NO: 293); AVSSSQ (SEQ ID NO: 294); AVSTSG (SEQ ID NO: 295); GANTVS (SEQ ID NO: 296); GAQTSS (SEQ ID NO: 297); GASNQS (SEQ ID NO: 298); GASQQS (SEQ ID NO: 299); GASSQQ (SEQ ID NO: 300) GGQVTS (SEQ ID NO: 301); GGSNQV (SEQ ID NO: 302); GNNVQS (SEQ ID NO: 303); GNQVTS (SEQ ID NO: 304); GNSNQV (SEQ ID NO: 305); GNSQQQ (SEQ ID NO: 306); GNSQQS (SEQ ID NO: 307); GNSQQV (SEQ ID NO: 308); GNSSTV (SEQ ID NO: 309); GNSTQS (SEQ ID NO: 310); GNSTQV (SEQ ID NO: 311); GNSTVS (SEQ ID NO: 312); GNSVQS (SEQ ID NO: 313); GNSVSS (SEQ ID NO: 314); GNSVST (SEQ ID NO: 315); GNSVTS (SEQ ID NO: 316); GQNTVS (SEQ ID NO: 317); GQNVAS (SEQ ID NO: 318); GQNVQS (SEQ ID NO: 319); GQNVSS (SEQ ID NO: 320); GQNVTS (SEQ ID NO: 321); GQQQSQ (SEQ ID NO: 322); GQQTSS (SEQ ID NO: 323); GQQTSV (SEQ ID NO: 324); GQQVAS (SEQ ID NO: 325); GQQVNS (SEQ ID NO: 326); GQQVQS (SEQ ID NO: 327); GQQVSG (SEQ ID NO: 328); GQQVSQ (SEQ ID NO: 329); GQQVSS (SEQ ID NO: 330); GQQVST (SEQ ID NO: 331); GQQVTS (SEQ ID NO: 332); GQSGQV (SEQ ID NO: 333); GQSNQA (SEQ ID NO: 334); GQSNQS (SEQ ID NO: 335); GQSNQV (SEQ ID NO: 336); GQSQAQ (SEQ ID NO: 337); GQSQQQ (SEQ ID NO: 338); GQSQQS (SEQ ID NO: 339); GQSQSQ (SEQ ID NO: 340); GQSSQQ (SEQ ID NO: 341); GQSSQS (SEQ ID NO: 342); GQSSQV (SEQ ID NO: 343); GQSTQS (SEQ ID NO: 344); GQSTQV (SEQ ID NO: 345); GQSVAG (SEQ ID NO: 346); GQSVAQ (SEQ ID NO: 347); GQSVAS (SEQ ID NO: 348); GQSVQN (SEQ ID NO: 349); GQSVQQ (SEQ ID NO: 350); GQSVQS (SEQ ID NO: 351); GQSVSG (SEQ ID NO: 352); GQSVSN (SEQ ID NO: 353); GQSVSQ (SEQ ID NO: 354); GQSVSS (SEQ ID NO: 355); GQSVST (SEQ ID NO: 356); GQSVTS (SEQ ID NO: 357); GSNQQV (SEQ ID NO: 358); GSNQVQ (SEQ ID NO: 359); GSNSTV (SEQ ID NO: 360); GSNSVQ (SEQ ID NO: 361); GSNSVT (SEQ ID NO: 362); GSNTAV (SEQ ID NO: 363); GSNTQV (SEQ ID NO: 364); GSNTVA (SEQ ID NO: 365); GSNTVS (SEQ ID NO: 366); GSNVAS (SEQ ID NO: 367); GSNVQQ (SEQ ID NO: 368); GSNVQS (SEQ ID NO: 369); GSNVQT (SEQ ID NO: 370); GSNVTS (SEQ ID NO: 371); GSQQSV (SEQ ID NO: 372); GSQQTQ (SEQ ID NO: 373); GSQQTV (SEQ ID NO: 374); GSQTSS (SEQ ID NO: 375); GSQTSV (SEQ ID NO: 376); GSQVAS (SEQ ID NO: 377); GSQVNS (SEQ ID NO: 378); GSQVQS (SEQ ID NO: 379); GSQVSS (SEQ ID NO: 380); GSQVST (SEQ ID NO: 381); GSQVTG (SEQ ID NO: 382); GSQVTS (SEQ ID NO: 383); GSSAQS (SEQ ID NO: 384); GSSGQV (SEQ ID NO: 385); GSSNQA (SEQ ID NO: 386); GSSNQT (SEQ ID NO: 387); GSSNQV (SEQ ID NO: 388); GSSNSV (SEQ ID NO: 389); GSSNTV (SEQ ID NO: 390); GSSNVT (SEQ ID NO: 391); GSSQAQ (SEQ ID NO: 392); GSSQQA (SEQ ID NO: 393); GSSQQV (SEQ ID NO: 394); GSSQSQ (SEQ ID NO: 395); GSSSQQ (SEQ ID NO: 396); GSSSQS (SEQ ID NO: 397); GSSSQV (SEQ ID NO: 398); GSSTQA (SEQ ID NO: 399); GSSTQG (SEQ ID NO: 400); GSSTQT (SEQ ID NO: 401); GSSTQV (SEQ ID NO: 402); GSSTSV (SEQ ID NO: 403); GSSTVN (SEQ ID NO: 404); GSSVNS (SEQ ID NO: 405); GSSVQN (SEQ ID NO: 406); GSSVQT (SEQ ID NO: 407); GSSVSG (SEQ ID NO: 408); GSSVSS (SEQ ID NO: 409); GSSVST (SEQ ID NO: 410); GSSVTN (SEQ ID NO: 411); GTNSSV (SEQ ID NO: 412); GTNSVS (SEQ ID NO: 413); GTNTVS (SEQ ID NO: 414); GTNVQS (SEQ ID NO: 415); GTNVSS (SEQ ID NO: 416); GTNVTS (SEQ ID NO: 417); GTQQSQ (SEQ ID NO: 418); GTQSTS (SEQ ID NO: 419); GTQSTV (SEQ ID NO: 420); GTQTSA (SEQ ID NO: 421); GTQTSV (SEQ ID NO: 422); GTQVQS (SEQ ID NO: 423); GTQVSG (SEQ ID NO: 424); GTQVSN (SEQ ID NO: 425); GTQVSS (SEQ ID NO: 426); GTQVST (SEQ ID NO: 427); GTSAQS (SEQ ID NO: 428); GTSGQV (SEQ ID NO: 429); GTSNQA (SEQ ID NO: 430); GTSNQS (SEQ ID NO: 431); GTSNQT (SEQ ID NO: 432); GTSNQV (SEQ ID NO: 433); GTSNSV (SEQ ID NO: 434); GTSNVS (SEQ ID NO: 435); GTSQQA (SEQ ID NO: 436); GTSQQS (SEQ ID NO: 437); GTSQQV (SEQ ID NO: 438); GTSQSQ (SEQ ID NO: 439); GTSQSV (SEQ ID NO: 440); GTSSNV (SEQ ID NO: 441); GTSTQA (SEQ ID NO: 442); GTSTQS (SEQ ID NO: 443); GTSTQT (SEQ ID NO: 444); GTSTQV (SEQ ID NO: 445); GTSTSV (SEQ ID NO: 446); GTSVAG (SEQ ID NO: 447); GTSVAS (SEQ ID NO: 448); GTSVNS (SEQ ID NO: 449); GTSVNT (SEQ ID NO: 450); GTSVQG (SEQ ID NO: 451); GTSVQN (SEQ ID NO: 452); GTSVQQ (SEQ ID NO: 453); GTSVQS (SEQ ID NO: 454); GTSVQT (SEQ ID NO: 455); GTSVSG (SEQ ID NO: 456); GTSVSN (SEQ ID NO: 457); GTSVSS (SEQ ID NO: 458); GTSVST (SEQ ID NO: 459); GTSVTS (SEQ ID NO: 460); GVNSQS (SEQ ID NO: 461); GVNSST (SEQ ID NO: 462); GVNSTS (SEQ ID NO: 463); GVNTQS (SEQ ID NO: 464); GVNTSS (SEQ ID NO: 465); GVQNTS (SEQ ID NO: 466); GVQQSQ (SEQ ID NO: 467); GVQSNQ (SEQ ID NO: 468); GVQSNS (SEQ ID NO: 469); GVQSSG (SEQ ID NO: 470); GVQSSQ (SEQ ID NO: 471); GVQSTS (SEQ ID NO: 472); GVQSTT (SEQ ID NO: 473); GVQTNS (SEQ ID NO: 474); GVQTQS (SEQ ID NO: 475); GVQTSG (SEQ ID NO: 476); GVQTSS (SEQ ID NO: 477); GVSGQG (SEQ ID NO: 478); GVSGQT (SEQ ID NO: 479); GVSNAS (SEQ ID NO: 480); GVSNNV (SEQ ID NO: 481); GVSNQG (SEQ ID NO: 482); GVSNQN (SEQ ID NO: 483); GVSNQQ (SEQ ID NO: 484); GVSNQS (SEQ ID NO: 485); GVSNQT (SEQ ID NO: 486); GVSNSS (SEQ ID NO: 487); GVSNST (SEQ ID NO: 488); GVSNTS (SEQ ID NO: 489); GVSQAQ (SEQ ID NO: 490); GVSQNQ (SEQ ID NO: 491); GVSQNS (SEQ ID NO: 492); GVSQQG (SEQ ID NO: 493); GVSQQS (SEQ ID NO: 494); GVSQQT (SEQ ID NO: 495); GVSQSG (SEQ ID NO: 496); GVSQSQ (SEQ ID NO: 497); GVSQSS (SEQ ID NO: 498); GVSSAQ (SEQ ID NO: 499); GVSSAS (SEQ ID NO: 500); GVSSGQ (SEQ ID NO: 501); GVSSNG (SEQ ID NO: 502); GVSSNQ (SEQ ID NO: 503); GVSSNS (SEQ ID NO: 504); GVSSNT (SEQ ID NO: 505); GVSSQG (SEQ ID NO: 506); GVSSQN (SEQ ID NO: 507); GVSSQQ (SEQ ID NO: 508); GVSSSG (SEQ ID NO: 509); GVSSSQ (SEQ ID NO: 510); GVSSSS (SEQ ID NO: 511); GVSSTN (SEQ ID NO: 512); GVSTAS (SEQ ID NO: 513); GVSTNS (SEQ ID NO: 514); GVSTQG (SEQ ID NO: 515); GVSTQN (SEQ ID NO: 516); GVSTQQ (SEQ ID NO: 517); GVSTQS (SEQ ID NO: 518); GVSTQT (SEQ ID NO: 519); GVSTSN (SEQ ID NO: 520); GVSTSQ (SEQ ID NO: 521); GVSTSS (SEQ ID NO: 522); GVSTST (SEQ ID NO: 523); GVTNSS (SEQ ID NO: 524); GVTSNS (SEQ ID NO: 525); GVTSSN (SEQ ID NO: 526); NGSTSV (SEQ ID NO: 527); NGSVTS (SEQ ID NO: 528); NNSVSS (SEQ ID NO: 529); NQSQSQ (SEQ ID NO: 530); NQSVSN (SEQ ID NO: 531); NQSVSQ (SEQ ID NO: 532); NQSVSS (SEQ ID NO: 533); NSSNSV (SEQ ID NO: 534); NSSQSQ (SEQ ID NO: 535); NSSTVG (SEQ ID NO: 536); NSSVSG (SEQ ID NO: 537); NSSVSN (SEQ ID NO: 538); NSSVSS (SEQ ID NO: 539); NSSVTG (SEQ ID NO: 540); NTNVNS (SEQ ID NO: 541); NTQVSS (SEQ ID NO: 542); NTSGSV (SEQ ID NO: 543); NTSQSQ (SEQ ID NO: 544); NTSTSS (SEQ ID NO: 545); NTSTSV (SEQ ID NO: 546); NTSVSS (SEQ ID NO: 547); NVSGST (SEQ ID NO: 548); NVSNSG (SEQ ID NO: 549); NVSQSG (SEQ ID NO: 550); NVSQSQ (SEQ ID NO: 551); NVSQSS (SEQ ID NO: 552); NVSSSG (SEQ ID NO: 553); NVSSSS (SEQ ID NO: 554); NVSSTG (SEQ ID NO: 555); NVSTSN (SEQ ID NO: 556); NVSTSS (SEQ ID NO: 557); NVSTST (SEQ ID NO: 558); QNSTSV (SEQ ID NO: 559); QNSVSG (SEQ ID NO: 560); QQSQSQ (SEQ ID NO: 561); QQSVAG (SEQ ID NO: 562); QQSVAQ (SEQ ID NO: 563); QQSVAS (SEQ ID NO: 564); QQSVAT (SEQ ID NO: 565); QQSVSG (SEQ ID NO: 566); QQSVSN (SEQ ID NO: 567); QQSVSS (SEQ ID NO: 568); QSNQVQ (SEQ ID NO: 569); QSNTAV (SEQ ID NO: 570); QSNTNV (SEQ ID NO: 571); QSSNSV (SEQ ID NO: 572); QSSQAQ (SEQ ID NO: 573); QSSQSQ (SEQ ID NO: 574); QSSQSV (SEQ ID NO: 575); QSSTSV (SEQ ID NO: 576); QSSVSG (SEQ ID NO: 577); QSSVSN (SEQ ID NO: 578); QSSVSQ (SEQ ID NO: 579); QSSVSS (SEQ ID NO: 580); QSSVTG (SEQ ID NO: 581); QSSVTS (SEQ ID NO: 582); QTSQSQ (SEQ ID NO: 583); QTSSSQ (SEQ ID NO: 584); QTSSSV (SEQ ID NO: 585); QTSVAG (SEQ ID NO: 586); QTSVAS (SEQ ID NO: 587); QTSVSG (SEQ ID NO: 588); QTSVSN (SEQ ID NO: 589); QTSVSS (SEQ ID NO: 590); QTSVST (SEQ ID NO: 591); QTSVTS (SEQ ID NO: 592); QVSNSG (SEQ ID NO: 593); QVSNSS (SEQ ID NO: 594); QVSNST (SEQ ID NO: 595); QVSQAQ (SEQ ID NO: 596); QVSQTG (SEQ ID NO: 597); QVSQTQ (SEQ ID NO: 598); QVSSAQ (SEQ ID NO: 599); QVSSSG (SEQ ID NO: 600); QVSSSQ (SEQ ID NO: 601); QVSSSS (SEQ ID NO: 602); QVSSTQ (SEQ ID NO: 603); QVSTSG (SEQ ID NO: 604); QVSTSN (SEQ ID NO: 605); QVSTSS (SEQ ID NO: 606); QVSTST (SEQ ID NO: 607); SAQQSQ (SEQ ID NO: 608); SAQQTQ (SEQ ID NO: 609); SASQQQ (SEQ ID NO: 610); SASQSQ (SEQ ID NO: 611); SGNSTV (SEQ ID NO: 612); SGNTSV (SEQ ID NO: 613); SGNVST (SEQ ID NO: 614); SGNVTS (SEQ ID NO: 615); SGQQTQ (SEQ ID NO: 616); SGQQTV (SEQ ID NO: 617); SGQTSV (SEQ ID NO: 618); SGQVSS (SEQ ID NO: 619); SGQVTG (SEQ ID NO: 620); SGQVTQ (SEQ ID NO: 621); SGQVTT (SEQ ID NO: 622); SGSNTV (SEQ ID NO: 623); SGSTNV (SEQ ID NO: 624); SGSTQV (SEQ ID NO: 625); SGSVAS (SEQ ID NO: 626); SGSVNT (SEQ ID NO: 627); SGSVQG (SEQ ID NO: 628); SGSVQS (SEQ ID NO: 629); SNNQTV (SEQ ID NO: 630); SNNTAV (SEQ ID NO: 631); SNNTNV (SEQ ID NO: 632); SNNTQV (SEQ ID NO: 633); SNQQSQ (SEQ ID NO: 634); SNQQSV (SEQ ID NO: 635); SNQQTQ (SEQ ID NO: 636); SNQQTV (SEQ ID NO: 637); SNQSTV (SEQ ID NO: 638); SNQTSV (SEQ ID NO: 639); SNQVSG (SEQ ID NO: 640); SNQVSQ (SEQ ID NO: 641); SNQVSS (SEQ ID NO: 642); SNQVST (SEQ ID NO: 643); SNQVTG (SEQ ID NO: 644); SNQVTQ (SEQ ID NO: 645); SNQVTS (SEQ ID NO: 646); SNSGAV (SEQ ID NO: 647); SNSNAV (SEQ ID NO: 648); SNSNGV (SEQ ID NO: 649); SNSNQS (SEQ ID NO: 650); SNSQAG (SEQ ID NO: 651); SNSQAQ (SEQ ID NO: 652); SNSQAS (SEQ ID NO: 653); SNSQAT (SEQ ID NO: 654); SNSQAV (SEQ ID NO: 655); SNSQGQ (SEQ ID NO: 656); SNSQGV (SEQ ID NO: 657); SNSQQA (SEQ ID NO: 658); SNSQQG (SEQ ID NO: 659); SNSQQQ (SEQ ID NO: 660); SNSQQS (SEQ ID NO: 661); SNSQQT (SEQ ID NO: 662); SNSQQV (SEQ ID NO: 663); SNSQSG (SEQ ID NO: 664); SNSQST (SEQ ID NO: 665); SNSQSV (SEQ ID NO: 666); SNSSAV (SEQ ID NO: 667); SNSSGV (SEQ ID NO: 668); SNSSQG (SEQ ID NO: 669); SNSSQQ (SEQ ID NO: 670); SNSSQT (SEQ ID NO: 671); SNSSQV (SEQ ID NO: 672); SNSSSQ (SEQ ID NO: 673); SNSSSV (SEQ ID NO: 674); SNSTAG (SEQ ID NO: 675); SNSTGS (SEQ ID NO: 676); SNSTNV (SEQ ID NO: 677); SNSTQA (SEQ ID NO: 678); SNSTQQ (SEQ ID NO: 679); SNSTQS (SEQ ID NO: 680); SNSTQV (SEQ ID NO: 681); SNSTSA (SEQ ID NO: 682); SNSTSV (SEQ ID NO: 683); SNSTVG (SEQ ID NO: 684); SNSVAQ (SEQ ID NO: 685); SNSVAS (SEQ ID NO: 686); SNSVAT (SEQ ID NO: 687); SNSVGQ (SEQ ID NO: 688); SNSVGS (SEQ ID NO: 689); SNSVGT (SEQ ID NO: 690); SNSVNS (SEQ ID NO: 691); SNSVNT (SEQ ID NO: 692); SNSVQQ (SEQ ID NO: 693); SNSVQS (SEQ ID NO: 694); SNSVSG (SEQ ID NO: 695); SNSVSQ (SEQ ID NO: 696); SNSVSS (SEQ ID NO: 697); SNSVST (SEQ ID NO: 698); SNSVTG (SEQ ID NO: 699); SQNVAS (SEQ ID NO: 700); SQNVNS (SEQ ID NO: 701); SQQQSQ (SEQ ID NO: 702); SQQQSV (SEQ ID NO: 703); SQQQTQ (SEQ ID NO: 704); SQQQTV (SEQ ID NO: 705); SQQSTV (SEQ ID NO: 706); SQQVSG (SEQ ID NO: 707); SQQVSN (SEQ ID NO: 708); SQQVSS (SEQ ID NO: 709); SQQVST (SEQ ID NO: 710); SQQVTG (SEQ ID NO: 711); SQQVTN (SEQ ID NO: 712); SQQVTS (SEQ ID NO: 713); SQQVTT (SEQ ID NO: 714); SQSNAS (SEQ ID NO: 715); SQSNAV (SEQ ID NO: 716); SQSNGV (SEQ ID NO: 717); SQSNQT (SEQ ID NO: 718); SQSNQV (SEQ ID NO: 719); SQSNSV (SEQ ID NO: 720); SQSQAQ (SEQ ID NO: 721); SQSQAS (SEQ ID NO: 722); SQSQAV (SEQ ID NO: 723); SQSQQQ (SEQ ID NO: 724); SQSQQV (SEQ ID NO: 725); SQSQSQ (SEQ ID NO: 726); SQSSAV (SEQ ID NO: 727); SQSSGV (SEQ ID NO: 728); SQSSQG (SEQ ID NO: 729); SQSSQV (SEQ ID NO: 730); SQSSSQ (SEQ ID NO: 731); SQSSSV (SEQ ID NO: 732); SQSTAV (SEQ ID NO: 733); SQSTQS (SEQ ID NO: 734); SQSTSG (SEQ ID NO: 735); SQSTSV (SEQ ID NO: 736); SQSVAG (SEQ ID NO: 737); SQSVAN (SEQ ID NO: 738); SQSVAQ (SEQ ID NO: 739); SQSVAS (SEQ ID NO: 740); SQSVAT (SEQ ID NO: 741); SQSVGG (SEQ ID NO: 742); SQSVGN (SEQ ID NO: 743); SQSVGQ (SEQ ID NO: 744); SQSVGS (SEQ ID NO: 745); SQSVGT (SEQ ID NO: 746); SQSVNG (SEQ ID NO: 747); SQSVNS (SEQ ID NO: 748); SQSVQG (SEQ ID NO: 749); SQSVQN (SEQ ID NO: 750); SQSVQQ (SEQ ID NO: 751); SQSVQS (SEQ ID NO: 752); SQSVQT (SEQ ID NO: 753); SQSVSG (SEQ ID NO: 754); SQSVSN (SEQ ID NO: 755); SQSVSS (SEQ ID NO: 756); SQSVST (SEQ ID NO: 757); SSNGTV (SEQ ID NO: 758); SSNQNQ (SEQ ID NO: 759); SSNQNV (SEQ ID NO: 760); SSNQTQ (SEQ ID NO: 761); SSNQTV (SEQ ID NO: 762); SSNSTV (SEQ ID NO: 763); SSNTNV (SEQ ID NO: 764); SSNTQV (SEQ ID NO: 765); SSNTVG (SEQ ID NO: 766); SSNVAQ (SEQ ID NO: 767); SSNVAS (SEQ ID NO: 768); SSNVGT (SEQ ID NO: 769); SSNVNG (SEQ ID NO: 770); SSNVNQ (SEQ ID NO: 771); SSNVNS (SEQ ID NO: 772); SSNVNT (SEQ ID NO: 773); SSNVQS (SEQ ID NO: 774); SSNVQT (SEQ ID NO: 775); SSNVTG (SEQ ID NO: 776); SSNVTQ (SEQ ID NO: 777); SSNVTS (SEQ ID NO: 778); SSQNTV (SEQ ID NO: 779); SSQQSV (SEQ ID NO: 780); SSQQTQ (SEQ ID NO: 781); SSQQTV (SEQ ID NO: 782); SSQSTQ (SEQ ID NO: 783); SSQTSV (SEQ ID NO: 784); SSQVNS (SEQ ID NO: 785); SSQVSN (SEQ ID NO: 786); SSQVSQ (SEQ ID NO: 787); SSQVSS (SEQ ID NO: 788); SSQVST (SEQ ID NO: 789); SSQVTG (SEQ ID NO: 790); SSQVTQ (SEQ ID NO: 791); SSQVTS (SEQ ID NO: 792); SSQVTT (SEQ ID NO: 793); SSSNAV (SEQ ID NO: 794); SSSNQV (SEQ ID NO: 795); SSSNSV (SEQ ID NO: 796); SSSQAQ (SEQ ID NO: 797); SSSQNQ (SEQ ID NO: 798); SSSQQQ (SEQ ID NO: 799); SSSQQT (SEQ ID NO: 800); SSSQQV (SEQ ID NO: 801); SSSQSQ (SEQ ID NO: 802); SSSQSV (SEQ ID NO: 803); SSSSGQ (SEQ ID NO: 804); SSSSGV (SEQ ID NO: 805); SSSSNV (SEQ ID NO: 806); SSSSQQ (SEQ ID NO: 807); SSSSQS (SEQ ID NO: 808); SSSTAV (SEQ ID NO: 809); SSSTNV (SEQ ID NO: 810); SSSTSV (SEQ ID NO: 811); SSSVAQ (SEQ ID NO: 812); SSSVAS (SEQ ID NO: 813); SSSVGG (SEQ ID NO: 814); SSSVGQ (SEQ ID NO: 815); SSSVGS (SEQ ID NO: 816); SSSVNG (SEQ ID NO: 817); SSSVNS (SEQ ID NO: 818); SSSVNT (SEQ ID NO: 819); SSSVQG (SEQ ID NO: 820); SSSVQN (SEQ ID NO: 821); SSSVQS (SEQ ID NO: 822); SSSVQT (SEQ ID NO: 823); SSSVSG (SEQ ID NO: 824); SSSVSN (SEQ ID NO: 825); SSSVSQ (SEQ ID NO: 826); SSSVSS (SEQ ID NO: 827); SSSVST (SEQ ID NO: 828); STNGSV (SEQ ID NO: 829); STNSGV (SEQ ID NO: 830); STNSNV (SEQ ID NO: 831); STNTNV (SEQ ID NO: 832); STNTQV (SEQ ID NO: 833); STNVGS (SEQ ID NO: 834); STNVNG (SEQ ID NO: 835); STNVNS (SEQ ID NO: 836); STNVSG (SEQ ID NO: 837); STNVSS (SEQ ID NO: 838); STNVTG (SEQ ID NO: 839); STNVTS (SEQ ID NO: 840); STQQSG (SEQ ID NO: 841); STQQSQ (SEQ ID NO: 842); STQQSV (SEQ ID NO: 843); STQQTQ (SEQ ID NO: 844); STQQTS (SEQ ID NO: 845); STQQTV (SEQ ID NO: 846); STQSNV (SEQ ID NO: 847); STQSTV (SEQ ID NO: 848); STQTSS (SEQ ID NO: 849); STQTSV (SEQ ID NO: 850); STQVNS (SEQ ID NO: 851); STQVSG (SEQ ID NO: 852); STQVSN (SEQ ID NO: 853); STQVST (SEQ ID NO: 854); STQVTG (SEQ ID NO: 855); STQVTN (SEQ ID NO: 856); STQVTQ (SEQ ID NO: 857); STQVTS (SEQ ID NO: 858); STQVTT (SEQ ID NO: 859); STSGNV (SEQ ID NO: 860); STSNAS (SEQ ID NO: 861); STSNGV (SEQ ID NO: 862); STSNQV (SEQ ID NO: 863); STSNSV (SEQ ID NO: 864); STSQAQ (SEQ ID NO: 865); STSQGV (SEQ ID NO: 866); STSQNQ (SEQ ID NO: 867); STSQQQ (SEQ ID NO: 868); STSQQV (SEQ ID NO: 869); STSQSQ (SEQ ID NO: 870); STSSAV (SEQ ID NO: 871); STSSGV (SEQ ID NO: 872); STSSQQ (SEQ ID NO: 873); STSSQS (SEQ ID NO: 874); STSSQV (SEQ ID NO: 875); STSSSQ (SEQ ID NO: 876); STSTAV (SEQ ID NO: 877); STSTGV (SEQ ID NO: 878); STSTQA (SEQ ID NO: 879); STSTQG (SEQ ID NO: 880); STSTQT (SEQ ID NO: 881); STSTQV (SEQ ID NO: 882); STSTSQ (SEQ ID NO: 883); STSTSV (SEQ ID NO: 884); STSVAG (SEQ ID NO: 885); STSVAN (SEQ ID NO: 886); STSVAQ (SEQ ID NO: 887); STSVAS (SEQ ID NO: 888); STSVAT (SEQ ID NO: 889); STSVGG (SEQ ID NO: 890); STSVGN (SEQ ID NO: 891); STSVGQ (SEQ ID NO: 892); STSVGS (SEQ ID NO: 893); STSVNG (SEQ ID NO: 894); STSVNN (SEQ ID NO: 895); STSVNS (SEQ ID NO: 896); STSVQG (SEQ ID NO: 897); STSVQN (SEQ ID NO: 898); STSVQQ (SEQ ID NO: 899); STSVQS (SEQ ID NO: 900); STSVQT (SEQ ID NO: 901); STSVSG (SEQ ID NO: 902); STSVSN (SEQ ID NO: 903); STSVSQ (SEQ ID NO: 904); STSVSS (SEQ ID NO: 905); STSVST (SEQ ID NO: 906); SVNGST (SEQ ID NO: 907); SVNGTS (SEQ ID NO: 908); SVNQAQ (SEQ ID NO: 909); SVNQQQ (SEQ ID NO: 910); SVNSGT (SEQ ID NO: 911); SVNSNQ (SEQ ID NO: 912); SVNSNS (SEQ ID NO: 913); SVNSTG (SEQ ID NO: 914); SVNSTS (SEQ ID NO: 915); SVNTGS (SEQ ID NO: 916); SVNTSG (SEQ ID NO: 917); SVQQSQ (SEQ ID NO: 918); SVQQST (SEQ ID NO: 919); SVQQTQ (SEQ ID NO: 920); SVQQTS (SEQ ID NO: 921); SVQQTT (SEQ ID NO: 922); SVQSNQ (SEQ ID NO: 923); SVQSNS (SEQ ID NO: 924); SVQSSG (SEQ ID NO: 925); SVQSSQ (SEQ ID NO: 926); SVQSSS (SEQ ID NO: 927); SVQSTG (SEQ ID NO: 928); SVQSTQ (SEQ ID NO: 929); SVQSTS (SEQ ID NO: 930); SVQTSG (SEQ ID NO: 931); SVQTSN (SEQ ID NO: 932); SVQTSS (SEQ ID NO: 933); SVQVSN (SEQ ID NO: 934); SVSGNT (SEQ ID NO: 935); SVSGQS (SEQ ID NO: 936); SVSNAQ (SEQ ID NO: 937); SVSNAS (SEQ ID NO: 938); SVSNGS (SEQ ID NO: 939); SVSNGT (SEQ ID NO: 940); SVSNQG (SEQ ID NO: 941); SVSNQS (SEQ ID NO: 942); SVSNQT (SEQ ID NO: 943); SVSNST (SEQ ID NO: 944); SVSNTG (SEQ ID NO: 945); SVSQAQ (SEQ ID NO: 946); SVSQAS (SEQ ID NO: 947); SVSQGQ (SEQ ID NO: 948); SVSQNQ (SEQ ID NO: 949); SVSQQG (SEQ ID NO: 950); SVSQQQ (SEQ ID NO: 951); SVSQQS (SEQ ID NO: 952); SVSQSG (SEQ ID NO: 953); SVSQSS (SEQ ID NO: 954); SVSQTQ (SEQ ID NO: 955); SVSSAQ (SEQ ID NO: 956); SVSSAS (SEQ ID NO: 957); SVSSGG (SEQ ID NO: 958); SVSSGS (SEQ ID NO: 959); SVSSGT (SEQ ID NO: 960); SVSSNG (SEQ ID NO: 961); SVSSNQ (SEQ ID NO: 962); SVSSNS (SEQ ID NO: 963); SVSSNT (SEQ ID NO: 964); SVSSQG (SEQ ID NO: 965); SVSSQQ (SEQ ID NO: 966); SVSSQS (SEQ ID NO: 967); SVSSQT (SEQ ID NO: 968); SVSSQV (SEQ ID NO: 969); SVSSSG (SEQ ID NO: 970); SVSSSQ (SEQ ID NO: 971); SVSSSS (SEQ ID NO: 972); SVSTAG (SEQ ID NO: 973); SVSTAS (SEQ ID NO: 974); SVSTAT (SEQ ID NO: 975); SVSTGS (SEQ ID NO: 976); SVSTNS (SEQ ID NO: 977); SVSTNT (SEQ ID NO: 978); SVSTQQ (SEQ ID NO: 979); SVSTQS (SEQ ID NO: 980); SVSTQT (SEQ ID NO: 981); SVSTSG (SEQ ID NO: 982); SVSTSN (SEQ ID NO: 983); SVSTSS (SEQ ID NO: 984); SVSTST (SEQ ID NO: 985); TASQSQ (SEQ ID NO: 986); TASQVQ (SEQ ID NO: 987); TGSNSV (SEQ ID NO: 988); TGSNVS (SEQ ID NO: 989); TGSSNV (SEQ ID NO: 990); TGSVNS (SEQ ID NO: 991); TGSVSN (SEQ ID NO: 992); TNSGSV (SEQ ID NO: 993); TNSGVS (SEQ ID NO: 994); TNSQVQ (SEQ ID NO: 995); TNSSGV (SEQ ID NO: 996); TNSVGS (SEQ ID NO: 997); TNSVSG (SEQ ID NO: 998); TQSQSQ (SEQ ID NO: 999); TQSQVQ (SEQ ID NO: 1000); TQSTVS (SEQ ID NO: 1001); TQSVSN (SEQ ID NO: 1002); TQSVSS (SEQ ID NO: 1003); TSNGVS (SEQ ID NO: 1004); TSNVGS (SEQ ID NO: 1005); TSNVSG (SEQ ID NO: 1006); TSSGNV (SEQ ID NO: 1007); TSSGVN (SEQ ID NO: 1008); TSSNGV (SEQ ID NO: 1009); TSSNSV (SEQ ID NO: 1010); TSSNVG (SEQ ID NO: 1011); TSSQSQ (SEQ ID NO: 1012); TSSSVQ (SEQ ID NO: 1013); TSSVGN (SEQ ID NO: 1014); TSSVNG (SEQ ID NO: 1015); TSSVSG (SEQ ID NO: 1016); TSSVSS (SEQ ID NO: 1017); TSSVTS (SEQ ID NO: 1018); TVNSGS (SEQ ID NO: 1019); TVNSSG (SEQ ID NO: 1020); TVSGNS (SEQ ID NO: 1021); TVSGSN (SEQ ID NO: 1022); TVSNGS (SEQ ID NO: 1023); TVSNSG (SEQ ID NO: 1024); TVSNST (SEQ ID NO: 1025); TVSNVG (SEQ ID NO: 1026); TVSQNQ (SEQ ID NO: 1027); TVSQQV (SEQ ID NO: 1028); TVSQSG (SEQ ID NO: 1029); TVSQSQ (SEQ ID NO: 1030); TVSSGN (SEQ ID NO: 1031); TVSSNG (SEQ ID NO: 1032); TVSSNQ (SEQ ID NO: 1033); TVSSNS (SEQ ID NO: 1034); TVSSSQ (SEQ ID NO: 1035); TVSTSG (SEQ ID NO: 1036); TVSTSN (SEQ ID NO: 1037); TVSTSS (SEQ ID NO: 1038); TVSTST (SEQ ID NO: 1039); VGSTNS (SEQ ID NO: 1040); VNSTSG (SEQ ID NO: 1041); VNSTSN (SEQ ID NO: 1042); VSSQSQ (SEQ ID NO: 1043); VSSQVQ (SEQ ID NO: 1044); VSSTNG (SEQ ID NO: 1045); VTSNSG (SEQ ID NO: 1046); VTSQSQ (SEQ ID NO: 1047), SVNDLV (SEQ ID NO: 1059); LKVKVL (SEQ ID NO: 1060), NKGAII (SEQ ID NO: 1061), and NVSTSG (SEQ ID NO: 1062).

In particular embodiments, the peptide includes one or more of the following hexapeptides, where each indicated amino acid is either an L-amino acid or a D-amino acid: SVNLDV (SEQ ID NO: 5); VEALYL (SEQ ID NO: 1048); LYQLEN (SEQ ID NO: 1049); VQIVYK (SEQ ID NO: 123); GYVIIK (SEQ ID NO: 1050); SNQNNF (SEQ ID NO: 1051); SSQVTQ (SEQ ID NO: 791); SVLTSL (SEQ ID NO: 1052); SSTNVG (SEQ ID NO: 1053); SVSSSY (SEQ ID NO: 1054); GAILSS (SEQ ID NO: 1055); GAIIGL (SEQ ID NO: 1056); AIIGLM (SEQ ID NO: 1057); MVGGW (SEQ ID NO: 220); GGWIA (SEQ ID NO: 1058).

Variants of peptides disclosed herein can also be used, so long as the variant continues to contribute to fibril formation. “Variants” include protein sequences having one or more amino acid additions, deletions, stop positions, or substitutions, as compared to a protein sequence disclosed elsewhere herein.

An amino acid substitution can be a conservative or a non-conservative substitution. Variants of protein sequence disclosed herein can include those having one or more conservative amino acid substitutions. A “conservative substitution” or “conservative amino acid substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, or sulfur-containing). For example, an aliphatic grouping may include, for purposes of substitution, G, A, V, L, and I. Other groups including amino acids that are considered conservative substitutions for one another include: sulfur-containing: M and C; acidic: D, E, N, and Q; small aliphatic, nonpolar or slightly polar residues: A, S, T, P, and G; polar, negatively charged residues and their amides: D, N, E, and Q; polar, positively charged residues: H, R, and K; large aliphatic, nonpolar residues: M, L, I, V, and C; and large aromatic residues: F, Y, and W.

Non-conservative substitutions include those that significantly affect: the structure of the peptide backbone in the area of the alteration (e.g., the alpha-helical or beta-sheet structure); the charge or hydrophobicity of the molecule at the target site; or the bulk of the side chain. Non-conservative substitutions which in general are expected to produce the greatest changes in the protein's properties are those in which (i) a hydrophilic residue (e.g. S or T) can be substituted for (or by) a hydrophobic residue (e.g. L, I, F, V, or A); (ii) a C or P can be substituted for (or by) any other residue; (iii) a residue having an electropositive side chain (e.g. K, R, or H) can be substituted for (or by) an electronegative residue (e.g. Q or D); or (iv) a residue having a bulky side chain (e.g. F), can be substituted for (or by) one not having a bulky side chain, (e.g. G). Additional information is found in Creighton (1984) Proteins, W.H. Freeman and Company.

Variants of fibril-forming peptides disclosed herein include proteins that share: 70% sequence identity with any of SEQ ID NOs: 1-1064; 75% sequence identity with any of SEQ ID NOs: 1-1064; 80% sequence identity with any of SEQ ID NOs: 1-1064; 81% sequence identity with any of SEQ ID NOs: 1-1064; 82% sequence identity with any of 1-1064; 83% sequence identity with any of SEQ ID NOs: 1-1064; 84% sequence identity with any of SEQ ID NOs: 1-1064; 85% sequence identity with any of SEQ ID NOs: 1-1064; 86% sequence identity with any of SEQ ID NOs: 1-1064; 87% sequence identity with any of SEQ ID NOs: 1-1064; 88% sequence identity with any of SEQ ID NOs: 1-1064; 89% sequence identity with any of SEQ ID NOs: 1-1064; 90% sequence identity with any of SEQ ID NOs: 1-1064; 91% sequence identity with any of SEQ ID NOs: 1-1064; 92% sequence identity with any of SEQ ID NOs: 1-1064; 93% sequence identity with any of SEQ ID NOs: 1-1064; 94% sequence identity with any of SEQ ID NOs: 1-1064; 95% sequence identity with any of SEQ ID NOs: 1-1064; 96% sequence identity with any of SEQ ID NOs: 1-1064; 97% sequence identity with any of SEQ ID NOs: 1-1064; 98% sequence identity with any of SEQ ID NOs: 1-1064; or 99% sequence identity with any of SEQ ID NOs: 1-1064.

“Percent (%) amino acid sequence identity” with respect to the sequences identified herein is defined as the percentage of amino acid residues in a candidate sequence that are identical with the amino acid residues in the reference sequence after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, % amino acid sequence identity values generated using the WU-BLAST-2 computer program (Altschul et al., Methods in Enzymology, 266:460-480 (1996)) uses several search parameters, most of which are set to the default values. Those that are not set to default values (i.e., the adjustable parameters) are set with the following values: overlap span=1, overlap fraction=0.125, word threshold (T)=11 and scoring matrix BLOSUM62.

In particular embodiments, fibrils formed from the peptides disclosed herein are capable of forming a complex with one or more plasma proteins. A “complex” refers to a molecular entity formed by non-covalent association involving two or more component molecular entities (ionic or uncharged). The bonding between the components is normally weaker than in a covalent bond. Typically, the dissociation constant for a complex (“K_{d}”) is equal to or greater than 1 μM.

Nonlimiting examples of such plasma proteins include (symbol for plasma protein indicated in parenthesis after the plasma protein): Apolipoprotein B-100 (A); Complement C3 (B); Complement C1s (C); Beta-2-glycoprotein 1 (D); Clusterin (E); Coagulation Factor V (F); Complement C1r (G); Apolipoprotein A-I (H); ITIH2 (I); Complement 1qB (J); Apolipoprotein A-IV (K); Complement Factor H (L); Fibrinogen beta chain (M); Complement C4-A (N); Transthyretin (0); SerpinG1 Plasma protease C1 inhibitor (P); Fibrinogen alpha chain (Q); Complement C1qA (R); Vitronectin (S); Serpina-1 Alpha-1-antitrypsin (T); VitaminD-binding protein (U); Haptoglobin-related protein (V); ITIH4 (W); Fibrinogen gamma chain (X); SerpinC1 Antithrombin-III (Y); Apolipoprotein A-2 (Z); Complement Factor H-related protein (AA); Gelsolin (BB); Complement factor B (CC); Alpha-2-HS-glycoprotein (DD); Serum paraoxinase/arylesterase 1 (EE); Complement C5 (FF); Apolipoprotein C5 (GG); Apolipoprotein C-II (HH); Apolipoprotein C-I (II); ITIH1 (JJ); von Willebrand factor (KK); Ceruloplasmin (LL); Apolipoprotein E (MM); Filamin-A (NN); Histidine-rich glycoprotein (00); SerpinF2 alpha-2-antiplasmin (PP); Coagulation factor II Prothrombin (QQ); Coagulation factor X (RR); Vitamin K-dependent protein (SS); Apolipoprotein C-III (TT); Alpha-I-acid glycoprotein 2 (UU); Coagulation factor IX (VV); Apolipoprotein M (WW); Serum Albumin (XX).

Examples of peptide fibril/plasma protein complexes that are capable of being formed include (symbol for hexamer followed by symbol for plasma protein): 1B, 2B, 3B, 4B, 5B, 6B, 7B, 8B, 9B, 10B, 11B, 12B, 13B, 14B, 15B, 16B, 17B, 18B, 19B, 20B, 21B, 22B, 23B, 24B, 25B, 26B, 27B, 28B, 29B, 30B, 31B, 32B, 33B, 34B, 35B, 36B, 37B, 38B, 39B, 40B, 41B, 42B, 43B, 44B, 45B, 46B, 47B, 48B, 49B, 50B, 51B, 52B, 53B, 54B, 55B, 56B, 57B, 58B, 59B, 60B, 61B, 62B, 63B, 64B, 65B, 66B, 67B, 68B, 69B, 70B, 71B, 72B, 73B, 74B, 75B, 76B, 77B, 78B, 79B, 80B, 81B, 82B, 83B, 84B, 85B, 86B, 87B, 88B, 89B, 90B, 91B, 92B, 93B, 94B, 95B, 96B, 97B, 98B, 99B, 100B, 101B, 102B, 103B, 104B, 105B, 106B, 107B, 108B, 109B, 110B, 111B, 112B, 113B, 114B, 115B, 116B, 117B, 118B, 119B, 120B, 121B, 122B, 123B, 124B, 125B, 126B, 127B, 128B, 129B, 130B, 131B, 132B, 133B, 134B, 135B, 136B, 137B, 138B, 139B, 140B, 141B, 142B, 143B, 144B, 145B, 146B, 147B, 148B, 149B, 150B, 151B, 152B, 153B, 154B, 155B, 156B, 157B, 158B, 159B, 160B, 161B, 162B, 163B, 164B, 165B, 166B, 167B, 168B, 169B, 170B, 171B, 172B, 173B, 174B, 175B, 176B, 177B, 178B, 179B, 180B, 181B, 182B, 183B, 184B, 185B, 186B, 187B, 188B, 189B, 190B, 191B, 192B, 193B, 194B, 195B, 196B, 197B, 198B, 199B, 200B, 201B, 202B, 203B, 204B, 205B, 206B, 207B, 208B, 209B, 210B, 211B, 212B, 213B, 214B, 215B, 216B, 217B, 218B, 219B, 220B, 221B, 222B, 223B, 224B, 225B, 226B, 227B, 228B, 229B, 230B, 231B, 232B, 233B, 234B, 235B, 236B, 237B, 238B, 239B, 240B, 241B, 242B, 243B, 244B, 245B, 246B; and 247B-1047B; 1C, 2C, 3C, 4C, 5C, 6C, 7C, 8C, 9C, 10C, 11C, 12C, 13C, 14C, 15C, 16C, 17C, 18C, 19C, 20C, 21C, 22C, 23C, 24C, 25C, 26C, 27C, 28C, 29C, 30C, 31C, 32C, 33C, 34C, 35C, 36C, 37C, 38C, 39C, 40C, 41C, 42C, 43C, 44C, 45C, 46C, 47C, 48C, 49C, 50C, 51C, 52C, 53C, 54C, 55C, 56C, 57C, 58C, 59C, 60C, 61C, 62C, 63C, 64C, 65C, 66C, 67C, 68C, 69C, 70C, 71C, 72C, 73C, 74C, 75C, 76C, 77C, 78C, 79C, 80C, 81C, 82C, 83C, 84C, 85C, 86C, 87C, 88C, 89C, 90C, 91C, 92C, 93C, 94C, 95C, 96C, 97C, 98C, 99C, 100C, 101C, 102C, 103C, 104C, 105C, 106C, 107C, 108C, 109C, 110C, 111C, 112C, 113C, 114C, 115C, 116C, 117C, 118C, 119C, 120C, 121C, 122C, 123C, 124C, 125C, 126C, 127C, 128C, 129C, 130C, 131C, 132C, 133C, 134C, 135C, 136C, 137C, 138C, 139C, 140C, 141C, 142C, 143C, 144C, 145C, 146C, 147C, 148C, 149C, 150C, 151C, 152C, 153C, 154C, 155C, 156C, 157C, 158C, 159C, 160C, 161C, 162C, 163C, 164C, 165C, 166C, 167C, 168C, 169C, 170C, 171C, 172C, 173C, 174C, 175C, 176C, 177C, 178C, 179C, 180C, 181C, 182C, 183C, 184C, 185C, 186C, 187C, 188C, 189C, 190C, 191C, 192C, 193C, 194C, 195C, 196C, 197C, 198C, 199C, 200C, 201C, 202C, 203C, 204C, 205C, 206C, 207C, 208C, 209C, 210C, 211C, 212C, 213C, 214C, 215C, 216C, 217C, 218C, 219C, 220C, 221C, 222C, 223C, 224C, 225C, 226C, 227C, 228C, 229C, 230C, 231C, 232C, 233C, 234C, 235C, 236C, 237C, 238C, 239C, 240C, 241C, 242C, 243C, 244C, 245C, 246C; and 247C-1047C; 1F, 2F, 3F, 4F, 5F, 6F, 7F, 8F, 9F, 10F, 11F, 12F, 13F, 14F, 15F, 16F, 17F, 18F, 19F, 20F, 21F, 22F, 23F, 24F, 25F, 26F, 27F, 28F, 29F, 30F, 31F, 32F, 33F, 34F, 35F, 36F, 37F, 38F, 39F, 40F, 41F, 42F, 43F, 44F, 45F, 46F, 47F, 48F, 49F, 50F, 51F, 52F, 53F, 54F, 55F, 56F, 57F, 58F, 59F, 60F, 61F, 62F, 63F, 64F, 65F, 66F, 67F, 68F, 69F, 70F, 71F, 72F, 73F, 74F, 75F, 76F, 77F, 78F, 79F, 80F, 81F, 82F, 83F, 84F, 85F, 86F, 87F, 88F, 89F, 90F, 91F, 92F, 93F, 94F, 95F, 96F, 97F, 98F, 99F, 100F, 101F, 102F, 103F, 104F, 105F, 106F, 107F, 108F, 109F, 110F, 111F, 112F, 113F, 114F, 115F, 116F, 117F, 118F, 119F, 120F, 121F, 122F, 123F, 124F, 125F, 126F, 127F, 128F, 129F, 130F, 131F, 132F, 133F, 134F, 135F, 136F, 137F, 138F, 139F, 140F, 141F, 142F, 143F, 144F, 145F, 146F, 147F, 148F, 149F, 150F, 151F, 152F, 153F, 154F, 155F, 156F, 157F, 158F, 159F, 160F, 161F, 162F, 163F, 164F, 165F, 166F, 167F, 168F, 169F, 170F, 171F, 172F, 173F, 174F, 175F, 176F, 177F, 178F, 179F, 180F, 181F, 182F, 183F, 184F, 185F, 186F, 187F, 188F, 189F, 190F, 191F, 192F, 193F, 194F, 195F, 196F, 197F, 198F, 199F, 200F, 201F, 202F, 203F, 204F, 205F, 206F, 207F, 208F, 209F, 210F, 211F, 212F, 213F, 214F, 215F, 216F, 217F, 218F, 219F, 220F, 221F, 222F, 223F, 224F, 225F, 226F, 227F, 228F, 229F, 230F, 231F, 232F, 233F, 234F, 235F, 236F, 237F, 238F, 239F, 240F, 241F, 242F, 243F, 244F, 245F, 246F; and 247F-1047F; 1G, 2G, 3G, 4G, 5G, 6G, 7G, 8G, 9G, 10G, 11G, 12G, 13G, 14G, 15G, 16G, 17G, 18G, 19G, 20G, 21 G, 22G, 23G, 24G, 25G, 26G, 27G, 28G, 29G, 30G, 31G, 32G, 33G, 34G, 35G, 36G, 37G, 38G, 39G, 40G, 41G, 42G, 43G, 44G, 45G, 46G, 47G, 48G, 49G, 50G, 51G, 52G, 53G, 54G, 55G, 56G, 57G, 58G, 59G, 60G, 61G, 62G, 63G, 64G, 65G, 66G, 67G, 68G, 69G, 70G, 71G, 72G, 73G, 74G, 75G, 76G, 77G, 78G, 79G, 80G, 81G, 82G, 83G, 84G, 85G, 86G, 87G, 88G, 89G, 90G, 91G, 92G, 93G, 94G, 95G, 96G, 97G, 98G, 99G, 100G, 101G, 102G, 103G, 104G, 105G, 106G, 107G, 108G, 1090, 110G, 1110, 112G, 113G, 114G, 115G, 116G, 117G, 118G, 119G, 120G, 121G, 122G, 123G, 124G, 125G, 126G, 127G, 128G, 129G, 130G, 131G, 1320, 133G, 134G, 135G, 136G, 137G, 138G, 139G, 140G, 141G, 142G, 143G, 144G, 145G, 146G, 147G, 148G, 149G, 150G, 151G, 152G, 153G, 154G, 155G, 156G, 157G, 158G, 159G, 160G, 161G, 162G, 163G, 164G, 165G, 166G, 167G, 168G, 169G, 170G, 171G, 172G, 173G, 1740, 175G, 176G, 177G, 178G, 179G, 180G, 181G, 182G, 183G, 184G, 185G, 186G, 187G, 188G, 189G, 190G, 191G, 192G, 193G, 194G, 195G, 196G, 197G, 198G, 199G, 200G, 201G, 202G, 203G, 204G, 205G, 206G, 207G, 208G, 209G, 210G, 211 G, 212G, 213G, 214G, 215G, 216G, 217G, 218G, 219G, 220G, 221 G, 222G, 223G, 224G, 225G, 226G, 227G, 228G, 229G, 230G, 231 G, 232G, 233G, 234G, 235G, 236G, 237G, 238G, 239G, 240G, 241G, 242G, 243G, 244G, 245G, 246G; and 247G-1047G; IM, 2M, 3M, 4M, 5M, 6M, 7M, 8M, 9M, 10M, 11M, 12M, 13M, 14M, 15M, 16M, 17M, IBM, 19M, 20M, 21 M, 22M, 23M, 24M, 25M, 26M, 27M, 28M, 29M, 30M, 31 M, 32M, 33M, 34M, 35M, 36M, 37M, 38M, 39M, 40M, 41M, 42M, 43M, 44M, 45M, 46M, 47M, 48M, 49M, 50M, 51M, 52M, 53M, 54M, 55M, 56M, 57M, 58M, 59M, 60M, 61M, 62M, 63M, 64M, 65M, 66M, 67M, 68M, 69M, 70M, 71M, 72M, 73M, 74M, 75M, 76M, 77M, 78M, 79M, B0M, BIM, 82M, 83M, 84M, 85M, 86M, 87M, 88M, 89M, 90M, 91M, 92M, 93M, 94M, 95M, 96M, 97M, 98M, 99M, 100M, 101M, 102M, 103M, 104M, 105M, 106M, 107M, 108M, 109M, 110M, 111M, 112M, 113M, 114M, 115M, 116M, 117M, 118M, 119M, 120M, 121 M, 122M, 123M, 124M, 125M, 126M, 127M, 128M, 129M, 130M, 131 M, 132M, 133M, 134M, 135M, 136M, 137M, 138M, 139M, 140M, 141 M, 142M, 143M, 144M, 145M, 146M, 147M, 148M, 149M, 150M, 151 M, 152M, 153M, 154M, 155M, 156M, 157M, 158M, 159M, 160M, 161 M, 162M, 163M, 164M, 165M, 166M, 167M, 168M, 169M, 170M, 171 M, 172M, 173M, 174M, 175M, 176M, 177M, 178M, 179M, 180M, 181 M, 182M, 183M, 184M, 185M, 186M, 187M, 188M, 189M, 190M, 191 M, 192M, 193M, 194M, 195M, 196M, 197M, 198M, 199M, 200M, 201 M, 202M, 203M, 204M, 205M, 206M, 207M, 208M, 209M, 210M, 211 M, 212M, 213M, 214M, 215M, 216M, 217M, 218M, 219M, 220M, 221 M, 222M, 223M, 224M, 225M, 226M, 227M, 228M, 229M, 230M, 231 M, 232M, 233M, 234M, 235M, 236M, 237M, 238M, 239M, 240M, 241 M, 242M, 243M, 244M, 245M, 246M; and 247M-1047M; 1J, 2J, 3J, 4J, 5J, 6J, 7.1, 8J, 9J, 10J, 11J, 12J, 13J, 14J, 15J, 16J, 17J, 18J, 19J, 20J, 21J, 22J, 23J, 24.1, 25J, 26J, 27J, 28J, 29J, 30J, 31J, 32J, 33J, 34J, 35J, 36J, 37J, 38J, 39J, 40J, 41J, 42J, 43J, 44J, 45J, 46J, 47J, 48.1, 49J, 50J, 51J, 52J, 53J, 54J, 55J, 56J, 57J, 58J, 59J, 60J, 61J, 62J, 63J, 64J, 65J, 66J, 67J, 68J, 69J, 70J, 71.1, 72J, 73.1, 74J, 75J, 76J, 77J, 78J, 79J, 80J, 81J, 82J, 83J, 84J, 85J, 86J, 87J, 88J, 89J, 9W, 91J, 92J, 93J, 94J, 95J, 96J, 97J, 98J, 99J, 100J, 101J, 102J, 103J, 104J, 105J, 106J, 107J, 108J, 109J, 110J, 111J, 112J, 113J, 114J, 115J, 116J, 117J, 118J, 119J, 120J, 121J, 122J, 123J, 124.1, 125J, 126J, 127J, 128J, 129J, 130J, 131J, 132J, 133J, 134J, 135J, 136J, 137J, 138J, 139J, 140J, 141J, 142J, 143J, 144J, 145J, 146J, 147J, 148J, 149J, 150J, 151J, 152J, 153J, 154J, 155J, 156J, 157J, 158J, 159J, 160J, 161J, 162J, 163J, 164J, 165J, 166J, 167J, 168J, 169J, 170J, 171J, 172J, 173J, 174J, 175J, 176J, 177J, 178J, 179J, 180J, 181J, 182J, 183.1, 184J, 185J, 186.1, 187J, 188J, 189.1, 190J, 191J, 192J, 193J, 194J, 195J, 196J, 197J, 198J, 199J, 200J, 201J, 202J, 203J, 204J, 205J, 206J, 207J, 208J, 209J, 21W, 211J, 212J, 213J, 214J, 215J, 216J, 217J, 218J, 219J, 220J, 221J, 222J, 223J, 224J, 225J, 22W, 227J, 228J, 229J, 230J, 231J, 232J, 233J, 234.1, 235J, 236J, 237J, 238J, 239J, 240J, 241J, 242J, 243J, 244J, 245J, 246J; and 247J-1047J; IL, 2L, 3L, 4L, 5L, 6L, 7L, 8L, 9L, 10L, 11L, 12L, 13L, 14L, 15L, 16L, 17L, 18L, 19L, 20L, 21L, 22L, 23L, 24L, 25L, 26L, 27L, 28L, 29L, 30L, 31L, 32L, 33L, 34L, 35L, 36L, 37L, 38L, 39L, 40L, 41L, 42L, 43L, 44L, 45L, 46L, 47L, 48L, 49L, 50L, 51L, 52L, 53L, 54L, 55L, 56L, 57L, 58L, 59L, 60L, 61L, 62L, 63L, 64L, 65L, 66L, 67L, 68L, 69L, 70L, 71L, 72L, 73L, 74L, 75L, 76L, 77L, 78L, 79L, 80L, 81L, 82L, 83L, 84L, 85L, 86L, 87L, 88L, 89L, 90L, 91L, 92L, 93L, 94L, 95L, 96L, 97L, 98L, 99L, 100L, 101L, 102L, 103L, 104L, 105L, 106L, 107L, 108L, 109L, 110L, 111L, 112L, 113L, 114L, 115L, 116L, 117L, 118L, 119L, 120L, 121L, 122L, 123L, 124L, 125L, 126L, 127L, 128L, 129L, 130L, 131L, 132L, 133L, 134L, 135L, 136L, 137L, 138L, 139L, 140L, 141L, 142L, 143L, 144L, 145L, 146L, 147L, 148L, 149L, 150L, 151L, 152L, 153L, 154L, 155L, 156L, 157L, 158L, 159L, 160L, 161L, 162L, 163L, 164L, 165L, 166L, 167L, 168L, 169L, 170L, 171L, 172L, 173L, 174L, 175L, 176L, 177L, 178L, 179L, 180L, 181L, 182L, 183L, 184L, 185L, 186L, 187L, 188L, 189L, 190L, 191L, 192L, 193L, 194L, 195L, 196L, 197L, 198L, 199L, 200L, 201L, 202L, 203L, 204L, 205L, 206L, 207L, 208L, 209L, 210L, 211L, 212L, 213L, 214L, 215L, 216L, 217L, 218L, 219L, 220L, 221L, 222L, 223L, 224L, 225L, 226L, 227L, 228L, 229L, 230L, 231L, 232L, 233L, 234L, 235L, 236L, 237L, 238L, 239L, 240L, 241L, 242L, 243L, 244L, 245L, 246L; and 247L-1047L; 1V, 2V, 3V, 4V, 5V, 6V, 7V, 8V, 9V, 10V, 11V, 12V, 13V, 14V, 15V, 16V, 17V, 18V, 19V, 20V, 21V, 22V, 23V, 24V, 25V, 26V, 27V, 28V, 29V, 30V, 31V, 32V, 33V, 34V, 35V, 36V, 37V, 38V, 39V, 40V, 41V, 42V, 43V, 44V, 45V, 46V, 47V, 48V, 49V, 50V, 51V, 52V, 53V, 54V, 55V, 56V, 57V, 58V, 59V, 60V, 61V, 62V, 63V, 64V, 65V, 66V, 67V, 68V, 69V, 70V, 71V, 72V, 73V, 74V, 75V, 76V, 77V, 78V, 79V, 80V, 81V, 82V, 83V, 84V, 85V, 86V, 87V, 88V, 89V, 90V, 91V, 92V, 93V, 94V, 95V, 96V, 97V, 98V, 99V, 100V, 101V, 102V, 103V, 104V, 105V, 106V, 107V, 108V, 109V, 110V, 111V, 112V, 113V, 114V, 115V, 116V, 117V, 118V, 119V, 120V, 121V, 122V, 123V, 124V, 125V, 126V, 127V, 128V, 129V, 130V, 131V, 132V, 133V, 134V, 135V, 136V, 137V, 138V, 139V, 140V, 141V, 142V, 143V, 144V, 145V, 146V, 147V, 148V, 149V, 150V, 151V, 152V, 153V, 154V, 155V, 156V, 157V, 158V, 159V, 160V, 161V, 162V, 163V, 164V, 165V, 166V, 167V, 168V, 169V, 170V, 171V, 172V, 173V, 174V, 175V, 176V, 177V, 178V, 179V, 180V, 181V, 182V, 183V, 184V, 185V, 186V, 187V, 188V, 189V, 190V, 191V, 192V, 193V, 194V, 195V, 196V, 197V, 198V, 199V, 200V, 201V, 202V, 203V, 204V, 205V, 206V, 207V, 208V, 209V, 210V, 21 IV, 212V, 213V, 214V, 215V, 216V, 217V, 218V, 219V, 220V, 221V, 222V, 223V, 224V, 225V, 226V, 227V, 228V, 229V, 230V, 231V, 232V, 233V, 234V, 235V, 236V, 237V, 238V, 239V, 240V, 241V, 242V, 243V, 244V, 245V, 246V; and 247V-1047V; 1KK, 2KK, 3KK, 4KK, 5KK, 6KK, 7KK, 8KK, 9KK, 10KK, 11KK, 12KK, 13KK, 14KK, 15KK, 16KK, 17KK, 18KK, 19KK, 20KK, 21KK, 22KK, 23KK, 24KK, 25KK, 26KK, 27KK, 28KK, 29KK, 30KK, 31 KK, 32KK, 33KK, 34KK, 35KK, 36KK, 37KK, 38KK, 39KK, 40KK, 41KK, 42KK, 43KK, 44KK, 45KK, 46KK, 47KK, 48KK, 49KK, 50KK, 51KK, 52KK, 53KK, 54KK, 55KK, 56KK, 57KK, 58KK, 59KK, 60KK, 61KK, 62KK, 63KK, 64KK, 65KK, 66KK, 67KK, 68KK, 69KK, 70KK, 71 KK, 72KK, 73KK, 74KK, 75KK, 76KK, 77KK, 78KK, 79KK, 80KK, 81KK, 82KK, 83KK, 84KK, 85KK, 86KK, 87KK, 88KK, 89KK, 90KK, 91KK, 92KK, 93KK, 94KK, 95KK, 96KK, 97KK, 98KK, 99KK, 100KK, 101 KK, 102KK, 103KK, 104KK, 105KK, 106KK, 107KK, 108KK, 109KK, 110KK, 111KK, 112KK, 113KK, 114KK, 115KK, 116KK, 117KK, 118KK, 119KK, 120KK, 121KK, 122KK, 123KK, 124KK, 125KK, 126KK, 127KK, 128KK, 129KK, 130KK, 131KK, 132KK, 133KK, 134KK, 135KK, 136KK, 137KK, 138KK, 139KK, 140KK, 141KK, 142KK, 143KK, 144KK, 145KK, 146KK, 147KK, 148KK, 149KK, 150KK, 151KK, 152KK, 153KK, 154KK, 155KK, 156KK, 157KK, 158KK, 159KK, 160KK, 161KK, 162KK, 163KK, 164KK, 165KK, 166KK, 167KK, 168KK, 169KK, 170KK, 171KK, 172KK, 173KK, 174KK, 175KK, 176KK, 177KK, 178KK, 179KK, 180KK, 181KK, 182KK, 183KK, 184KK, 185KK, 186KK, 187KK, 188KK, 189KK, 190KK, 191KK, 192KK, 193KK, 194KK, 195KK, 196KK, 197KK, 198KK, 199KK, 200KK, 201KK, 202KK, 203KK, 204KK, 205KK, 206KK, 207KK, 208KK, 209KK, 210KK, 211KK, 212KK, 213KK, 214KK, 215KK, 216KK, 217KK, 218KK, 219KK, 220KK, 221KK, 222KK, 223KK, 224KK, 225KK, 226KK, 227KK, 228KK, 229KK, 230KK, 231KK, 232KK, 233KK, 234KK, 235KK, 236KK, 237KK, 238KK, 239KK, 240KK, 241KK, 242KK, 243KK, 244KK, 245KK, 246KK; and 247KK-1047KK; 1LL, 2LL, 3LL, 4LL, 5LL, 6LL, 7LL, 8LL, 9LL, 10LL, 11LL, 12LL, 13LL, 14LL, 15LL, 16LL, 17LL, 18LL, 19LL, 20LL, 21LL, 22LL, 23LL, 24LL, 25LL, 26LL, 27LL, 28LL, 29LL, 30LL, 31LL, 32LL, 33LL, 34LL, 35LL, 36LL, 37LL, 38LL, 39LL, 40LL, 41LL, 42LL, 43LL, 44LL, 45LL, 46LL, 47LL, 48LL, 49LL, 50LL, 51LL, 52LL, 53LL, 54LL, 55LL, 56LL, 57LL, 58LL, 59LL, 60LL, 61LL, 62LL, 63LL, 64LL, 65LL, 66LL, 67LL, 68LL, 69LL, TOLL, 71LL, 72LL, 73LL, 74LL, 75LL, 76LL, 77LL, 78LL, 79LL, 80LL, 81LL, 82LL, 83LL, 84LL, 85LL, 86LL, 87LL, 88LL, 89LL, 90LL, 91LL, 92LL, 93LL, 94LL, 95LL, 96LL, 97LL, 98LL, 99LL, 100LL, 101LL, 102LL, 103LL, 104LL, 105LL, 106LL, 107LL, 108LL, 109LL, 110LL, 111LL, 112LL, 113LL, 114LL, 115LL, 116LL, 117LL, 118LL, 119LL, 120LL, 121LL, 122LL, 123LL, 124LL, 125LL, 126LL, 127LL, 128LL, 129LL, 130LL, 131LL, 132LL, 133LL, 134LL, 135LL, 136LL, 137LL, 138LL, 139LL, 140LL, 141LL, 142LL, 143LL, 144LL, 145LL, 146LL, 147LL, 148LL, 149LL, 150LL, 151LL, 152LL, 153LL, 154LL, 155LL, 156LL, 157LL, 158LL, 159LL, 160LL, 161LL, 162LL, 163LL, 164LL, 165LL, 166LL, 167LL, 168LL, 169LL, 170LL, 171LL, 172LL, 173LL, 174LL, 175LL, 176LL, 177LL, 178LL, 179LL, 180LL, 181LL, 182LL, 183LL, 184LL, 185LL, 186LL, 187LL, 188LL, 189LL, 190LL, 191LL, 192LL, 193LL, 194LL, 195LL, 196LL, 197LL, 198LL, 199LL, 200LL, 201LL, 202LL, 203LL, 204LL, 205LL, 206LL, 207LL, 208LL, 209LL, 210LL, 211LL, 212LL, 213LL, 214LL, 215LL, 216LL, 217LL, 218LL, 219LL, 220LL, 221LL, 222LL, 223LL, 224LL, 225LL, 226LL, 227LL, 228LL, 229LL, 230LL, 231LL, 232LL, 233LL, 234LL, 235LL, 236LL, 237LL, 238LL, 239LL, 240LL, 241LL, 242LL, 243LL, 244LL, 245LL, 246LL; and 247LL-1047LL; 1XX, 2XX, 3XX, 4XX, 5XX, 6XX, 7XX, 8XX, 9XX, 10XX, 11XX, 12XX, 13XX, 14XX, 15XX, 16XX, 17XX, 18XX, 19XX, 20XX, 21XX, 22XX, 23XX, 24XX, 25XX, 26XX, 27XX, 28XX, 29XX, 30XX, 31XX, 32XX, 33XX, 34XX, 35XX, 36XX, 37XX, 38XX, 39XX, 40XX, 41XX, 42XX, 43XX, 44XX, 45XX, 46XX, 47XX, 48XX, 49XX, 50XX, 51XX, 52XX, 53XX, 54XX, 55XX, 56XX, 57XX, 58XX, 59XX, 60XX, 61XX, 62XX, 63XX, 64XX, 65XX, 66XX, 67XX, 68XX, 69XX, 70XX, 71XX, 72XX, 73XX, 74XX, 75XX, 76XX, 77XX, 78XX, 79XX, 80XX, 81XX, 82XX, 83XX, 84XX, 85XX, 86XX, 87XX, 88XX, 89XX, 90XX, 91XX, 92XX, 93XX, 94XX, 95XX, 96XX, 97XX, 98XX, 99XX, 100XX, 101XX 102XX, 103XX, 104XX, 105XX, 106XX, 107XX, 108XX, 1509XX, 110XX, 111XX, 112XX, 113XX, 114XX, 115XX, 116XX, 117XX, 118XX, 119XX, 120XX, 121XX, 122XX, 123XX, 124XX, 125XX, 126XX, 127XX, 128XX, 129XX, 130XX, 131XX, 132XX, 133XX, 134XX, 135XX, 136XX, 137XX, 138XX, 139XX, 140XX, 141XX, 142XX, 144XX, 145XX, 146XX, 147XX, 148XX, 149XX, 150XX, 151XX, 152XX, 153XX, 154XX, 155XX, 156XX, 157XX, 158XX, 159XX, 160XX, 161XX, 162XX, 163XX, 164XX, 165XX, 166XX, 167XX, 168XX, 169XX, 170XX, 171XX, 172XX, 173XX, 174XX, 175XX, 176XX, 177XX, 178XX, 179XX, 180XX, 181XX, 182XX, 183XX, 184XX, 185XX, 186XX, 187XX, 188XX, 189XX, 190XX, 191XX, 192XX, 193XX, 194XX, 195XX, 196XX, 197XX, 198XX, 199XX, 200XX, 201XX, 202XX, 203XX, 204XX, 205XX, 206XX, 207XX, 208XX, 209XX, 210XX, 211XX, 212XX, 213XX, 214XX, 215XX, 216XX, 217XX, 218XX, 219XX, 220XX, 221XX, 222XX, 223XX, 224XX, 225XX, 226XX, 227XX, 228XX, 229XX, 230XX, 231XX, 232XX, 233XX, 234XX, 235XX, 236XX, 237XX, 238XX, 239XX, 240XX, 241XX, 242XX, 243XX, 244XX, 245XX, 246XX; and 247XX-1047XX.

As indicated, these peptides can be formulated for administration to the respiratory tract. Therapeutically effective amounts (also referred to herein as doses) that lead to effective treatments can be initially estimated based on results from in vitro assays and/or animal model studies. Such information can be used to more accurately determine useful doses in subjects of interest.

The actual dose amount administered to a particular subject can be determined by a physician, veterinarian, or researcher taking into account parameters such as physical, physiological and psychological factors including target, body weight, type of condition, stage or severity of condition, previous or concurrent therapeutic interventions, idiopathy of the subject, etc. Exemplary doses can include, for example, 0.1 μg/kg-1000 mg/kg.

### EXEMPLARY EMBODIMENTS

1. A method of targeting and activating B-1a lymphocytes in the pleural cavity to treat a medical condition including an inflammatory or autoimmune component in a subject in need thereof including: administering a therapeutically effective amount of a formulation including a fibril or a fibril-forming peptide of SEQ ID NO: 1053 to the respiratory tract of the subject in need thereof, thereby targeting and activating B-1a lymphocytes in the pleural cavity of the subject and treating the medical condition.
2. A method of embodiment 1 wherein the medical condition including an inflammatory or autoimmune component is multiple sclerosis, rheumatoid arthritis, Huntington's disease, Parkinson's disease, amyotrophic lateral sclerosis, traumatic brain injury, cancer, ischemic reperfusion injury, arthritis asthma, or chronic obstructive pulmonary disease (COPD).
3. A method of targeting and activating B-1a lymphocytes and/or macrophages to suppress the immune system and treat a medical condition with an inflammatory or autoimmune component in a subject in need thereof including: administering a therapeutically effective amount of a formulation including a fibril or a fibril-forming peptide to the subject in need thereof, thereby suppressing the immune system and treating the medical condition in the subject.
4. A method of embodiment 3 wherein the medical condition is multiple sclerosis, rheumatoid arthritis, Huntington's disease, Parkinson's disease, amyotrophic lateral sclerosis, traumatic brain injury, cancer, ischemic reperfusion injury, arthritis asthma, or chronic obstructive pulmonary disease (COPD).
5. A method of embodiment 3 or 4 wherein the administering is to the respiratory tract.
6. A method of embodiment 1 or 5 wherein the administering to the respiratory tract is to the alveolar region of the lung.
7. A method of any of embodiments 3-6 wherein the fibril or fibril-forming peptide is SEQ ID NO: 1053.
8. A method of any of embodiments 3-6 wherein the fibril or fibril-forming peptide is a hexapeptide that oligomerizes with a Rosetta energy at or below −23 kcal/mol.
9. A method of any of embodiments 3-8 wherein the fibril or fibril-forming peptide includes one or more of SEQ ID NOs. 1-1062.
10. A method of any of embodiments 3-8 wherein the fibril or fibril-forming peptide includes one or more of SEQ ID NO: 5; SEQ ID NO: 1048; SEQ ID NO: 1049; SEQ ID NO: 123; SEQ ID NO: 1050; SEQ ID NO: 1051; SEQ ID NO: 791; SEQ ID NO: 1052; SEQ ID NO: 1053; SEQ ID NO: 1054; SEQ ID NO: 1055; SEQ ID NO: 1056; SEQ ID NO: 1057; SEQ ID NO: 220; and SEQ ID NO: 1058.
11. A method of any of the preceding embodiments wherein the formulation is a conductive formulation.
12. A method of any of the preceding embodiments wherein the formulation further includes a mucoactive or mucolytic agent.
13. A method of any of the preceding embodiments wherein the formulation is a dry powder formulation.
14. A method of any of the preceding embodiments wherein the formulation further includes a conductive agent.
15. A method of any of the preceding embodiments wherein said targeting and activating of B-1a lymphocytes and/or macrophages occurs in the pleural cavity.
16. A method of any of the preceding embodiments wherein the methods suppress the immune system.
17. A method of embodiment 16 wherein the suppressing the immune system includes suppression of one or more proinflammatory cytokines.
18. A method of embodiment 17 wherein the one or more proinflammatory cytokines are TNFα, IL-6, and/or IFNγ.
19. A conductive formulation including a fibril or fibril-forming peptide and a conductive agent.
20. A conductive formulation of embodiment 19 wherein the conductive agent is a hypertonic saline solution.
21. A conductive formulation of embodiment 19 or 20 further including a mucoactive or mucolytic agent.
22. A conductive formulation of embodiment 19, 20 or 21 wherein the fibril or fibril-forming peptide is a hexapeptide that oligomerizes with a Rosetta energy at or below −23 kcalmol.
23. A conductive formulation of embodiment 19, 20 or 21 wherein the fibril of fibril forming peptide is SEQ ID NO: 1053.
24. A conductive formulation of embodiment 19, 20 or 21 wherein the fibril or fibril-forming peptide includes a peptide of one or more of SEQ ID NOs: 1-1062.
25. A conductive formulation of embodiment 19, 20 or 21 wherein the fibril or fibril-forming peptide includes a peptide of one or more of SEQ ID NOs: SEQ ID NO: 5; SEQ ID NO: 1048; SEQ ID NO: 1049; SEQ ID NO: 123; SEQ ID NO: 1050; SEQ ID NO: 1051; SEQ ID NO: 791; SEQ ID NO: 1052; SEQ ID NO: 1053; SEQ ID NO: 1054; SEQ ID NO: 1055; SEQ ID NO: 1056; SEQ ID NO: 1057; SEQ ID NO: 220; and SEQ ID NO: 1058.

### Example 1

Amyloid fibrils, composed of hexapeptides, when injected into the peritoneum of mice, stimulate an immune suppressive response of sufficient magnitude to reduce the paralytic signs of experimental autoimmune encephalomyelitis (EAE). Analysis of the differential gene expression pattern in PBMCs revealed the amyloidogenic peptides, e.g. Tau 623-628 (SEQ ID NO: 123), induced a type 1 interferon (IFN) response. Plasmacytoid dendritic cells were the source of type 1 IFN, which was induced by NETosis arising from neutrophil endocytosis of the amyloid fibrils. Production of the type 1 IFN was therapeutic in Th1 induced EAE, but exacerbated the paralytic signs of Th17 induced disease. However, not all amyloidogenic peptides induced equivalent amounts of type 1 IFN. The induction of type 1 IFN appeared to correlate with the amount of fibril formation as measured by thioflavin T. A set of peptides with a polar fibril interface, e.g. Amylin 28-33 (SEQ ID NO: 1053), did not form measurable amounts of fibrils in physiological buffers, induced minimal amounts of type 1 IFN, but nevertheless were therapeutic, reducing IFNγ, TNFα, and IL-6 production by PBMCs and thus providing evidence of a second immune suppressive pathway. Further proof of the importance of this second immune suppressive pathway was the ability of amyloidogenic peptides to be therapeutic in IFNα/βR −/− animals with EAE.

To better define this second immune suppressive pathway, the induced effects of the amyloid fibrils at the site of injection in the peritoneum were investigated. The peritoneal cavity contains a variety of specialized cells including two types of resident macrophages (MΦs), large peritoneal MΦs (LPM) (CD11^{hi}F4/80^{hi}MHC-II⁻) and small peritoneal MΦs (SPM) (CD11b⁺F4/80^{lo}MHC-II^{hi}), B-1a lymphocytes (CD19^{hi}CD5⁺CD23), and more common components of blood, including the B-2 lymphocytes (CD19⁺CD5⁻CD23⁺), T lymphocytes, mast cells, neutrophils, eosinophils, and NK cells. The LPMs (F4/80^{hi}) are more prevalent than the SPMs (MHC-II), representing 90% of peritoneal MΦs. The B-1a and MΦs (LPM+SPM) each include 30% of the total peritoneal cells. Several groups have established that B-1a lymphocytes are distinguishable from the more plentiful B-2 lymphocytes and are enriched in body cavities. The chemokines and integrins, which are responsible for B-1a localization to the peritoneal cavity and their exodus when activated, have also been defined. The B-1a cell population is notable for its constitutive expression of IL-10, a well-established immune suppressive cytokine. IL-10 producing B cells, B10 cells, were initially shown by Janeway and colleagues to be necessary for the recovery from the signs of EAE and were subsequently demonstrated to be immune suppressive in animal models of multiple sclerosis, inflammatory bowel disease, collagen induced arthritis, lupus, stroke, insulin resistance, and allergic airway disease. The B10 cells in many of these studies were isolated from the spleen and not the peritoneal cavity.

Maximal immune suppression by B10 cells is observed after the cells are activated through TLR, CD40, or IL-21 ligation, all of which induce both an increase in IL-10 production and an egress of the cells from the peritoneum into secondary lymph organs. Reduction of symptoms in each of the inflammatory, autoimmune diseases correlated with reduction of TNFα, IL-6, and IFNγ, a pattern similar to that seen with the administration of the amyloidogenic peptides.

Methods.

Induction of active EAE in mice by immunization with MOG and adjuvant. EAE was induced in female wild type C57Bl/6 mice or μMT and IL-10 deficient mice on C57B/6 background (Jackson Laboratories) by procedures previously described. Briefly, EAE was induced at 9 weeks of age by subcutaneous immunization in the flank with an emulsion containing 200 μg myelin oligodendrocyte glycoprotein₃₅₋₅₅ (MOG₃₅₋₅₅; MEVGWYRSPFSRWHLYRNGK (SEQ ID NO: 1065)) in saline and an equal volume of complete Freund's adjuvant containing 4 μg/ml mycobacterium tuberculosis H37RA (Disco Laboratories). All mice were given 400 ng of pertussis toxin (List Biological) intraperitoneal (i.p.) at 0 and 48 h post-immunization. The signs of neurological impairment were scored as follows: 0, no clinical disease; 1, tail weakness; 2, hindlimb weakness; 3, complete hindlimb paralysis; 4, hindlimb paralysis and some forelimb weakness; 5, moribund or dead. When animals exhibited an average of level one to two for clinical signs they were injected in the peritoneum with 10 μg of Tau 623-628, Amylin 28-33 peptide (SEQ ID NO: 1053), or PBS daily. For intranasal inoculation, 10 μg of Amylin 28-33 in 10 μl PBS was gradually released into the nostrils of anesthetized mice. All animal protocols were approved by institutional IACUC.

Adoptive Transfer of B-1a Lymphocytes.

B-1a lymphocytes were purified by cell sorting of peritoneal cells from wild type C57Bl/6 mice. Ten days following induction of active EAE in μMT mice, 3.5×10⁵ B-1a lymphocytes were transferred into the peritoneal cavity. Mice were treated with 10 μg Amylin 28-33 or PBS daily for 14 days. Control μMT mice with EAE were treated with 10 μg Amylin 28-33 without transfer of B-1a lymphocytes. Mice were examined daily for clinical signs of EAE and were scored on a five point scale described above.

Microscopy.

C57B/6 female mice were injected with FITC-Tau 623-628, and peritoneal cells were isolated after 10 minutes, washed, stained with rat antimouse CD19 (PE), F4/80 (Alexa Fluor 647), and DAPI, washed, and plated on polylysine coated microscope slides. Cells were visualized using a Leica TCS SP8 white light laser confocal microscope.

Bioluminescence Experiments.

B-1a lymphocytes and peritoneal MΦs were purified by cell sorting of peritoneal cells isolated from luciferase transgenic mice ((B6.FVB-Ptprca Tg(CAG-luc,-GFP)L2G85Chco Thy1a/J), which express the CAT-luc-eGFP, L2G85 transgene). In the initial experiment 2×10⁵ and 1×10⁵ B-1a lymphocytes were injected in the peritoneum of C57BL/6 female albino mice (B6(Cg)-Tyr^{c-2J}/J), followed by injection of 10 μg of Tau 623-628 to activate the lymphocytes, and 0.3 mg/g body weight of luciferin substrate (D luciferin firefly L-8220 Biosynth) to initiate the bioluminescence imaging. The location of the luciferase expressing cells was measured by imaging every 5 minutes for 75 minutes.

To confirm the diminution of luminescence was due to the trafficking of the luc⁺ cells, and not the degradation of the luciferin, two C57BL/6 albino mice were injected with 10 luc⁺ peritoneal MΦs, a third with 2×10⁵ B-1a lymphocytes, and fourth mouse serving as a BLI control was injected with luciferin only. The mice were subsequently injected with 10 μg of Tau 623-628 and 0.3 mg/gram of luciferin. Bioluminescence was measured immediately after the injection of luciferin and 5 minutes later. After thirty minutes the mice were reinjected with luciferin and the resultant luminescence measured. A similar injection and measurement was done after 60 minutes. The multiple injections of luciferin kept the drug level close to saturation during all measurements, so that any changes were due to cell movement. Mice were imaged using an IVIS100 charge-coupled device (CCD) imaging system (Xenogen, Alameda, Calif.). Imaging data were analyzed and quantified with Living Image software 4.4 (Xenogen).

Peptide Synthesis and Preparation of FITC-Tau.

Peptides were synthesized using solid phase techniques and commercially available Fmoc amino acids, resins, and reagents (PE Biosystems, Foster City Calif., and Bache, Torrance, Calif.) on an Applied Biosystems 433A peptide synthesizer as previously described. Wender et al. (2000) Proc. Natl. Acad. Sci. USA 97(24):13003-13008. Purity of the peptides was shown to be greater than 90% using a PE Biosystems 700E HPLC and a reversephase column (Alltech Altima). The molecular weight of the peptides was confirmed using matrix assisted laser desorption mass spectrometry. To prevent excess amounts of fluorophore in the fibrils, Tau 623-628 was mixed at a ratio of 10:1 with an analog with FITC attached to the amino terminus of Tau 623-628 with an amino caproic acid linker. The resulting fibril mixture is referred to in the text as FITC-Tau.

RNA Isolation, Chip Hybridization, and qPCR.

Total RNA was extracted from FACS purified B-1a lymphocytes and peritoneal MΦs pooled from 3-6 C57BL/6 female mice injected with 10 μg of LPS, Tau 623-628, Amylin 28-33, or PBS using Trizol reagent and the Qiagen RNeasy micro kit. First strand cDNA was synthesized with 30-50 ng of total RNA using Superscript III first strand synthesis supermix for qRT-PCR. QPCR assays were performed using the 7900HT Fast Real Time PCR System (Applied Biosystems), and the Taqman Gene Expression Arrays (Applied Biosystems) using commercially available primers (ABI). All assays were performed according to manufacturer's instructions. The comparative Ct method for relative quantification (ΔΔCt) was used to compare gene expression. Housekeeping gene expression was used to normalize expression using the following equation: Normalized expression=2^{[Ct(house-keeping gene)-Ct(gene)]}.

Gene expression changes associated with treatment with LPS, Amylin 28-33, and Tau 623-628, were quantified using a microarray (SurePrint G3 Mouse; Agilent Technologies). RNA quality was shown to be suitable for microarray experiments (2100 Bioanalyzer, Agilent Technologies, Inc.). Analysis and quantitation of the data were done using GeneSpring and Ingenuity software.

Flow Cytometry.

Peritoneal cavity cells were obtained by flushing the peritoneal cavity with 10 ml of cold PBS containing 0.1% bovine serum albumin and 5 mM EDTA. Single-cell suspensions were stained with the following fluorochrome conjugates: CD5 (PE Cy5, PE, or APC), CD19 (PE Cy5.5, Pacific Blue, or APC), CD11b (Pacific Blue or PE), F4/80 (APC), CD21 (APC), CD23 (PE), Gr-1 (PECy7), B220 (APC-Cy7), CD80/86 (biotin-Qdot605-streptavidin), CD4 (FITC), CD3 (PerCP-Cy5.5), and IgM (Alexa Fluor 700). Sorting of cells used a FACS-Aria or a Fortessa (BD) equipped with four lasers and optics for 22-paramenter analysis. Analysis was done using FlowJo.

TLR Binding Assays.

Commercially available HEK293 cells transfected with murine TLR4, MD-2, CD14, or TLR2 and an inducible secreted embryonic alkaline phosphatase (InVivoGen) were plated in a 96 well plate. The SEAP reporter gene in the cells is under the control of an IL-12 p40 minimal promoter fused to five NF-κB and AP-1-binding sites. Stimulation with either a TLR4 or TLR 2 ligand activates NF-κB and AP-1, which induces the production of SEAP. Levels of the secreted alkaline phosphatase measured in the cell culture medium, is proportional to the stimulation of the TLR pathway. Background levels are measured using HEK-Blue Null cells, which are transfected with the alkaline phosphatase, but not the TLR receptor. The TLR4 transfected cells were grown to confluence and 2, 1, 0.2 μg of LPS, or 10 μl of a set of amyloidogenic peptides were added to each well in duplicate in HEK-Blue detection medium (InVivoGen). In the case of the TLR2 transfected cells, PAM2CSK4 was the positive control, and only 2 μg of LPS were assayed. The plates were incubated for 12 hours at 37° C., and the resulting blue color measured by reading the absorption at 650 nm.

Results.

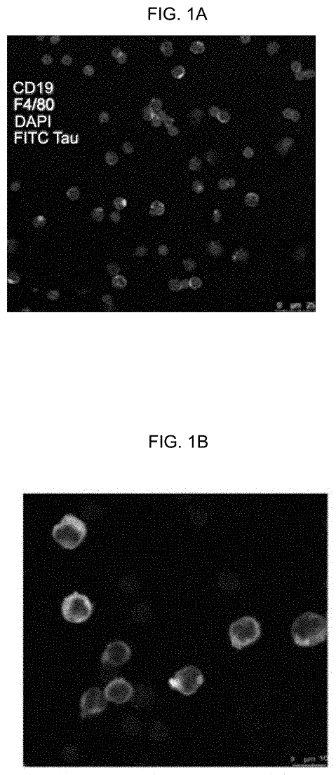

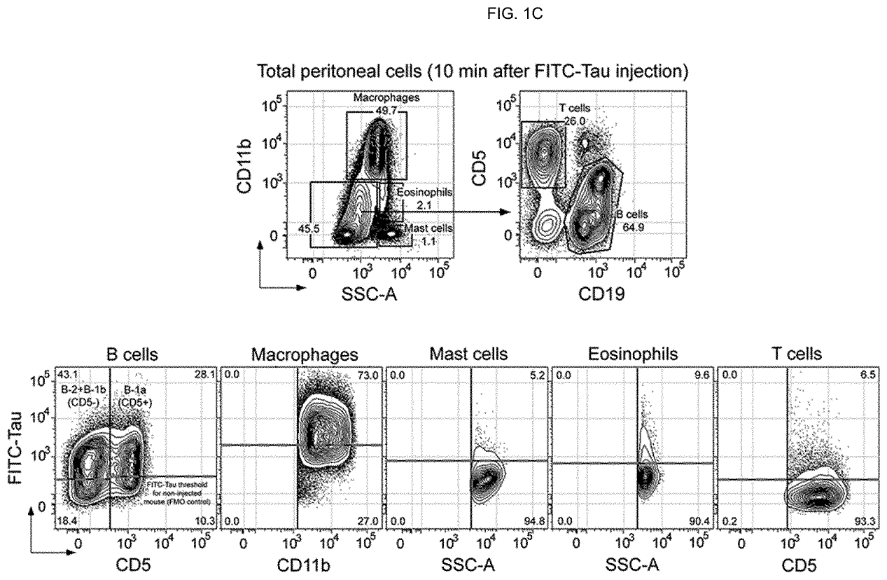

Amyloid fibrils are endocytosed by peritoneal B cells and MΦs. Amyloid fibrils are endocytosed by MΦs, dendritic cells, microglia, and neutrophils. To determine whether peritoneal cells bind the amyloid fibrils, fluorescently labeled Tau 623-628 was mixed with unlabeled peptide at a 1:10 ratio, and the resultant fibrils injected in the peritoneum of healthy, wild type C57BL/6 mice. After twenty minutes the peritoneal cells were collected by lavage, stained with anti-CD19 and anti-F4/80, and layered on poly-lysine coated slides. Confocal microscopy was performed and revealed the presence of intracellular fluorescent fibrils, suggesting that the fibrils were bound and were endocytosed by both B cells (CD19⁺) and MΦs (F4/8⁺) (FIGS. 1A and B). Viewing multiple fields revealed that the majority of the fluorescent fibrils were bound by F4/80⁺ cells with a smaller percentage binding CD19⁺ B cells.

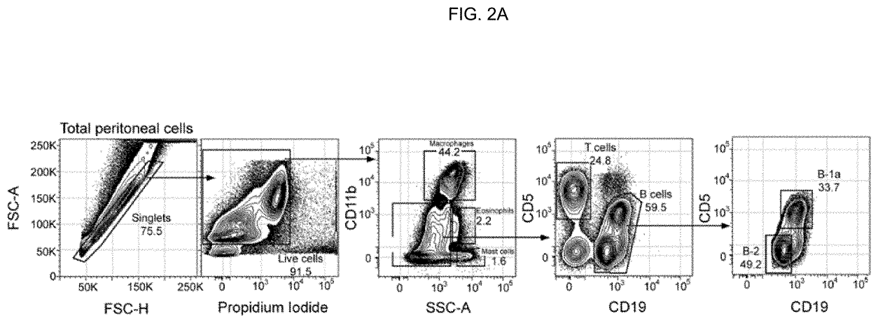

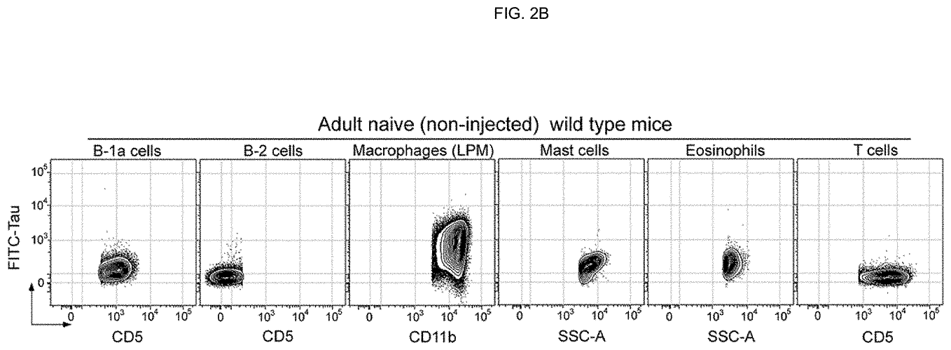

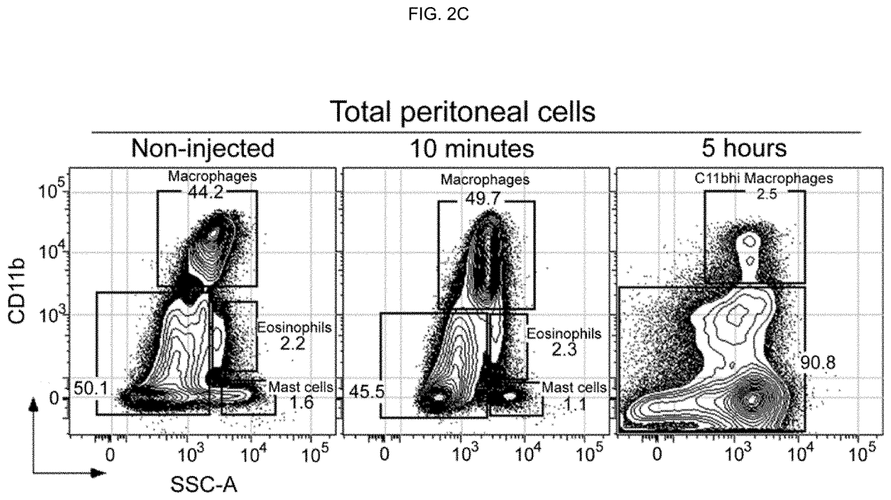

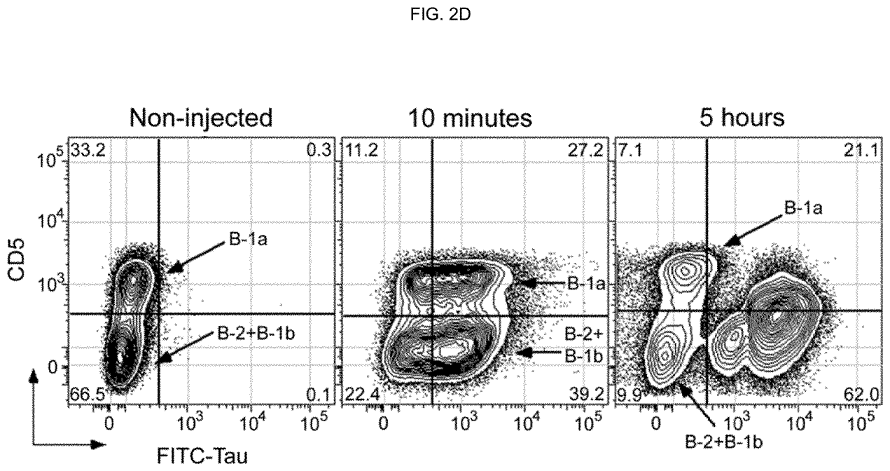

Analysis of peritoneal cells isolated from mice injected with the fluorescent amyloid fibrils by flow cytometry confirmed and extended the microscopic study. The composition of the various cell types in the peritoneum can be delineated using 10-color, 12-parameter hi-dimensional analysis (FIGS. 2A and 2B). When the peritoneal MΦs (both SPM and LPM), mast cells, T, B-2, and B-1 lymphocytes are delineated with antibodies against CD11b, CD5, and CD19, a more complex pattern of uptake and trafficking is apparent (FIG. 1C). Within 10 minutes of the FITC-Tau injection more than 70% of the B-1 and B-2 lymphocytes and LPM are FITC positive. T lymphocytes and mast cells are minimally stained, demonstrating specific binding or uptake by B cells and MΦs. Five hours after injection of the amyloid fibrils an interesting pattern emerges. The majority of the CD11b high population is significantly reduced from 45% to 3% of total peritoneal cells (FIG. 2C). Most of the B-1a population has disappeared, with the remaining cells being FITC-Tau negative (FIG. 2D). The majority of T lymphocytes and mast cells remained unstained with the fibrils. Collectively, the flow cytometry studies revealed that B cells and MΦs bind the fibrils, and that the relative number of B-1a lymphocytes (CD19^{hi}CD5⁺) and the LPMs (CD11b^{hi}) was dramatically reduced 5 hours after injection of the amyloid.

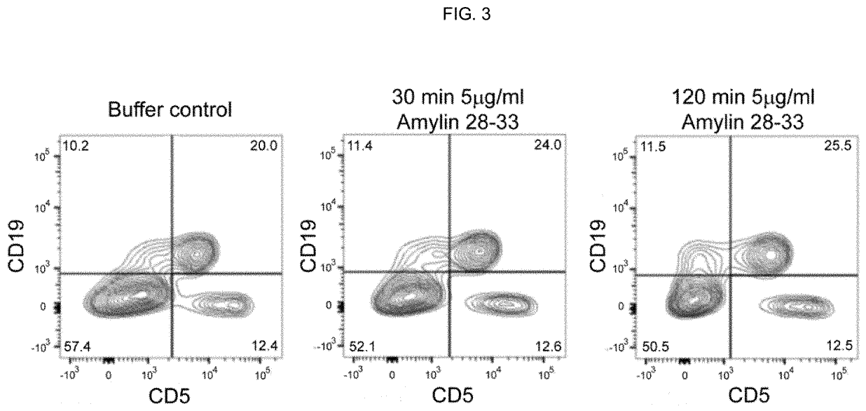

Whether fibrils were selectively toxic to the B-1a and LPMs was evaluated. This was ruled unlikely when no cell death was observed when the peritoneal cells were cultured in vitro with the fluorescent amyloid (FIG. 3). Rather than killing the cells, a testable hypothesis was that the fibrils induced the exodus of the two cell populations from the peritoneum.

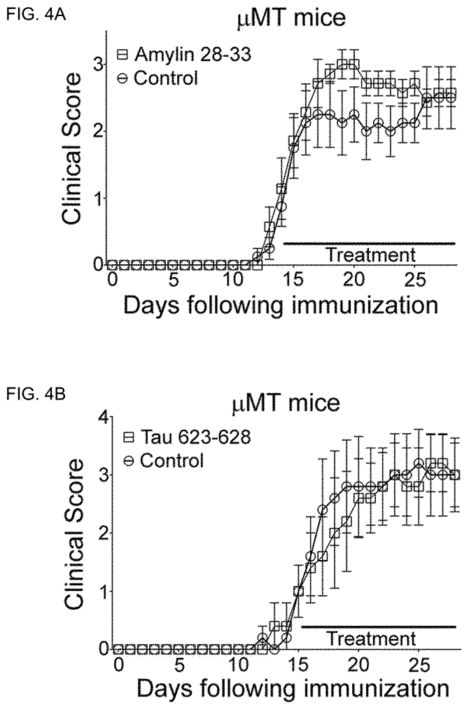

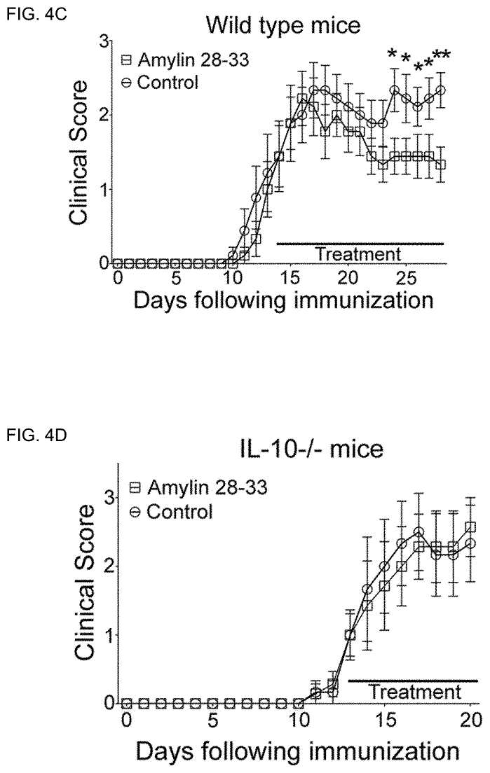

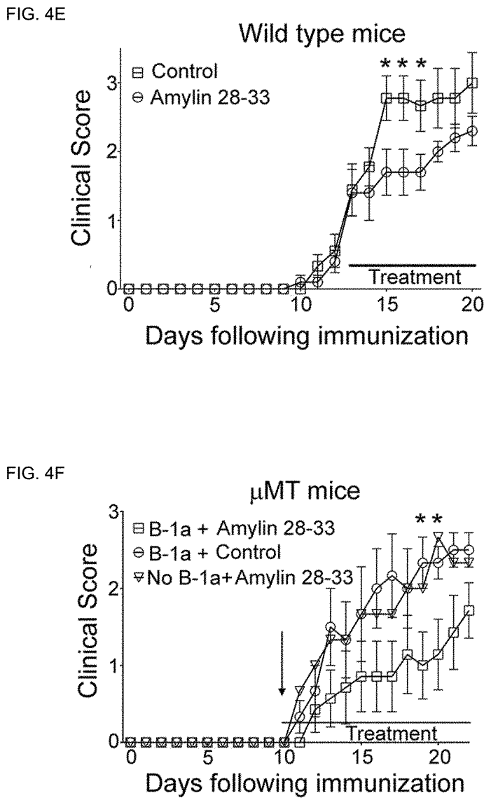

μMT and IL-10 knockout mice do not respond to therapy. To determine the importance of B lymphocytes in the mode of action of the amyloid fibrils, EAE was induced in μMT mice, which due to a mutation in the transmembrane region of IgM lack expression of all subtypes of B cells. The paralytic signs of the disease were induced in these animals, consistent with induction of the disease by T lymphocytes, but neither Tau 623-628, nor Amylin 28-33 was therapeutic when compared to the effect seen in wild type mice (FIGS. 4A-4C). The therapeutic activity of the amyloidogenic peptides appeared to require the presence of B cells, most likely B-1a lymphocytes based on the composition of the peritoneum and the microscopy.

B-1a lymphocytes are characterized by the constitutive expression of relatively large amounts of IL-10. To establish whether this cytokine was central to therapeutic effects of the peptides and to correlate the activity with this B cell subtype, 10 μg Amylin 28-33 was used to treat EAE induced in IL-10 knockout animals (FIGS. 4D and 4E). Again, the peptide was ineffective in this animal model, establishing that both IL-10 and B lymphocytes are central to the therapeutic activity of the amyloidogenic peptides.

Adoptive transfer of B-1a lymphocytes restored therapeutic efficacy of amyloidogenic peptides in μMT animals. The characteristics of peritoneal B-1a lymphocytes clearly correlate with the apparent requirements for therapeutic function. Thus replacement of this population in μMT mice should restore the immunosuppressive activity of the amyloid fibrils. Peritoneal cells were isolated from C57BL/6 mice, and B-1a lymphocytes (CD19^{hi}CD5⁺) were purified by cell sorting. EAE was induced in μMT mice and, on day 10 after induction, prior to the appearance of clinical signs, the mice were injected in the peritoneal cavity with purified B-1a lymphocytes (3.5×10⁵ cells). The mice were divided into two groups and treated daily with Amylin 28-33 (10 μg) or buffer alone (FIG. 4F). μMT mice induced with EAE that did not receive B-1a lymphocytes but were treated with Amylin 28-33 were used for comparison. Interestingly, only mice that received the transfer of B-1a lymphocytes treated daily with the amyloidogenic peptide exhibited reduced paralytic signs of EAE. Adoptive transfer of untreated B-1a lymphocytes was as ineffective as buffer control, establishing not only the importance of B-1a lymphocytes, but that the cell population needs to be activated by the fibrils to be effective. This corroborates studies that regulatory B cells are more potent suppressors of autoimmunity than their non-activated counterparts.

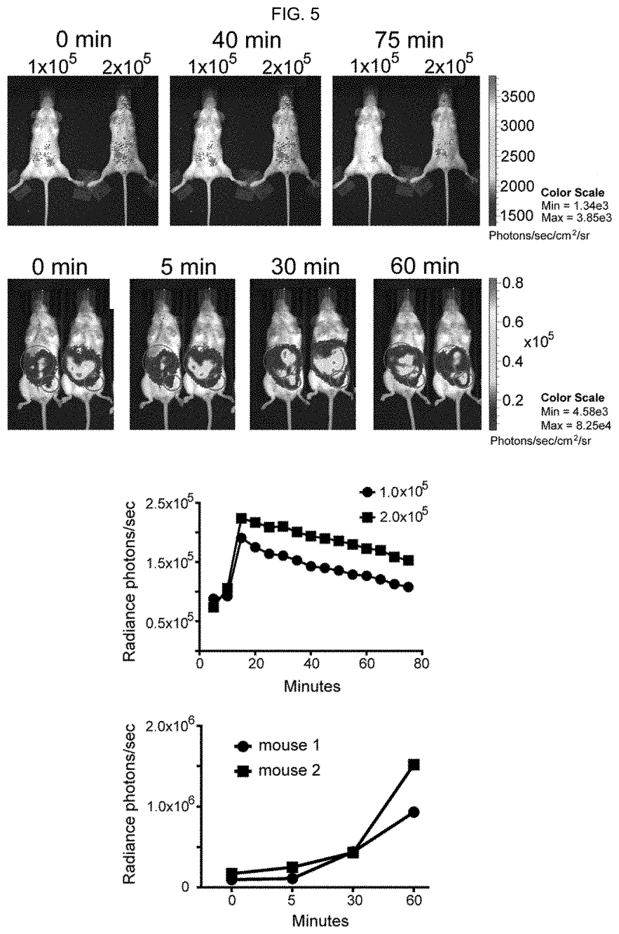

Amyloidogenic peptides induce exodus of B-1a lymphocytes and LPM from the peritoneal cavity. Real time measurement of bioluminescence demonstrated that the amyloidogenic peptides induce an exodus of both B-1a lymphocytes and LPM from the peritoneum. B-1a lymphocytes (CD19^{hi}CD5⁺CD23⁻) and LPMs (CD11bhiF4/80hi) were sorted from peritoneal cells isolated from luciferase transgenic mice, C57BL/6 L2G85 (H-2d) (B6.FVB-Ptprca Tg(CAG-luc,-GFP)L2G85Chco Thy1a/J), that express the CAT-luc-eGFP, L2G85 transgene. In the initial experiment 1×10⁵ and 2×10⁵ B-1a lymphocytes were injected into the peritoneum of C57BL/6 female albino mice, followed by injection of 10 μg of Tau 623-628 to activate the lymphocytes, and 300 μg/g body weight of the substrate luciferin, to initiate the enzyme-substrate reaction for the bioluminescence imaging. The location of the luciferase expressing cells was monitored by imaging the mice every 5 minutes for 75 minutes (FIG. 5, top left). The diffuse distribution of the luminescence, corresponding to the peritoneal cavity, seen at early times, was reduced in intensity over time, with focal regions of intensity appearing to localize in inguinal lymph nodes beginning at 35 minutes (FIG. 5, top left). Measuring the total number of photons per second in the abdominal region revealed a rapid, dose dependent, reduction of light emitted over the 75 minutes of the experiment (FIG. 5, top right). Even though the size of the signal was proportional to the number of cells injected, the rate of reduction, or slope of the curve, was equivalent in the two animals. Such a result is consistent with the amyloid fibrils triggering egress of the B-1a lymphocytes from the peritoneum.

The experimental design of the initial experiment did not allow the reduction of the signal to be assigned unequivocally to the migration of the lymphocytes because the concentration of the luciferin also diminishes during the 75 minutes of examination. To better assign the basis of the reduction of luminescence to the migration of the luc⁺ cells, a second experiment was performed with four recipient mice. Two were injected with 1×10⁶ luc⁺ MΦs (LPMs), a third with 2×10⁵ B-1a lymphocytes, and fourth mouse serving as a control was injected only with luciferin. In this experiment, the mice were subsequently injected with 10 μg of Tau 623-628 and luciferin. Bioluminescence was measured immediately after the injection of luciferin and 5 minutes later. After thirty minutes the mice were reinjected with luciferin and the resultant luminescence measured. A similar injection and measurement was done after 60 minutes. The multiple injections of luciferin kept the drug level close to saturation during all measurements, so that any changes were due to migration and trafficking. Using 1×10⁸ LPMs produced a vivid signal at all time points, whose details were similar to that observed in the initial experiment; increased distribution of light with time, along with a concentration in an area over inguinal lymph nodes (FIG. 5, bottom left). In the mouse receiving the B-1a lymphocytes, a less intense signal was observed, but nevertheless evidence of exodus of the luc⁺ cells from the peritoneum was apparent. When photons per second over the full body of each mouse were measured and plotted versus time, there was a proportional increase in signal as a function of time. In the case of the area corresponding to the inguinal lymph nodes, there was close to a 10-fold increase in luminescence from the measurement at 5 to 60 minutes (FIG. 5, bottom right). Collectively the imaging experiments establish that the amyloidogenic peptides induce a migration of both B-1a lymphocytes and LPMs from the peritoneum to the inguinal lymph nodes.

To further examine how amyloid fibrils induce an exodus of B-1a lymphocytes and LPMs a series of experiments was performed using IL-10 reporter mice in which IL-10 and GFP are connected via an internal ribosome entry site (IRES) to create a bicistronic message marking IL-10 secreting cells with fluorescence (Bouabe (2012) Scand. J. Immunol. 75(6):553-567). Because LPS induces B-1a cell migration from the peritoneum to the spleen, the effects of the amyloid fibrils were compared to those effects observed with the TLR4 ligand (Balabanian et al. (2003) J. Immunol. 170(6):3392-3400; Ghosn et al. (2011) Proc. Natl. Acad. Sci. USA 108(7):2879-2884). To allow for maximal changes in IL-10 transcription, and because the resulting GFP is relatively long-lived, the experiment was designed to confirm exodus, and to identify possible sites of migration. Because previous experiments established that 80% of the B-1a and LPMs exited the peritoneum at 5 hours, time points for analysis were chosen at 30 minutes and at 24 hours after injection of the fibrils. Three groups of three IL-10 reporter C57BL/6 female mice were injected with 10 μg LPS, 10 μg Amylin 28-33, or buffer alone and after 5 or 24 hours the peritoneal cells were lavaged, the spleen and inguinal and axillary lymph nodes were dissected with the lymph nodes being pooled, and single cell suspensions were prepared and delineated using 10-color, 12-parameter hi-dimensional analysis. Similar cells and tissues also were taken from three wild type mice as additional controls. Injection of LPS not only increased the relative numbers of IL-10-secreting B-1a lymphocytes (from 25% to 40% in 24 h), but it also induced higher levels of IL-10 as evidenced by higher levels of GFP median fluorescence intensity (MFI). In contrast, injection of Amylin 28-33 did not increase IL-10 secretion, however there was a decrease in the number of IL-10⁺ B-1a lymphocytes and LPMs in the peritoneum after 24 hours that was not observed with LPS.

In the spleen, LPS induced an increase in both the number of B-1a lymphocytes and the amount of IL-10 expressed per cell 24 hours after injection. Amylin fibrils did not induce an increase of the B-1a lymphocytes in spleen, but rather an apparent reduction in both numbers and IL-10 expression. The opposite pattern was observed in the pooled lymph nodes. LPS resulted in a slight increase of B-1a lymphocytes in the pooled lymph nodes. Injection of Amylin 28-33 increased the percentage of B-1a lymphocytes in the lymph node, with an increase in the amount of IL-10 expression. In IL-10 reporter mice with EAE, an additional pattern was observed. No significant increase in B-1a lymphocytes were detected in the brain or spinal cord, consistent with the hypothesis that the B-1a and Mm populations migrate to the secondary lymph organs, and not to the primary sites of inflammation.

The experiments using the IL-10 reporter mice revealed that both LPS and the fibrils activated B-1a lymphocytes, SPM, and LPM, inducing IL-10 gene expression, and subsequent exodus from the peritoneum. However the magnitude of the increase and the details of the directions of migration differ. As previously reported, LPS activates the B-1a lymphocytes resulting in greater production of IL-10 and migration to the spleen, whereas the fibrils predominantly induced migration to the lymph nodes. Interestingly only B-1a lymphocytes expressing CD80/86 secrete IL-10, and IL-10 secreting B-1a lymphocytes were found in lymph nodes of normal animals with the relative number increased with injection of amylin fibrils. The flow cytometry experiments establish that the amyloidogenic peptides activate both B-1a lymphocytes and LPMs, resulting in both cell types trafficking to the draining lymph nodes.

Differential gene expression in B-1a lymphocytes and MΦs induced by amyloid fibrils. The migration of B-1a lymphocytes and LPMs after the injection of amyloid fibrils was consistent with the activation of both cell types. However, the receptor(s) for the fibrils has not been defined for either the B-1a lymphocytes or the peritoneal MΦs. Unlike migration induced by LPS stimulation, the fibrils composed of the peptides do not bind to TLR2 MD2/TLR4. A set of over fifteen different amyloidogenic peptides was screened for binding to commercially available HEK cells transfected with murine TLR2 or MD2/CD14/TLR4. The transfected cells contained a secretable form of alkaline phosphatase under the control of an NFkB promoter. None of the amyloid fibrils composed of the varying peptides was positive in this assay.

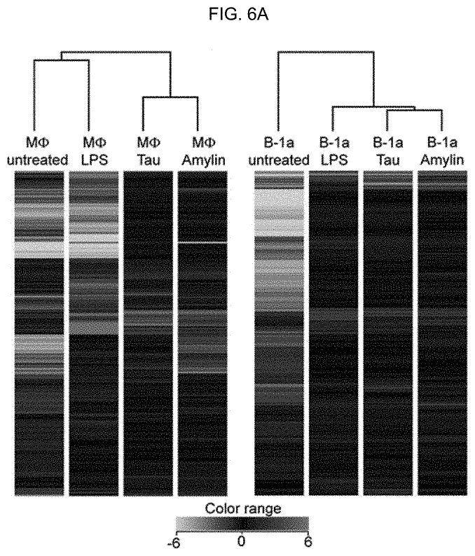

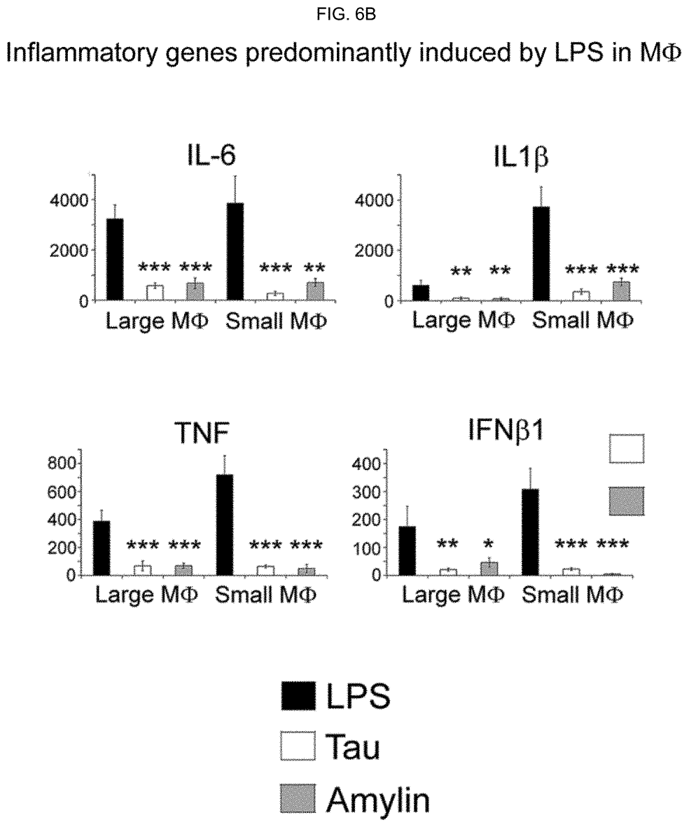

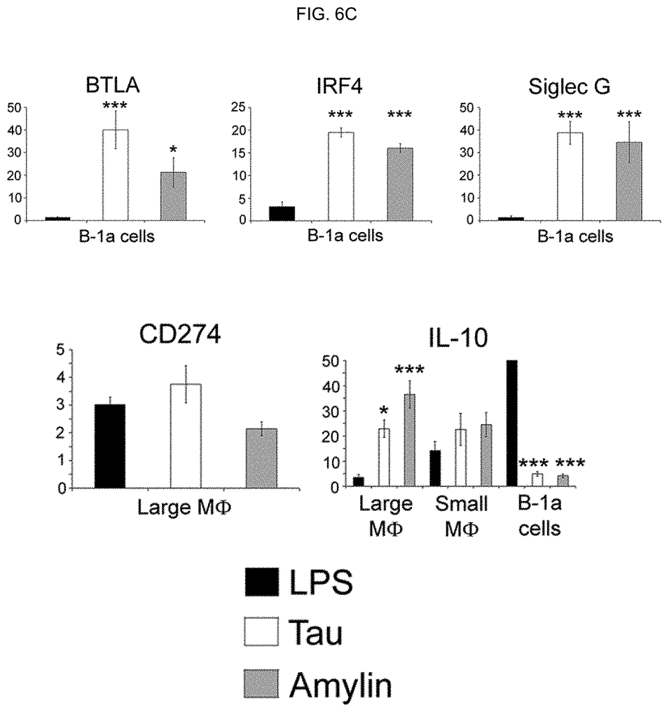

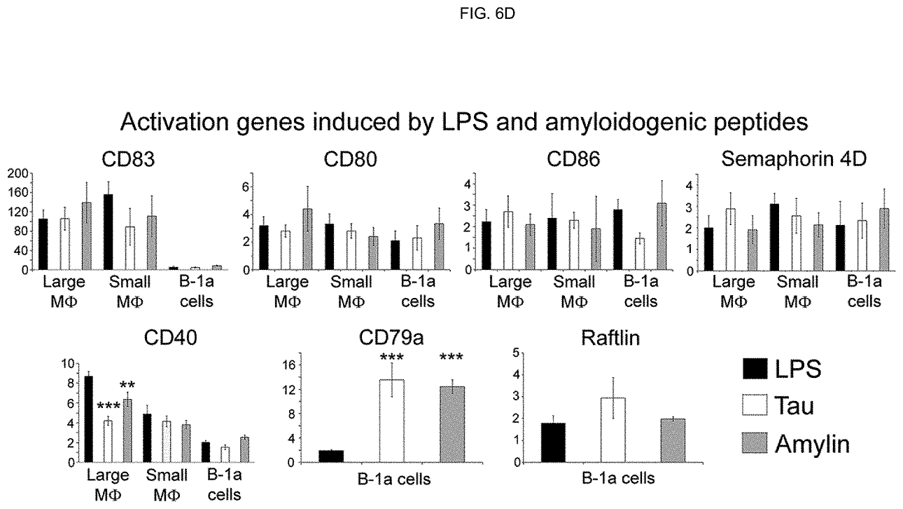

To confirm and increase the understanding of how the fibrils are activating the peritoneal cells, differential gene induction in purified B-1a and LPMs was analyzed. Making such measurements was complicated by the fact that LPS and the fibrils induce a rapid migration of the relevant cells from the peritoneal cavity, and consequently a high percentage would not be isolated by lavage an hour after injection. To minimize the population bias, and yet allow sufficient time for the fibrils to induce gene expression, cells were isolated between 30-40 minutes after injection of LPS or the amyloidogenic peptides. Consequently, the analysis is limited to gene expression in the 30-40 minutes after stimulation. Peritoneal cells from groups of three C57BL/6 female mice were isolated after injection with either LPS, fibrils composed of Amylin 28-33 or Tau 623-628, or buffer control. B-1a lymphocytes (CD19^{hi}CD5⁺CD23⁻) and LPMs (CD11b^{hi} MΦs) from the four groups of three mice were sorted into Trizol, RNA extracted, and gene expression measured using a murine Agilent whole genome expression microchip. Differential gene expression of the B-1a and LPMs was calculated by subtracting the gene expression data from cells isolated from mice injected with buffer from expression data from mice injected with LPS or the amyloid fibrils (FIG. 6A). All microarray data are available at the Gene Expression Omnibus (GEO) database (GEO series accession number: GSE73026).

The pattern of gene expression induced by LPS is well characterized in both cell types binding to CD14/TLR4 (Kawai & Akira (2010) Nat. Immunol. 11(5):373-384; Rossol, et al. (2011) Critical reviews in Immunol. 31(5):379-446) resulting in the induction of a wide spectrum of proinflammatory mediators such as IL-6, TNFα, type 1 IFN, Serpins, IL-1α and β, chemokines CXCL10, CXCL3, MYD88, and over 50 genes known to be induced by RelA/p65 NFkB (Bode et al. (2012) Cellular Signalling 24(6):1185-1194). In peritoneal MΦs amyloidogenic peptides stimulated a distinctly different set of genes from those induced by LPS. To portray the variation, a heatmap of the correlation of a set of 730 annotated genes induced by LPS in MΦs demonstrates the gene expression signature induced by the two amyloidogenic peptides is similar and distinct from the pattern generated by injection with LPS (FIG. 6A). The majority of genes preferentially induced by the fibrils corresponded to MΦ stimulation, cytokine production, oxidative phosphorylation, and mitochondrial dysfunction pathways. The oxidative phosphorylation pathways were induced, demonstrated by the expression of a large number of mitochondrial genes composing the five complexes involved in mitochondrial electron transport and ATP production, characteristic of amyloid fibril interactions with mitochondria (DuBoff B, Feany M, & Gotz J (2013) Trends in Neurosciences 36(6):325-335).

In the case of B-1a lymphocytes, a less vivid difference between LPS and the peptides is seen, but the patterns induced by the two types of fibrils are distinguishable from that of LPS. In contrast, with MΦs, LPS and the amyloidogenic peptides both induced a pattern of expression characteristic of B cell activation. The amyloidogenic peptides stimulated CDC42 small effector protein) Calcium release (stim 1, orai 1 and 3), BcR (CD79, Syk, Lyn, PI3K, Akt, m-Tor, Bcl2A1d, c-src, PTEN, and Vav-1) and CD40 signaling (Traf 2, 4, and 5), all of which induce NFκB activation. Even though the LPS and amyloidogenic peptides induced a large number of similar genes involved in lymphocyte activation, a clear distinction could be observed, with the amyloidogenic peptides inducing a set of immunosuppressive proteins such as BTLA, IRF4, and Siglec G.

To confirm the gene expression data from the chip, a set of genes was analyzed by qPCR (FIGS. 6B-6D). In these experiments RNA was isolated from B-1a, large and small MΦs using identical methods and similar times after injection of the stimulants as was done for the microarray. Consistent with the pathway analysis of the chip data, IL-6, TNF, IL-1β, and IFNβ1 were significantly induced by LPS in the peritoneal MΦs, and minimally by the peptide fibrils. Interestingly, the SPMs (CD11b⁺F4/8^{lo/-} MΦs) uniformly expressed greater amount of the inflammatory genes, particularly L-1β, than the LPMs (CD11^{hi}F4/80^{hi} MΦs). In the peritoneum, SPMs compose less than 10% of the MΦ population, are not the dominant cell type that endocytose the fibrils, nor do they represent the major depleted population of cells after injection of LPS or the fibrils. In contrast, the fibrils induced a set of genes associated with immune regulation, BTLA, Siglec G, and IRF4 in B-1a lymphocytes, and CD274 in LPMs. LPS induced some, but not all of these genes. The third set of genes analyzed were those known to be associated with cell activation. CD40, CD80, CD86, and semaphorin 4D were induced by both LPS and the fibrils in B-1a lymphocytes and both types of MΦs. CD83 was induced by both stimuli principally on the MΦs, while CD79a and Raftlin were induced on the B-1a lymphocytes.

The pattern of gene expression indicated that both types of amyloid fibrils activated the B-1a lymphocytes and both populations of the peritoneal MΦs (SPM and LPM). IL-10 gene expression was increased in both B-1a and LPMs, two of the cell types shown to traffic to lymph nodes. The induction of BTLA and Siglec G in the B-1a lymphocytes would increase their immune regulatory phenotype. The expression of IL-10 in the LPMs is consistent with the conversion of these cells to a M2 phenotype, also believed to suppress inflammatory responses.

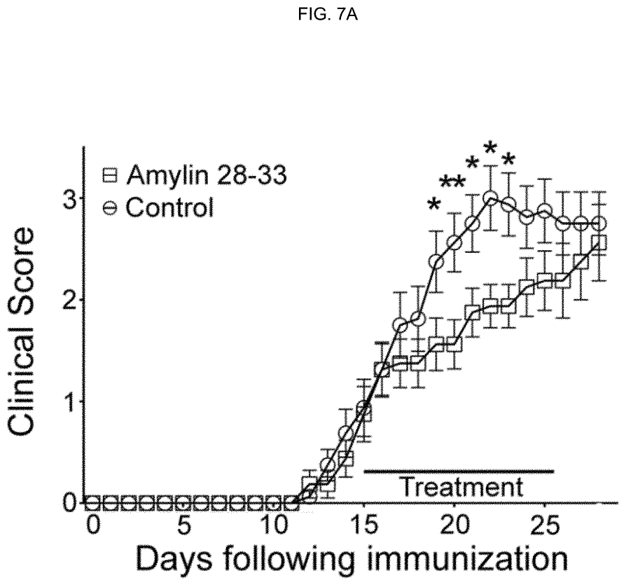

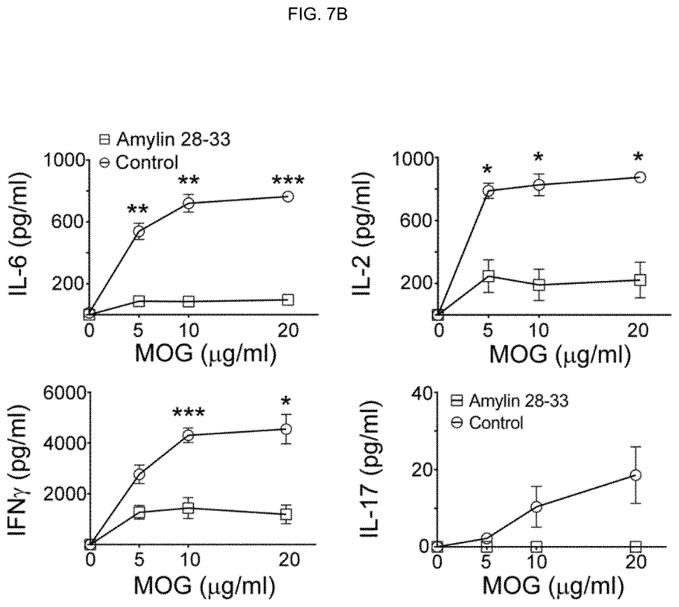

Delivery to the respiratory tract retains the therapeutic efficacy of the amyloidogenic peptides. Peritoneal injection is not a practical route of drug administration for activation of B-1a lymphocytes in humans. However, B-1a lymphocytes also are plentiful in the pleural cavity of both mice and humans (Yenson V & Baumgarth N (2014) Methods Mol. Biol. 1190:17-34). To examine whether this alternative route of administration is both practical and sufficient for treatment, 10 μg Amylin 28-33 was administered daily intranasally to groups of 10 C57BL/6 mice with EAE. The paralytic signs of the disease were reduced in a fashion equivalent to that seen when the amyloidogenic peptide is injected i.p. (FIG. 7A). In addition, splenocytes from peptide treated mice exhibited a reduction in secretion of proinflammatory cytokines, IL-6, IFNγ, IL-2, and IL-17, in response to MOG₃₅₋₅₅ challenge in vitro, compared to control (FIG. 7B), a pattern identical to when Amylin 28-33 was injected (Kumellas et al. (2013) Sci. Transl. Med. 5(179):179ra142).

The success of delivery to the respiratory tract is consistent with a mode of action in which the B-1a lymphocytes play a central role, but also establish a potential route of administration that can be used in clinical trials in human patients.

Discussion.

Amyloid fibrils composed of amyloidogenic peptides exhibit wide spectrum of biological activities, the sum of which results in an immune suppressive response of sufficient magnitude to be therapeutic in a robust model of multiple sclerosis. As molecular chaperones they bind a spectrum of proinflammatory mediators in plasma. In blood they are endocytosed by neutrophils, which induce the production of nets, which in turn induces plasmacytoid dendritic cells to secrete type 1 IFN. Here it is shown that fibrils bind and activate both B-1a lymphocytes and a subset of peritoneal MΦs known as large peritoneal MΦs (LPMs) (Ghosn E E, et al. (2010) Proc. Natl. Acad. Sci USA 107(6):2568-2573), which are induced to increase IL-10 transcription and migrate out of the peritoneum to secondary lymph organs. The exodus results in the selective delivery of IL-10 to immunological sites shared with inflammatory T lymphocytes and their complementary antigen presenting cells. IL-10 effectively inhibits both inflammatory cell populations, with reduction of the production of proinflammatory cytokines, IL-6, TNFα, and IFNγ. This reduction of cytokines was the hallmark of the immune suppression induced in EAE by the amyloidogenic peptides. The peritoneal cells do not appear to migrate to the sites of inflammation in the CNS, and consequently do not need to cross the blood-brain barrier. Knockout mice were central to establishing the mechanism. The inability of the peptide amyloid to reduce inflammation in B cell-deficient μMT mice with EAE highlighted the importance of B cells. Flow cytometry and fluorescent microscopy were used to establish that in the peritoneum the relevant target in the B cell population was B-1a lymphocytes, a secretor of IL-10. Additional support for the role of IL-10 secreting B-1a lymphocytes was the failure of IL-10−/− mice with EAE to respond the amyloid therapy. Further support for the role of IL-10-secreting B-1a lymphocytes in the mechanism of action came from classic adoptive transfer experiments. The adoptive transfer of purified B-1a lymphocytes into μMT mice converted the B cell deficient mice from non-responders to responders to the amyloidogenic peptides. An important point was that the transfer of the B-1a lymphocytes alone did not reduce the paralytic signs of EAE. The signs were reduced only after injection of the fibrils, which were shown to activate the transferred population.

Once activated, both the B-1a lymphocytes and the LPMs leave the peritoneum, but their trafficking patterns are less clear. Previous studies established that LPS activation of B-1a lymphocytes resulted in trafficking from the peritoneum to the spleen. Tedder and colleagues have shown activation of a splenic population of regulatory B cells that migrate to draining lymph nodes. Using real-time measurement of the trafficking of adoptively transferred luminescent B-1a lymphocytes and LPMs, revealed that activation with the amyloid fibrils resulted in migration to inguinal lymph nodes. Flow cytometric studies using IL-10 reporter mice supported both the timing and the location of the trafficking.

In the case of an intraperitoneal injection, the activation of the peritoneal cells would be expected to precede any biological activity stimulated by the fibrils in serum, and consequently should contribute to a greater percentage of the response. The proposed mode of action is consistent with the relatively long pharmacokinetics and pharmacodynamics of the amyloidogenic peptides. The fibrils themselves will have an expected half-life measured in minutes. However, the fibrils activate a set of peritoneal cells, which are the therapeutic agents and migrate to the secondary lymph organs, where they secrete immune suppressive IL-10. The cells do not appear to traffic to the sites of inflammation in the spine and the brain in mice with EAE and do not cross the blood-brain barrier, which is consistent with the documented scarcity of B lymphocytes in EAE lesions. The fate of the activated B-1a lymphocytes and the LPMs reflects the pharmacodynamics of the therapy, and not the fate of the amyloidogenic peptides. Similarly in the pharmacokinetics of the response, the one or two day delay in the reduction of the paralytic signs after the injection of the fibrils reflects the time necessary for the activation and migration of the peritoneal cells, combined with the immune suppression of a sufficiently large percentage of the inflammatory T lymphocytes. Reciprocally, cessation of therapy results in a 24 to 72 hour delay in the return of the paralytic signs, which is consistent with the half-life of the immune suppression induced by the IL-10 producing cells, and not the half-life of the fibrils. In many respects the therapeutic effects of the fibrils resemble pharmacokinetics of adoptive cell therapy rather than a classical small molecule therapeutic. The proposed mechanism of the migration of the immune suppressive cells to secondary lymph organs, where they suppress both circulating inflammatory antigen presenting cells and T lymphocytes, is consistent with published studies on B regulatory cells (Baumgarth N, Waffam E E, & Nguyen T T (2015) Annals of the New York Academy of Sciences; Bouaziz J D, Yanaba K, & Tedder T F (2008) Immunological Reviews 224:201-214). The mechanism also argues that this therapeutic approach might be beneficial in a number of systemic inflammatory indications.

The fibrils induce a concomitant inflammatory response, most evidently in the SPMs, with induction of IL-1β, TNFα, and IL-6. Why this response does not dominate the immune suppressive effects can best be explained by the large excess of LPMs and B-1a lymphocytes and their greater propensity to rapidly traffic out of the peritoneum to the secondary lymphoid tissues.

The effective therapy with respiratory tract administration of the fibrils bodes well for translation to human therapy, particularly in light of the predominance of B-1a lymphocytes in the pleural cavity.

The amyloidogenic peptides disclosed herein are the first therapeutic that targets regulatory B cells. The extensive list of indications in which this population of cells limits inflammation is supportive of the potential for the strategy of using the amyloid fibrils in a spectrum of conditions with inflammatory or autoimmune components.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in effectiveness in the EAE animal model of MS.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of activating B-1a lymphocytes to treat an inflammatory or autoimmune condition in a subject in need thereof comprising:
administering to the subject in need thereof a therapeutically effective amount of a formulation comprising a fibril or a hexapeptide fibril-forming peptide having a Rosetta binding energy of −23 kcalmol or less,
thereby activating B-1a lymphocytes and treating the inflammatory or autoimmune condition in the subject in need thereof.

2. The method of claim 1 wherein the inflammatory or autoimmune condition comprises:
multiple sclerosis, rheumatoid arthritis, Huntington's disease, Parkinson's disease, amyotrophic lateral sclerosis, traumatic brain injury, cancer, ischemic reperfusion injury, arthritis asthma, chronic obstructive pulmonary disease (COPD), or inflammatory bowel disease (IBD).

3. The method of claim 1 wherein the administering is to the respiratory tract or the peritoneum.

4. The method of claim 3 wherein the administering to the respiratory tract is to the alveolar region of the lung.

5. The method of claim 1 wherein the fibril or fibril-forming peptide is SEQ ID NO: 1062.

6. The method of claim 1 wherein the fibril-forming peptide is a hexapeptide that oligomerizes with a Rosetta energy at or below −23 kcalmol.

7. The method of claim 1 wherein the fibril or fibril-forming peptide further comprises one or more of SEQ ID NOs: 1-1061.

8. The method of claim 1 wherein the fibril or fibril-forming peptide comprises one or more of SEQ ID NO: 1062; SEQ ID NO: 5; SEQ ID NO: 1048; SEQ ID NO: 1049; SEQ ID NO: 123; SEQ ID NO: 1050; SEQ ID NO: 1051; SEQ ID NO: 791; SEQ ID NO: 1052; SEQ ID NO: 1053; SEQ ID NO: 1054; SEQ ID NO: 1055; SEQ ID NO: 1056; SEQ ID NO: 1057; SEQ ID NO: 220; and SEQ ID NO: 1058.

9. The method of claim 1 wherein the formulation is a conductive formulation.

10. The method of claim 1 wherein the formulation further comprises a mucoactive or mucolytic agent.

11. The method of claim 1 wherein the formulation is a dry powder formulation.

12. The method of claim 1 wherein the formulation further comprises a conductive agent.

13. The method of claim 1 wherein said activating of B-1a lymphocytes occurs in the pleural cavity or peritoneal cavity.

14. A conductive formulation comprising a fibril or fibril-forming peptide and a conductive agent.

15. The conductive formulation of claim 14 wherein the conductive agent is a hypertonic saline solution.

16. The conductive formulation of claim 14 further comprising a mucoactive or mucolytic agent.

17. The conductive formulation of claim 14 wherein the fibril-forming peptide is a hexapeptide that oligomerizes with a Rosetta energy at or below −23 kcal/mol.

18. The conductive formulation of claim 14 wherein the fibril of fibril forming peptide comprises SEQ ID NO: 1062.

19. The conductive formulation of claim 14 wherein the fibril or fibril-forming peptide further comprises one or more peptides selected from SEQ ID NOs: 1-1061.

20. The conductive formulation of claim 14 wherein the fibril or fibril-forming peptide comprises one or more peptides selected from SEQ ID NOs: SEQ ID NO: 1062, SEQ ID NO: 5; SEQ ID NO: 1048; SEQ ID NO: 1049; SEQ ID NO: 123; SEQ ID NO: 1050; SEQ ID NO: 1051; SEQ ID NO: 791; SEQ ID NO: 1052; SEQ ID NO: 1053; SEQ ID NO: 1054; SEQ ID NO: 1055; SEQ ID NO: 1056; SEQ ID NO: 1057; SEQ ID NO: 220; and SEQ ID NO: 1058.
